(19) United States
(12) Patent Application Publication
(10) Pub. No.: US 2019/0008902 A1
(43) Pub. Date: Jan. 10, 2019

(54) METHODS FOR ENHANCED PRODUCTION AND ISOLATION OF CELL-DERIVED VESICLES

(71) Applicant: THE REGENTS OF THE UNIVERSITY OF CALIFORNIA, Oakland, CA (US)

(72) Inventors: Johnathon D. ANDERSON, Davis, CA (US); Jan A. NOLTA, Davis, CA (US); Gerhard BAUER, Davis, CA (US); Brian FURY, Davis, CA (US)

(21) Appl. No.: 16/067,586

(22) PCT Filed: Dec. 30, 2016
(86) PCT No.: PCT/US16/69629

§ 371 Date: Jun. 29, 2018

Related U.S. Application Data
(60) Provisional application No. 62/273,342, filed on Dec. 30, 2015.

(51) Int. Cl. A61K 35/28; C12N 5/0775; C07K 14/71; A61K 9/19; C12M 1/00; A61P 25/00

(52) U.S. Cl. CPC A61K 35/28; C12N 5/0663; C07K 14/71; A61P 25/00; C12M 47/06; C12M 47/12; A61K 9/19

(57) ABSTRACT

This disclosure relates to populations and compositions of purified cell-derived vesicles and uses thereof. One aspect of the disclosure relates to methods for purifying the cell-derived vesicles.

### CROSS REFERENCE TO RELATED APPLICATIONS

This application claims the benefit under 35 U.S.C. § 119(e) of U.S. Provisional Application Ser. No. 62/273,342, filed Dec. 30, 2015, the contents of which are incorporated by reference herein in their entirety including all figures and tables.

### STATEMENT OF GOVERNMENT SUPPORT

This invention was made with United States government support under federal grants NIH Transformative R01GM099688, NSF GRFP 2011116000, NIH T32-GM008799, NSF GROW 201111600, T32-HL086350 awarded by the National Institutes of Health. The United States government has certain rights in the invention.

### SEQUENCE LISTING

The instant application contains a Sequence Listing which has been submitted electronically in ASCII format and is hereby incorporated by reference in its entirety. Said ASCII copy created on Dec. 30, 2016, is named 060933_0642_SL.txt and is 4.05 megabytes in size.

### TECHNICAL FIELD

The invention relates to populations and compositions of purified cell-derived vesicles and uses thereof. One aspect of the disclosure relates to methods for purifying the cell-derived vesicles.

### BACKGROUND

Ischemic tissue related diseases such as peripheral arterial disease (PAD) affect 8-12 million people every year in the U.S. and often there are no satisfactory treatment options for many of these patients. PAD is characterized by a lack of proper blood flow to the lower extremities due to narrowing or blockage of arterial vasculature from atherosclerotic plaques (Milani, R. V. et al. (2007) Vascular Medicine 12(4):351-358). Angioplasty and stent placement are commonly used to treat PAD, however, restenosis and re-occlusion from subsequent blood clot formation and stent overgrowth limit the effectiveness of these treatments in many patients (Katz, G. et al. (2015) Current Atherosclerosis Reports 17(3):485). A potential alternative therapeutic approach is localized induction of angiogenesis to restore blood flow to affected tissues (Banfi, A. et al. (2012) FASEB Journal: Official Publication of the Federation of American Societies for Experimental Biology 26(6):2486-2497). Several studies in animal models of PAD have shown localized induction of angiogenesis via recombinant Vascular Endothelial Growth Factor (VEGF) therapy to be beneficial. However, this straightforward approach has so far failed to show clear benefits in humans in late-stage clinical trials (Yla-Herttuala, S. et al. (2007) Journal of the American College of Cardiology 49(10): 1015-1026).

Mesenchymal stem cells (MSC) facilitate healing of ischemic tissue related diseases, at least in part, through proangiogenic secretory proteins. Recent studies show that MSC derived vesicles function as paracrine effectors of angiogenesis. Exosomes and microvesicles are secreted cellular vesicles of endosomal origin and contain various proteins, lipids, and RNAs from the cytosol of the secreting cells. Upon release into the extracellular space, exosomes and microvesicles function as intercellular messengers, delivering their contents to a recipient target cell.

The identity of the components of the exosome and/or microvesicles, including proteins, responsible for the observed healing effects remains elusive. Identification of the exosome and/or microvesicle components could have a great impact in the treatment of ischemic tissue-related diseases and other diseases. Thus, in order to develop promising vesicle-based therapeutics, there remains a need in the art to identify such components and to modify exosomes to deliver the appropriate factors to a target cell to treat a specific disease.

### SUMMARY

This disclosure relates to purified populations, compositions, and methods of treatment using secreted cell-derived vesicles (e.g., exosomes and/or microvesicles).

One aspect of the disclosure relates to a highly purified population of cell-derived vesicles prepared by culturing stem cells producing the cell-derived vesicles under conditions of hypoxia and low serum conditions, optionally wherein the cell-derived vesicles comprise exosomes and/or microvesicles.

Another aspect of the disclosure relates to a highly purified population of modified cell-derived vesicles, optionally wherein the cell-derived vesicles comprise exosomes and/or microvesicles.

In a further aspect, the disclosure relates to a composition comprising the purified population of cell-derived vesicles according to any one of the embodiments described herein and one or more of a carrier, a preservative or a stabilizing agent.

In one aspect, the disclosure relates to a method for isolating and/or purifying a population of cell-derived vesicles, and in one aspect, exosomes, the method comprising, or consisting essentially of, or yet further consisting of: (a) isolating the cell-derived vesicles from conditioned media containing the cell-derived vesicles by an appropriate method, e.g., by applying a tangential flow filtration to conditioned media produced by a population of isolated stem cells to isolate a cell-derived vesicle containing fraction; and (b) concentrating the cell-derived vesicle containing fraction to provide a purified population of cell-derived vesicles. Any appropriate method can be used to concentrate the cell-derived vesicles, e.g. exosomes. Non-limiting examples of such include centrifugation, ultrafiltration, filtration, differential centrifugation and column filtration with a 100 kDA to 300 kDa pore size, or either a 100 kDA to 300 kDa pore size. Further sub-populations can be isolated using antibodies or other agents that are specific for a specific marker expressed by the desired exosome population.

In another aspect, prior to isolation and/or purification of the cell-vesicles, the stem cells producing the vesicles are grown or cultured by any method known in the art, e.g. by a method comprising the use of a hollow fiber bioreactor prior to the isolation and/or purification of the cell-derived vesicles from the conditioned media. In one aspect, the cell-derived vesicles are exosomes. In one aspect, the stem cells (that produce the conditioned media containing the cell-derived vesicles and/or exosomes) are cultured under conditions of low serum and hypoxia or low oxygen conditions.

In some embodiments, the cell-derived vesicles of the population further comprise at least one exogenous nucleic acid and/or at least one exogenous protein, i.e. a nucleic acid or protein that is not present in a naturally occurring cell-vesicle. Alternatively, the cell-derived vesicles can further comprise an endogenous nucleic acid and/or endogenous protein that is naturally present in the cell-derived vesicle but whose expression is to be enhanced or inhibited. Non-limiting examples of nucleic acids include one or more or all of DNA and RNA, for example mRNA, RNAi, siRNA, pcRNA. In some embodiments, the exogenous or endogenous nucleic acid encodes one or more of a micro RNA (miRNA), for example, miR-181, miR-210, miR-214, miR-424, miR-150, miR-126, miR-132, miR-296, or let-7. In some embodiments, the exogenous or endogenous protein is one or more of platelet derived growth factor receptor (PDGFR), Collagen, Type 1, Alpha 2 (COL1A2), Collagen, Type VI, Alpha 3 (COL6A3), EGF-like repeats- and discoidin i-like domains-containing protein 3 (EDIL3), epidermal growth factor receptor (EGFR), fibroblast growth factor receptor (FGFR), fibronectin (FN1), Milk fat globule-EGF factor 8 (MFGE8), lectin, galactoside-binding, soluble, 3 binding protein (LGALS3BP), nuclear factor-kappaB (NFκB), transferrin (TF), vascular endothelial growth factor (VEGF), VEGF isoform 165A, or vascular endothelial growth factor receptor (VEGFR). In other embodiments, the population of cell-derived vesicles do not express or comprise VEGF, VEGFR or both. In some embodiments, the cell-derived vesicles of the present disclosure are modified to comprise one or more of an exogenous or endogenous protein, nucleic acid, metabolite, lipid, and/or membrane component, that can be detected in the exosomes and/or microvesicles of the present disclosure.

In some embodiments, the cell-derived vesicles of the population further comprise at least one exogenous nucleic acid and/or at least one exogenous protein, i.e. a nucleic acid or protein that is not present in a naturally occurring cell-vesicle. Alternatively, the cell-derived vesicles can further comprise an exogenous nucleic acid and/or exogenous protein that is naturally present in the cell-derived vesicle but whose expression is to be enhanced or inhibited. Non-limiting examples of nucleic acids include one or more or all of DNA and RNA, for example mRNA, RNAi, siRNA, pcRNA. In some embodiments, the exogenous nucleic acid encodes one or more of a micro RNA (miRNA), for example, miR-181, miR-210, miR-214, miR-424, miR-150, miR-126, miR-132, miR-296, or let-7. In some embodiments, the exogenous protein is one or more of platelet derived growth factor receptor (PDGFR), Collagen, Type 1, Alpha 2 (COL1A2), Collagen, Type VI, Alpha 3 (COL6A3), EGF-like repeats- and discoidin i-like domains-containing protein 3 (EDIL3), epidermal growth factor receptor (EGFR), fibroblast growth factor receptor (FGFR), fibronectin (FN1), Milk fat globule-EGF factor 8 (MFGE8), lectin, galactoside-binding, soluble, 3 binding protein (LGALS3BP), nuclear factor-kappaB (NFκB), transferrin (TF), vascular endothelial growth factor (VEGF), VEGF isoform 165A, or vascular endothelial growth factor receptor (VEGFR). In other embodiments, the population of cell-derived vesicles do not express or comprise exogenous VEGF, VEGFR or both. In some embodiments, the cell-derived vesicles of the present disclosure are modified to comprise one or more of an exogenous protein, nucleic acid, metabolite, lipid, and/or membrane component, that can be detected in the exosomes and/or microvesicles of the present disclosure, (and listed in the molecular composition of exosomes section below).

A non-limiting example of a method and composition to provide a purified and/or isolated population of cell-derived vesicles comprising at least one exogenous nucleic acid is by transforming an isolated host cell, such as a stem cell with a vector comprising the coding polynucleotide. SEQ ID NO: 18 is an example of such a vector. Thus, in another aspect, provided herein is a lentiviral vector comprising the necessary regulatory elements. As is apparent to the skilled artisan, the marker sequence (nucleotides 5894 to 7321 of SEQ ID NO: 18) can be omitted as well as the enhancer element (nucleotides 7345 to 7941 of SEQ ID NO: 18) or be substituted with alternative markers or enhancers. In addition, nucleotides 5208 to 5363 correspond to the miR-132 element but other elements, as described herein or as known in the art, can be substituted therein. Alternative promoters (the PGK promoter provided as nucleotides 5364 to 5874) can be substituted as well. Alternative vectors are described in U.S. Patent Publication No. 2016/0046685 and WO 2014/035433, each incorporated by reference herein. One disclosed vector of WO 2014/035433 contains a gene encoding for the 165A isoform of VEGF and includes an MNDU3 promoter and an optional enhancer element.

Isolated host cells, such as stem cells, comprising such vectors are further provided as well as populations of such cells alone or in combination with the isolated or purified cell-derived vesicles as described herein. These compositions can be further combined with a carrier, preservative or stabilizer.

Also provided are methods for preparing the cell-derived vesicles by culturing the host cells to grow the cells, also as provided herein. As noted in more detail herein, in one aspect, mesenchymal stem cells were transfected with a plasmid expression vector overexpressing miR-132 and tdTomato marker (SEQ ID NO: 18). Microvesicles were harvested from media that had been conditioned for 48 hours using ultracentrifugation.

In some embodiments, the population of cell-derived vesicles or isolated host cells is substantially homogeneous. In other embodiments, the population of cell-derived vesicles or isolated host cells is heterogeneous.

In some embodiments, the concentration of cell-derived vesicles in or isolated from the population comprises between about 0.5 micrograms to about 200 micrograms of cell-derived vesicle protein collected per approximately 10⁶ cells. In some embodiments, the concentration of cell-derived vesicles in or isolated from the population comprises between about 200 micrograms to about 5000 micrograms of cell-derived vesicle protein collected per approximately 10⁶ cells. In other embodiments, the concentration of cell-derived vesicles in or isolated from the population comprises less than about 5000, or alternatively less than about 1000, or alternatively less than about 500, or alternatively less than about 200, or alternatively less than about 150, or alternatively less than about 125, or alternatively less than about 100, or alternatively less than about 75, or alternatively less than about 50, or alternatively less than about 30 micrograms, or alternatively less than about 25 micrograms, of cell-derived vesicle protein collected per approximately 10⁶ cells. In yet other embodiments, the concentration of cell-derived vesicle protein in or isolated from the population is less than about 20 micrograms per 10⁶ cells.

In some embodiments, the average diameter of the cell-derived vesicles in or isolated from the population is between about 0.1 nm and about 1000 nm, or alternatively between about 1.0 nm and about 1000 nm, or alternatively between about 1.5 nm and about 1000 nm. In other embodiments, the average diameter is between about 2 nm and about 800 nm, or alternatively about 2 nm to about 700 nm, or alternatively from about 2 nm to about 600 nm, or alternatively from about 2 nm to about 500 nm, or alternatively from about 2 nm to about 400 nm, or alternatively from about 2 nm to about 300 nm. In other embodiments, the average diameter is between about 10 nm and about 1000 nm, or alternatively 100 nm to about 1000 nm, or alternatively from about 300 nm to about 1000 nm, or alternatively from about 500 nm to about 1000 nm, or alternatively from about 750 nm to about 1000 nm, or alternatively from about 800 nm to about 1000 nm. In other embodiments, the average diameter of the cell-derived vesicles in or isolated from the population is less than about 100 nm. In further embodiments, the average diameter of the cell-derived vesicles in or isolated from the population is less than about 50 nm. In still further embodiments, the average diameter of the cell-derived vesicles in the population is less than about 40 nm.

In some embodiments, the purified population of cell-derived vesicles described herein have been purified from by a methods known in the art, e.g. by a method comprising tangential flow filtration or other filtration method. Prior to isolation, the cells producing the cell-derived vesicles can be cultured by any appropriate method known in the art, e.g., in a hollow-fiber bioreactor.

In some embodiments, the population of cell-derived vesicles, e.g., exosomes is combined with a carrier, for example, a pharmaceutically acceptable carrier, that in one aspect, provides the composition with enhanced stability over an extended period of time. The compositions can be further combined with other therapeutic agents, e.g. an angiogenesis promoter, a phytochemical agent, a chemotherapeutic agent, and/or a Stat3 inhibitor, that in one aspect, are encapsulated by the exosome. Non-limiting examples of angiogenesis promoters include, angiotensin, prostaglandin E₁ (PGE₁), modified PGE₁ (see U.S. Pat. No. 6,288,113, incorporated by reference herein) and angiopoietin-1. Methods to encapsulate agents within exosomes are known in the art and described for example in U.S. Patent Publication No. 2014/0093557, published Apr. 3, 2014, and incorporated by reference herein. In some embodiments, the compositions are formulated for therapeutic application and/or enhanced stability such as by drying, freeze drying, snap-freezing, or lyophilization.

In some embodiments, the compositions described herein further comprise an isolated stem cell, for example, one or more of an adult stem cell, an embryonic stem cell, an induced pluripotent stem cell, an embryonic-like stem cell, a mesenchymal stem cell, or a neural stem cell. In one aspect, the isolated stem cell further is modified, for example by the introduction of a vector and/or gene for therapeutic use. A non-limiting example of such is a stem cell modified to express a pro-angiogenic factor, e.g., VEGF or an equivalent thereof as described in U.S. Patent Publication No. 2016/0046685 and WO 2014/035433, each incorporated by reference herein. The compositions can be further combined with other therapeutic agents, e.g. an angiogenesis promoter, a phytochemical agent, a chemotherapeutic agent, and/or a Stat3 inhibitor.

In a further aspect, the disclosure relates to a method for promoting angiogenesis in a subject in need thereof comprising administering to the subject an effective amount of a purified population and/or a composition according to any one of the embodiments described herein. The methods can further comprise administration of an effective amount of other agents, e.g. agents that facilitate or promote angiogenesis, e.g., angiotensin, prostaglandin E₁ (PGE₁), modified PGE₁ (see U.S. Pat. No. 6,288,113, incorporated by reference herein) and angiopoietin-1. The administration can be concurrent or sequential as determined by the treating physician. The subject can be an animal, e.g., a mammal such as a human patient in need of such treatment, that in one aspect, has been pre-selected for the therapy by a treating physician or other health care professional.

In a further aspect, the disclosure relates to a method for treating peripheral arterial disease or stroke comprising administering to a subject an effective amount of a purified population and/or a composition according to any one of the embodiments described herein. The methods can further comprise administration of an effective amount of other agents, e.g., agents that facilitate or promote angiogenesis, e.g., angiotensin, prostaglandin E₁ (PGE₁), modified PGE₁ (see U.S. Pat. No. 6,288,113, incorporated by reference herein) and angiopoietin-1. The administration can be concurrent or sequential as determined by the treating physician. The subject can be an animal, e.g., a mammal such as a human patient in need of such treatment, that in one aspect, has been pre-selected for the therapy by a treating physician or other health care professional.

In yet a further aspect, the disclosure relates to a method for treating a dermal wound in a subject comprising administering to the subject an effective amount of a purified population and/or a composition according to any one of the embodiments described herein. The methods can further comprise administration of an effective amount of other agents, e.g., agents that facilitate or promote angiogenesis, e.g., angiotensin, prostaglandin E₁ (PGE₁), modified PGE₁ (see U.S. Pat. No. 6,288,113, incorporated by reference herein) and angiopoietin-1. The administration can be concurrent or sequential as determined by the treating physician. The subject can be an animal, e.g., a mammal such as a human patient in need of such treatment, that in one aspect, has been pre-selected for the therapy by a treating physician or other health care professional.

In some embodiments, the subject is administered at least one dose of between approximately 0.1 mg and 200 mg of cell-derived vesicle protein. In other embodiments, the subject is administered at least one dose of approximately 50 mg of cell-derived vesicle protein.

In some embodiments, the purified population and/or the composition according to any one of the embodiments as described herein is administered prior to or after administration of an isolated stem cell that may optionally be modified. In other embodiments, the purified population and/or the composition according to any one of the embodiments as described herein is administered simultaneously with an isolated stem cell. In one aspect, the stem cell has been transduced with VEGF or a VEGF isoform, as described above.

In some embodiments, the purified population and/or the composition according to any one of the embodiments as described herein, is administered by intravenous injection, direct injection, intramuscular injection, intracranial injection, or topically.

In some embodiments, the subject is a mammal, optionally a human patient. In a further aspect, the patient has been selected for the therapy by diagnostic criteria as known to those of skill in the art.

In some embodiments, according to the methods described herein, e.g., a method for purifying a population of cell-derived vesicles, comprising: (a) applying a tangential flow filtration to conditioned media produced by a population of isolated stem cells to isolate a cell-derived vesicles containing fraction; and (b) concentrating the cell-derived vesicle containing fraction to provide a purified population of cell-derived vesicles. after step (a) cell debris and other contaminates are removed from the cell-derived vesicle containing fraction prior to step (b). In some embodiments, according to the methods described herein, the population of stem cells are cultured under hypoxic and low serum conditions for up to about 72 hours prior to performing step (a). In some embodiments, according to the methods described herein, step (a) is performed using an approximately 200 nanometer filter.

In some embodiments, according to the methods described herein, the isolated stem cells that produce the cell-derived vesicles are one or more of adult stem cells, embryonic stem cells, embryonic-like stem cells, neural stem cells, or induced pluripotent stem cells. In some embodiments, the stem cells are mesenchymal stem cells that in one aspect, are cultured under hypoxic and low serum conditions.

In some embodiments, according to the methods described herein, the hypoxic conditions are between approximately 1% to about 15% CO₂, for example about 5% CO₂, and between about 0.05% to about 20% oxygen tension. In some embodiments, the low serum conditions are serum free conditions.

In some embodiments, according to the methods described herein, the tangential flow filtration unit used for isolation and/or purification of the cell-derived vesicles is between about 50 kilodalton and about 400 kilodalton nominal molecular weight limit filtration unit, for example, about a 100 kilodalton nominal molecular weight limit filtration unit or about a 300 kilodalton nominal molecular weight limit filtration unit.

In some embodiments, the methods described herein further comprise formulating the purified population of cell-derived vesicles by mixing the population with a carrier and/or another therapeutic agent either by admixing the components or by encapsulation of the therapeutic agent using methods known in the art.

In some embodiments, the methods described herein further comprise freezing or freeze drying the purified population of cell-derived vesicles and/or compositions.

Also provided herein are populations of cell-derived vesicles obtainable from the methods according to any one of the embodiments as described herein.

Further provided herein are lyophilized or frozen populations of cell-derived vesicles of the purified population or the composition according to any one of the embodiments as described herein.

Still further provided herein are kits comprising populations of cell-derived vesicles of any one of the embodiments as described herein and instructions for use.

In a further aspect, the disclosure relates to a method for large-scale purification of a population of cell-derived vesicles, comprising applying a tangential flow filtration to conditioned media produced by a population of isolated stem cells cultured in a bioreactor to isolate a cell-derived vesicles containing fraction; and concentrating the cell-derived vesicle containing fraction to provide a purified population of cell-derived vesicles.

### BRIEF DESCRIPTION OF DRAWINGS

FIGS. 1A to 1C show experimental design workflow and ratio distribution of MSC proteomics. (A) Schematic representation of proteomics workflow. MSCs were isolated from human bone marrow and expanded to passage 6 using expansion (EX) conditions. Cells were then washed 3 times with PBS and switched to either expansion (EX), intermediate (IC) or PAD-like (PAD) conditions for 40 hours. Cells or exosomes were then lysed, trypsinized and ran on high-resolution isoelectric focusing (HiRIEF) strips which were divided into 72 individual fractions and ran on liquid chromatography tandem mass spectrometry (LC-MS/MS). Identified proteins were analyzed using 3 different types of analysis software: gene ontology, canonical signaling pathways and network analysis of the angiome interactome. ClueGO gene ontology analysis was used to characterize enrichment for proteins based on their functionalities. Panther and IPA pathway analysis was used to characterize enrichment for proteins of specific canonical signaling pathways. CytoScape network analysis of the angiome interactome was used to visualize the physical interactions of known angiogenesis-mediating proteins (angiome) with proteins for which there is experimental evidence of physical interaction. (B) Plot of PAD/EX ratios (Log 2, fold change) versus area (Log 10, abundance) of MSC proteins; dots represent significantly differentially expressed proteins (FDR1%), all non-significantly differentially expressed proteins. (C) PAD/EX ratios (Log 2, fold change) versus P-value; differentially expressed proteins with mean fold changes <+/−0.5 Log 2, and >+/−0.5 Log 2 mean fold change with p-value <0.01 and blue dots with a p-value of >0.01.

FIGS. 2A and 2B show analysis of HiRIEF LC-MS/MS proteomics data from IC and PAD conditions compared to control condition EX. (A) Heatmap of MSC cluster analysis of differentially regulated proteins in IC and PAD conditions as compared to EX. (B) Panther pathway analysis of proteins upregulated in MSCs under PAD-like conditions show abundance of canonical angiogenesis related pathway proteins: EGF, FGF and PDGF (red asterisk indicate angiogenesis associated pathways). Analysis of 3 different donors for each condition. For differential expression T-tests with multiple testing correction with an FDR of 1% was used. Circles are color coded according to their associated functionality. Number of circles and larger diameter of circles indicate greater over representation.

FIGS. 3A to 3D show mesenchymal stem cells increase secretion of exosomes upon exposure to PAD-like conditions. (A) Quantification of total protein content of vesicles derived from MSC under EX, IC and PAD culture conditions using DC assay. (B) Scanning electron micrograph of MSCs cultured in EX culture conditions indicating microvesicle release (arrows) from the cell surface (scale bar 5 um, 5kX). (C) Scanning electron micrograph of MSCs cultured under PAD conditions (scale bar 2 um, 10kX) indicating exosome adhesion to cell surface (arrows). (D) Transmission electron micrograph of MSC derived exosomes with 2% uranyl acetate negative staining (scale bar 200 nm, 25kX).

FIG. 4 shows analysis of HiRIEF LC-MS/MS proteomics data of MSC exosomes comparing PAD to IC conditions. Panther pathway analysis of PAD exosomes shows abundance of angiogenesis related pathway proteins: EGFR, FGF and PDGF pathway associated proteins (red asterisk indicate angiogenesis associated pathways). Analysis of 3 different donors for each condition. For differential expression T-tests with multiple testing correction with an FDR of 1% was used.

FIGS. 5A to 5F show MSC exosome-induced in vitro tubule formation of HUVECs. (A) Basal media (Neg), (B) 5 μg/ml, (C) 10 ug/ml, (D) 20 ug/ml of MSC exosomes in basal media, (E) EndoGRO media positive control (Pos). Stained with Calcein AM and imaged at 14 hours post stimulation with 4× objective. (F) Quantification of total segment length of tubule formation analyzed using ImageJ's Angiogenesis plugin. EndoGRO positive control media contains 2% FBS, EGF 5 ng/ml and heparin sulfate 0.75 U/ml. (*) Indicates a p-value <0.05 using ANOVA, LSD post hoc analysis (n=12).

FIGS. 6A to 6G show NFkB inhibition abrogates MSC exosome-mediated tubule formation in HUVECs in vitro. (A) basal media, (B) basal media+NFkB inhibitor, (C) 10 ug/ml, (D) 10 ug/ml+NFkB inhibitor, (E) EndoGRO media, (F) EndoGRO media+NFkB inhibitor. HUVECs stained with Calcein AM and imaged 14 hours post stimulation with a 4× objective. (G) Quantification of total segment length of tubule formation using ImageJ's Angiogenesis plugin. EndoGRO media contains 2% FBS, EGF 5 ng/ml and heparin sulfate 0.75 U/ml. (*) Indicates a p-value <0.01 using ANOVA, LSD post hoc analysis (n=6).

FIG. 7 shows detection of MSC membrane associated proteins. Venn diagram showing overlap of detected membrane associated proteins between consensus cellular MSC HiRIEF LC-MS/MS data (detected in all 9 samples) and the consensus Mindaye et al. MSC proteome dataset (detected in all 4 samples) and the Uniprot human proteome database.

FIGS. 8A and 8B show representative concordance and variation between MSC donors. (A) Heatmap of cellular global proteome expression differentials between IC/EX and PAD/EX across all 3 donors reveals some donor to donor variation as well as intra-condition and intra-donor concordance. (B) Comparison of PAD/EX donor ratios from all 3 donors reveals some donor to donor variation as well as intra-condition and intra-donor concordance. Dots represent PAD/EX protein expression ratios of donor 3 vs donor land PAD/EX protein expression ratios of donor 2 vs donor 1. Line represents regression analysis of PAD/EX protein expression ratios of donor 3 vs donor land regression analysis of PAD/EX protein expression ratios of donor 2 vs donor 1.

FIGS. 9A and 9B show upregulation of glycolysis pathway proteins in PAD/EX. Ingenuity Pathway Analysis of differentially expressed cellular proteins (FDR-1%) revealed increased expression of key regulators of glycolysis in the PAD condition as compared to the EX condition. The first half of the pathway is illustrated in (A) and the second half of the pathway is illustrated in (B). Analysis of 3 different donors per condition. For differential expression T-tests with multiple testing correction with an FDR of 1% was used.

FIG. 10 shows upregulation of cholesterol biosynthesis pathway proteins in PAD/EX. Ingenuity Pathway Analysis of differentially expressed cellular proteins (FDR-1%) revealed upregulation of proteins associated with the cholesterol biosynthesis pathway in the PAD condition as compared to the EX condition. Dark gray boxes indicate increased expression, light gray boxes indicate lack of detection. Analysis of 3 different donors per condition. For differential expression T-tests with multiple testing correction with an FDR of 1% was used.

FIGS. 11A and 11B show upregulation of exosome biogenesis proteins in PAD/EX. (A) Relative expression of known exosome biogenesis proteins demonstrated a trend towards increased expression in PAD/EX. (B) Vesicle associated protein family members demonstrated a trend towards increased expression in PAD/EX.

FIG. 12A shows size distribution analysis of MSC exosomes. FIG. 12 B shows nanosight tracking analysis showing the size distribution of MSC exosome and relative intensity.

FIGS. 13A to 13C show exosomal delivery of functional exogenous mRNA to endothelial cells. (A) tdTomato mRNA was packaged into exosomes derived from MSC-PAD transduced with a lentiviral vector expression vector and functionally delivered to endothelial cells. Imaging was performed at (B) 8 hours and (C) 72 hours after exosome exposure.

FIG. 14 shows PCR detection of plasmid expression vector in MSC microvesicles.

FIG. 15 shows microvesicle delivery of functional plasmid expression vector to endothelial cells. A tdTomato plasmid expression vector was packaged into microvesicles derived from transfected MSCs and functionally delivered to primary endothelial cells. Cells were imaged 48 hours post-microvesicle exposure.

FIG. 16 shows a schematic representation of the different types of membrane vesicles released by eukaryotic cells, either by direct budding from the plasma membrane (e.g., microvesicles) or by fusion of internal multivesicular endosomes (MVE) with the plasma membrane (e.g., exosomes).

FIG. 17 shows quantitative PCR (qPCR) detection of miR-132 in microvesicles isolated from MSCs modified with a miR-132 lentiviral vector.

FIGS. 18A to 18C show composition of MSC-Stroke exosomes. (A) Bioanalyzer analysis of MSC-Stroke exosomes demonstrated enrichment for small RNAs. (B) qPCR analysis determined presence of angiogenic miRNAs demonstrating their presence at various concentrations, normalized to U6. (C) Log scale relative abundance of RNA and proteins (ng) in MSC-Stroke exosomes, T-test *=p<0.05.

FIG. 19 shows that MSC-Stroke exosomes are packaged with lipid membrane components with signaling functions. Hydrophilic interaction chromatography mass spectrometry analysis (FDR 1%) demonstrates that MSC-Stroke exosomes are packaged lipid bilayer membrane components and their derivatives with important signaling functions include sphingomyelin (SM), phosphatidylcholines (PC), phosphatidyethanolamine (PE) and fatty acids (FA), many of which are also important for the biogenesis of exosomes.

FIG. 20 shows exosome yield based on total exosomal protein content of standard cell culture flasks, 50× T175's vs GMP grade bioreactor. This data demonstrates that GMP-grade manufacturing using a hollow fiber reactor system generates much higher yields of exosomes as compared to standard tissue culture flasks.

FIG. 21 shows transmission electron microscopy with uranyl acetate negative staining. This figure shows that GMP-grade manufacturing using a hollow fiber reactor system generates exosomes of canonical morphology and diameter.

FIG. 22 shows a list of metabolites detected within exosomes and/or microvesicles of the present disclosure.

FIGS. 23A and 23B show a list of lipids and/or membrane components detected within exosomes and/or microvesicles of the present disclosure. (A) comprises the first two thirds of the list and (B) comprises the final third of the list.

FIG. 24 shows a list of proteins associated with angiogenesis that were detected within exosomes and/or microvesicles of the present disclosure.

FIG. 25 shows a list of proteins associated with immune modulation detected within exosomes and/or microvesicles of the present disclosure.

FIG. 26 shows a list of therapeutic proteins detected within exosomes and/or microvesicles of the present disclosure.

FIG. 27 shows a list of canonical exosome-associated proteins detected within exosomes and/or microvesicles of the present disclosure.

### DESCRIPTION OF EMBODIMENTS

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated, that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a cell” includes a plurality of cells.

### Definitions

The following definitions assist in defining the meets and bounds of the inventions as described herein. Unless specifically noted, the embodiments describing “cell-derived vesicles” shall include “exosomes,” “microvesicles” alone or in combination.

The term “about” when used before a numerical designation, e.g., temperature, time, amount, concentration, and such other, including a range, indicates approximations which may vary by (+) or (−) 10%, 5% or 1%.

The terms “administering” or “administration” in reference to delivering cell-derived vesicles to a subject include any route of introducing or delivering to a subject the cell-derived vesicles to perform the intended function. Administration can be carried out by any suitable route, including orally, intranasally, parenterally (intravenously, intramuscularly, intraperitoneally, or subcutaneously), intracranially, or topically. Additional routes of administration include intraorbital, infusion, intraarterial, intracapsular, intracardiac, intradermal, intrapulmonary, intraspinal, intrasternal, intrathecal, intrauterine, intravenous, subarachnoid, subcapsular, subcutaneous, transmucosal, or transtracheal. Administration includes self-administration and the administration by another.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, the term “modified,” relative to cell-derived vesicles, refers to cell-derived vesicles (e.g., exosomes and/or microvesicles) that have been altered such that they differ from a naturally occurring cell-derived vesicles. Non-limiting examples of a modified cell-derived vesicle include an exosome and/or microvesicle that contains a nucleic acid or protein of a type or in an amount different than that found in a naturally occurring exosome and/or microvesicle.

The terms “patient,” “subject,” or “mammalian subject” are used interchangeably herein and include any mammal in need of the treatment or prophylactic methods described herein (e.g., methods for the treatment or prophylaxis of PAD). Such mammals include, particularly humans (e.g., fetal humans, human infants, human teens, human adults, etc.). Other mammals in need of such treatment or prophylaxis can include non-human mammals such as dogs, cats, or other domesticated animals, horses, livestock, laboratory animals (e.g., lagomorphs, non-human primates, etc.), and the like. The subject may be male or female. In certain embodiments the subject is at risk, but asymptomatic for PAD. McDermott et al. (2008) Circulation 117(19) 2484-2491. In certain embodiments, the subject expresses symptoms of PAD, e.g., intermittent claudication (muscle pain, cramping of arms or legs), leg numbness or weakness, change of color of legs, weak or no pulse, and erectile dysfunction in men.

The term “purified population,” relative to cell-derived vesicles, as used herein refers to plurality of cell-derived vesicles that have undergone one or more processes of selection for the enrichment or isolation of the desired exosome population relative to some or all of some other component with which cell-derived vesicles are normally found in culture media. Alternatively, “purified” can refer to the removal or reduction of residual undesired components found in the conditioned media (e.g., cell debris, soluble proteins, etc.). A “highly purified population” as used herein, refers to a population of cell-derived vesicles in which at least 65%, at least 70%, at least 75%, at least 80%, at least 85%, at least 90%, at least 95%, at least 98%, at least 99% or 100% of cell debris and soluble proteins (e.g., proteins derived from fetal bovine serum and the like) in the conditioned media along with the cell-derived vesicles are removed.

The terms “treatment,” “treat,” “treating,” etc. as used herein, include but are not limited to, alleviating a symptom of a disease or condition (e.g., peripheral arterial disease (“PAD”) or a condition associated with PAD) and/or reducing, suppressing, inhibiting, lessening, ameliorating or affecting the progression, severity, and/or scope of the disease or condition. Additional treatments include promoting angiogenesis, treating stroke, treating wounds, treating ischemia, acute and chronic limb ischemia, Buerger's disease, and critical limb ischemia in diabetes. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented.

The term “stem cell” refers to a cell that is in an undifferentiated or partially differentiated state and has the capacity to self-renew and to generate differentiated progeny. Self-renewal is defined as the capability of a stem cell to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells (MSCs) and neural stem cells (NSCs). In some embodiments, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

A “mesenchymal stem cell,” or MSC, is a multipotent stem cell that can differentiate into a variety of cell types. Cell types that MSCs have been shown to differentiate into in vitro or in vivo include osteoblasts, chondrocytes, myocytes, and adipocytes. Mesenchyme is embryonic connective tissue that is derived from the mesoderm and that differentiates into hematopoietic and connective tissue, whereas MSCs do not differentiate into hematopoietic cells. Stromal cells are connective tissue cells that form the supportive structure in which the functional cells of the tissue reside. Methods to isolate such cells, propagate and differentiate such cells are known in the technical and patent literature, e.g., U.S. Patent Publication Nos. 2007/0224171, 2007/0054399, 2009/0010895, which are incorporated by reference in their entirety. In one embodiment, the MSCs are plastic-adherent when maintained in standard culture conditions. In one embodiment, the MSC has the phenotype CD34⁻/CD45⁻/CD105⁺/CD90⁺/CD73⁺. In another embodiment, the MSC has the phenotype CD45⁻/CD34⁻/CD14⁻ or CD11b⁻/CD79a⁻ or CD19⁻/HLA-DR⁻ or HLA-DR^{low}/CD105⁺/CD90⁺/CD73⁺.

The term “induced pluripotent stem cells” as used herein is given its ordinary meaning and also refers to differentiated mammalian somatic cells (e.g., adult somatic cells, such as skin) that have been reprogrammed to exhibit at least one characteristic of pluripotency. See, for example, Takahashi et al. (2007) Cell 131(5):861-872, Kim et al. (2011) Proc. Natl. Acad. Sci. 108(19): 7838-7843, Sell, S. Stem Cells Handbook. New York: Springer, 2013. Print.

The term “exogenous” in reference to a nucleic acid or protein refers to a polynucleotide or polypeptide sequence that has been artificially introduced into a cell, cell-derived vesicles, exosomes, microvesicle, or combination thereof. There may be an endogenous nucleic acid or protein having the same or substantially similar sequence as that of the polynucleotide or polypeptide encoding the exogenous nucleic acid or protein in the cell-derived vesicles or they may be a non-naturally occurring nucleic acid or protein to the a cell, cell-derived vesicles, exosomes, microvesicle, or combination thereof. For example, a mesenchymal stem cell can be genetically modified to overexpress a PDGFR-encoding polynucleotide. It is contemplated that a purified population of cell-derived vesicles isolated from the culture media collected from MSCs genetically modified to overexpress a gene or protein e.g., PDGFR would contain higher levels of PDGFR as compared to cell-derived vesicles isolated from MSCs that have not been modified to overexpress a PDGFR-encoding polynucleotide.

As used herein, the term “microRNAs” or “miRNAs” refers to post-transcriptional regulators that typically bind to complementary sequences in the three prime untranslated regions (3′ UTRs) of target messenger RNA transcripts (mRNAs), usually resulting in gene silencing. Typically, miRNAs are short, non-coding ribonucleic acid (RNA) molecules, for example, 21 or 22 nucleotides long. The terms “microRNA” and “miRNA” are used interchangeably.

As used herein, the terms “overexpress,” “overexpression,” and the like are intended to encompass increasing the expression of a nucleic acid or a protein to a level greater than the exosome naturally contains. It is intended that the term encompass overexpression of endogenous, as well as heterologous nucleic acids and proteins.

As used herein, the term “homogeneous” in reference to a population of cell-derived vesicles refers to population of cell-derived vesicles that have a similar amount of an exogenous nucleic acid, a similar amount of an exogenous protein, are of a similar size, or combinations thereof. A homogenous population is one wherein about 65%, about 70%, about 75%, about 80%, about 85%, about 90%, about 95%, about 98%, or 100% of the cell-derived vesicles share at least one characteristic. For example, in some embodiments about 90% of the cell-derived vesicles in the homogenous purified population overexpress miR-132. For example, in some embodiments about 90% of the cell-derived vesicles in the homogenous purified population overexpress miR-132 wherein the miR-132 is expressed at an amount that is at least 2 times greater than that typically found in cell-derived vesicles. Another example of a homogenous population is one wherein about 90% of the exosomes are less than 50 nm in diameter.

As used herein, the term “heterogeneous” in reference to a population of cell-derived vesicles refers to population of cell-derived vesicles that have differing amounts of an exogenous nucleic acid, differing amounts of an exogenous protein, are of a different size, or combinations thereof.

The term “substantially” refers to the complete or nearly complete extent or degree of a characteristic and in some aspects, defines the purity of the isolated or purified population of exosomes or microvesicle. For example, a substantially homogenous cell-derived vesicle population may be a cell-derived vesicle population that contains more than 60%, more than 70%, more than 80%, more than 90%, more than 95%, more than 98%, or 100% cell-derived vesicles that comprise at least one exogenous nucleic acid, protein, or both.

As used herein, the term “tangential-flow filtration” (TFF) refers to a process in which the fluid mixture containing the cell-derived vesicles to be separated by filtration is recirculated at high velocities tangential to the plane of the membrane to increase the mass-transfer coefficient for back diffusion. In such filtrations a pressure differential is applied along the length of the membrane to cause the fluid and filterable solutes to flow through the filter. This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream. Tangential flow may contain cassette filters or cartridge (also called hollow fiber) filters that the membrane forms a set of parallel hollow fibers. The feed stream passes through the lumen of the fibers and the permeate is collected from outside the fibers. Cartridges are characterized in terms of fiber length, lumen diameter and number of fibers, as well as filter pore size.

As used herein, the term “pharmaceutically acceptable carrier” refers to any of the standard pharmaceutical carriers such as sterile solutions, tablets, coated tablets, and capsules. Typically such carriers contain excipients such as starch, milk, sugar, certain types of clay, gelatin, stearic acids or salts thereof, magnesium or calcium stearate, talc, vegetable fats or oils, gums, glycols, or other known excipients. Such carriers may also include flavor and color additives or other ingredients. Examples of pharmaceutically acceptable carriers include, but are not limited to, the following: water, saline, buffers, inert, nontoxic solids (e.g., mannitol, talc). Compositions comprising such carriers are formulated by well-known conventional methods. Depending on the intended mode of administration and the intended use, the compositions may be in the form of solid, semi-solid, or liquid dosage forms, such, for example, as powders, granules, crystals, liquids, suspensions, liposomes, pastes, creams, salves, etc., and may be in unit-dosage forms suitable for administration of relatively precise dosages.

An “effective amount” intends an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks.

As used herein, the term “peripheral arterial disease” or “PAD” refers is a subset of peripheral vascular disease. Peripheral arterial disease or peripheral artery disease can occur in arteries other than those supplying blood to the heart, but most often occurs in the legs and feet. The disease is characterized by segmental lesions causing stenosis or occlusion, usually in large and medium-sized arteries. Atherosclerosis is the leading cause of PAD, which results in atherosclerotic plaques with calcium deposition, thinning of the media, patchy destruction of muscle and elastic fibers, fragmentation of the internal elastic lamina, and thrombi composed of platelets and fibrin. Common sites for PAD are the femoral and popliteal arteries, (80 to 90% of patients), the abdominal aorta and iliac arteries (30% of patients) and the distal vessels, including the tibial artery and peroneal artery (40-50% of patients). The incidence of distal lesions increases with diabetes and with age. Conditions associated with PAD may be occlusive or functional. Examples of occlusive PAD include peripheral arterial occlusion occlusion, which may be acute, and Buerger's disease (thromboangiitis obliterans), Raynaud's disease, Raynaud's phenomenon and acrocyanosis. Additional non-limiting examples of diseases to be treated include acute and chronic critical limb ischemia, Buerger's disease and critical limb ischemia in diabetes.

As used herein, the term “dermal wound” refers to an injury to the skin in which the skin is cut or broken.

As used herein, the term “promoting angiogenesis” refers to the stimulation of new blood vessels, repairing damaged blood vessels, or increasing the number of blood vessels.

As used herein the terms “culture media” and “culture medium” are used interchangeably and refer to a solid or a liquid substance used to support the growth of cells (e.g., stem cells). Preferably, the culture media as used herein refers to a liquid substance capable of maintaining stem cells in an undifferentiated state. The culture media can be a water-based media which includes a combination of ingredients such as salts, nutrients, minerals, vitamins, amino acids, nucleic acids, proteins such as cytokines, growth factors and hormones, all of which are needed for cell proliferation and are capable of maintaining stem cells in an undifferentiated state. For example, a culture media can be a synthetic culture media such as, for example, minimum essential media α (MEM-α) (HyClone Thermo Scientific, Waltham, Mass., USA), DMEM/F12, GlutaMAX (Life Technologies, Carlsbad, Calif., USA), Neurobasal Medium (Life Technologies, Carlsbad, Calif., USA), KO-DMEM (Life Technologies, Carlsbad, Calif., USA), DMEM/F12 (Life Technologies, Carlsbad, Calif., USA), supplemented with the necessary additives as is further described herein. In some embodiments, the cell culture media can be a mixture of culture media. Preferably, all ingredients included in the culture media of the present disclosure are substantially pure and tissue culture grade. “Conditioned medium” and “conditioned culture medium” are used interchangeably and refer to culture medium that cells have been cultured in for a period of time and wherein the cells release/secrete components (e.g., proteins, cytokines, chemicals, etc.) into the medium.

As used herein, a “bioreactor” refers to a culture system appropriate for supporting growth of cells. In some embodiments, cells may be cultured in a bioreactor system for large-scale growth of surface adherent cells. A non-limiting example of a bioreactor appropriate for practice of the methods disclosed herein is a hollow fiber bioreactor. A hollow fiber bioreactor maximizes the surface area for cells to adhere while minimizing the amount of culture medium needed to support the cells through use of hollow fibers. The hollow fibers are semi-permeable capillary membranes that can be bundled together to create a bioreactor cartridge capable of supporting a high cell density. Methods for use of hollow fiber bioreactors for growth of cells are known in the technical and patent literature, e.g., Sheu et al. “Large-scale production of lentiviral vector in a closed system hollow fiber bioreactor,” Mol. Ther Methods Clin Dev (2015) 2:15020, incorporated by reference in its entirety. Other bioreactors suitable for practice of the disclosed methods include but are not limited to rocking bioreactor systems, stirred tank bioreactor systems, single use bioreactor systems, flow culture bioreactor systems, bioreactors with chambers appropriate for porous cylindrical scaffolds subjected to perfusion culture conditions, and bioreactors with tubular chambers.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, lentiviruses, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

A “viral vector” is defined as a recombinantly produced virus or viral particle that comprises a polynucleotide to be delivered into a cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where modification of the cell is mediated by a lentiviral vector, a vector construct refers to the polynucleotide comprising the lentiviral genome or part thereof, and a therapeutic gene. As used herein, “transfection” or “transduction” in reference to delivery of exogenous nucleic acids carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this invention are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the invention may be based on a genetically or otherwise (e.g., by specific choice of packaging cell system) altered version of a particular retrovirus.

### Cell-Derived Vesicles

Cell-derived vesicles, also referred to as extracellular vesicles, are membrane surrounded structures that are released by cells in vitro and in vivo. Extracellular vesicles can contain proteins, lipids, and nucleic acids and can mediate intercellular communication between different cells, including different cell types, in the body. Two types of extracellular vesicles are exosomes and microvesicles. Exosomes are small lipid-bound, cellularly secreted vesicles that mediate intercellular communication via cell-to-cell transport of proteins and RNA (El Andaloussi, S. et al. (2013) Nature Reviews: Drug Discovery 12(5):347-357). Exosomes range in size from approximately 30 nm to about 200 nm. Exosomes are released from a cell by fusion of multivesicular endosomes (MVE) with the plasma membrane. Microvesicles, on the other hand, are released from a cell upon direct budding from the plasma membrane (PM). Microvesicles are typically larger than exosomes and range from approximately 100 nm to 1 μm.

### Cells

Cell-derived vesicles (e.g., exosomes and/or microvesicles) can be isolated from eukaryotic cells. Non-limiting examples of cells that cell-derived vesicles can be isolated from include stem cells. Non-limiting examples of such stem cells include adult stem cells, embryonic stem cells, embryonic-like stem cells, neural stem cells, or induced pluripotent stem cells. In some embodiments, the stem cell is an adult stem cell that is optionally a mesenchymal stem cell. In one aspect the stem cell, e.g., the mesenchymal stem cells, has been cultured under conditions of hypoxia and low serum or serum-free conditions.

The cells of the present disclosure may be modified, for example, by genetic modification. In some embodiments, the cells are modified to express at least one exogenous nucleic acid and/or at least one exogenous protein. In some embodiments, the cells are modified to express at least one endogenous nucleic acid and/or at least one endogenous protein. The modification may be a transient modification. In other embodiments, the modification may be a stable modification. It is contemplated that by modifying the cells prior to collection of the cell-derived vesicles released by the modified cells, one can collect exosomes containing different amounts and types of proteins, lipids, and nucleic acids as compared to unmodified cells. Any method for cellular modification known to one of skill in the art can be used to modify the cells.

In some embodiments, the cells of the present disclosure are modified to express at least one exogenous or endogenous nucleic acid and/or at least one exogenous or endogenous protein. Non-limiting examples of nucleic acids include one or more or all of DNA and RNA, for example, a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers.

In some embodiments the exogenous or endogenous nucleic acid encodes a micro RNA (miRNA), for example, miR-150 (GenBank Accession No: NR_029703.1 (SEQ ID NO: 1)), miR-126 (GenBank Accession No: NR_029695.1 (SEQ ID NO: 2)), miR-132 (GenBank Accession No: NR_029674.1 (SEQ ID NO: 17)) miR-296 (GenBank Accession No: NR_029844.1 (SEQ ID NO: 3)), let-7 (GenBank Accession No: NR_029695.1 (SEQ ID NO: 4)), and equivalents thereof. In some embodiments the exogenous or endogenous protein is platelet derived growth factor receptor (PDGFR), wherein the PDGF is expressed by a transgene encoding PDGF (e.g., PDGFR-A (GenBank Accession No: NM_006206.4 (SEQ ID NO: 5)), PDGFR-B (GenBank Accession No: NM_002609.3 (SEQ ID NO: 6), or equivalents thereof). In some embodiments the exogenous protein is Collagen, Type 1, Alpha 2 (COL1A2), (GenBank Accession No: NM_000089.3 (SEQ ID NO: 7), or equivalents thereof). In some embodiments the exogenous or endogenous protein is Collagen, Type VI, Alpha 3 (COL6A3), (GenBank Accession No: NM_004369.3 (SEQ ID NO: 8), or equivalents thereof). In some embodiments the exogenous protein is EGF-like repeats- and discoidin i-like domains-containing protein 3 (EDIL3), (GenBank Accession No: NM_005711.4 (SEQ ID NO: 9), or equivalents thereof. In some embodiments the exogenous or endogenous protein is epidermal growth factor receptor (EGFR) (GenBank Accession No: NM_005228.3 (SEQ ID NO: 10), or equivalents thereof. In some embodiments the exogenous protein or endogenous is fibroblast growth factor receptor (FGF) (GenBank Accession No: M60485.1 (SEQ ID NO: 11), or equivalents thereof. In some embodiments the exogenous or endogenous protein is fibronectin (FN1) (GenBank Accession No: M10905.1 (SEQ ID NO: 12), or equivalents thereof. In some embodiments the exogenous or endogenous protein is Milk fat globule-EGF factor 8 (MFGE8) (GenBank Accession No: NM_005928 (SEQ ID NO: 13), or equivalents thereof. In some embodiments the exogenous or endogenous protein is lectin, galactoside-binding, soluble, 3 binding protein (LGALS3BP) (GenBank Accession No: NM_005567 (SEQ ID NO: 14), or equivalents thereof. In some embodiments the exogenous or endogenous protein is transferrin (TF) (GenBank Accession No: M12530.1 (SEQ ID NO: 15), or equivalents thereof. In some embodiments the exogenous ore endogenous protein is vascular endothelial growth factor (VEGF) (e.g. GenBank X62568.1 and GenBank AY04758) or isoform 165A of VEGF (SEQ ID NO: 19) or equivalents thereof. In some embodiments the exogenous or endogenous protein is vascular endothelial growth factor receptor (VEGFR) (GenBank Accession No: AF063657 (SEQ ID NO: 16), or equivalents thereof. In some embodiments, the cells of the present disclosure do not express exogenous or endogenous VEGF, VEGFR or both. In some embodiments, the cells of the present disclosure are modified to express at least one exogenous or endogenous nucleic acid encoding a protein or an endogenous or exogenous nucleic acid detected in exosomes and/or microvesicles of the present disclosure (and listed in the molecular composition of exosomes section below).

An equivalent or biological equivalent nucleic acid, polynucleotide or oligonucleotide or peptide is one having at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, oligonucleotide or peptide. In alternative embodiment, the equivalent or biological equivalent hybridizes to the reference polynucleotide or oligonucleotide or its complement under conditions of high stringency. In a further aspect, the equivalent or biological equivalent is a peptide encoded by a polynucleotide that hybridizes to the polynucleotide encoding the reference peptide or its complement under conditions of high stringency.

The cells of the present disclosure can be cultured in any culture media known to those of skill in the art. For example, the cell culture media can comprise between 5%-40% fetal bovine serum (FBS), preferably approximately 20% FBS; between 0.5%-5% L-glutamine, preferably approximately 1% L-glutamine; and between 0.5%-1% penicillin and streptomycin (Penn-strep), preferably approximately 1% penn-strep, in a basal media. In some embodiments, at least a portion of the FBS is substituted with a serum replacement, for example, a platelet lysate (e.g., human platelet lysate (hPL)). In some embodiments, the amount of serum replacement (e.g., hPL) in the culture media is between 1%-20%. In some embodiments, the cells are cultured in the absence of FBS. In other embodiments, the cells are cultured in the presence of high levels of serum, for example, 30% serum, 40% serum, 50% serum, or 60% serum.

The cells of the present disclosure can be cultured under any conditions known to those in the field. In some embodiments, the cells of the disclosure are cultured in conditions of about 1-20% oxygen (O₂) and about 5% carbon dioxide (CO₂). In some embodiments, the cells of the present disclosure are cultured under hypoxic or low oxygen conditions (e.g., in the presence of less than 10% O₂). In some embodiments, the hypoxic conditions are between approximately 1% to about 15% CO₂ and between 0.05%-20% oxygen tension. In some embodiments, the cells are cultured under low serum conditions. In some embodiments, the low serum conditions are serum free conditions. In some embodiments, the cells of the present disclosure are cultured at about 37° C. In some embodiments, the cells of the present disclosure can be cultured at about 37° C., 5% CO₂ and 10-20% O₂. In preferred embodiments, the cells of the present disclosure are cultured at about 5% CO₂.

In some embodiments, the cells are cultured in hypoxic conditions for a period of time. For example, the cells may be cultured under hypoxic and low serum conditions for up to about 72 hours prior to vesicle isolation or for up to about 40 hours prior to vesicle isolation. In other embodiments, the cells may be cultured under normoxic conditions for a period of time and then switched to hypoxic conditions and culture for a period of time.

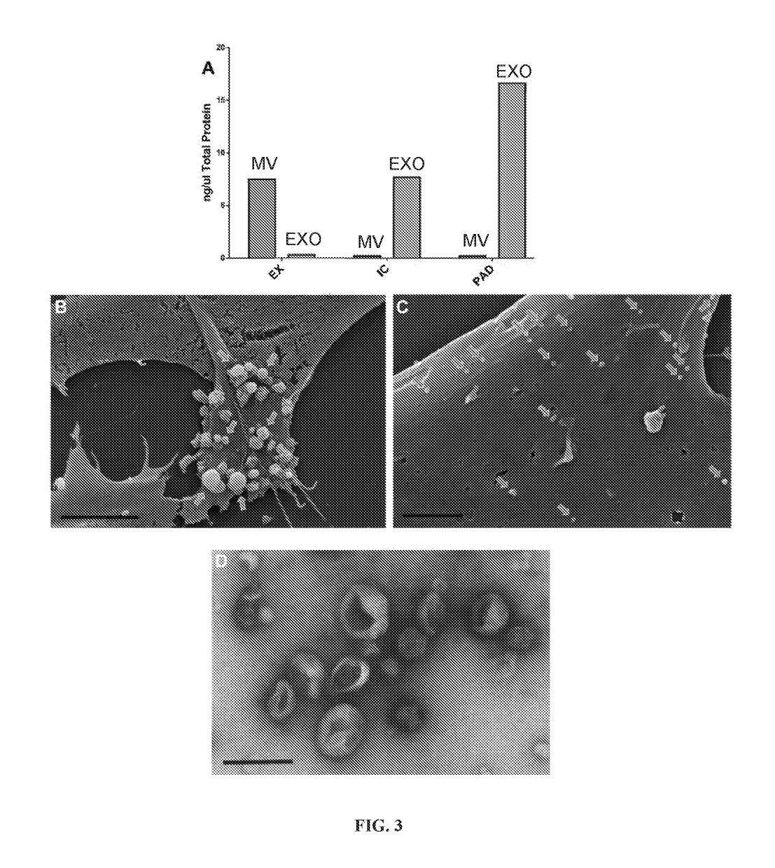

It is surprising that stem cells cultured in hypoxic and/or serum free conditions released more exosomes as compared to conventional culture conditions. See, for example FIG. 3A. It is further surprising that these stressed conditions would produce cell-derived vesicles containing desirable components for use as therapeutics.

### Isolation of Extracellular Vesicles

The purified populations of cell-derived vesicles (e.g., exosomes and/or microvesicles) of the present disclosure can be isolated using any method known by those in the art. Non-limiting examples include differential centrifugation by ultracentrifugation (Théry et al. (2006) Curr. Protoc. Cell Biol. 30:3.22.1-3.22.29; Witmer et al. (2013) J. Extracellular v.2), sucrose gradient purification (Escola et al. (1998) J. Biol. Chem. 273:20121-20127) and combination filtration/concentration (Lamparski et al. (2002) J. Immunol. Methods 270:211-226).

The purified populations of the cell-derived vesicles disclosed herein may be purified from by a method comprising tangential flow filtration (TFF) that may contain a hollow fiber filter or a cartridge filter. In some embodiments, the method for purifying a population of cell-derived vesicles comprises: (a) applying a tangential flow filtration to conditioned media produced by a population of isolated stem cells to isolate an cell-derived vesicle containing fraction; and (b) concentrating the cell-derived vesicle containing fraction to provide a purified population of cell-derived vesicles. In one aspect, the cells are grown under low serum and hypoxic or low oxygen conditions for a period of time prior to collecting the conditioned media from the cell population.

In some embodiments, after step (a) cell debris and other contaminates are removed from the cell-derived vesicle containing fraction prior to step (b).

In some embodiments, the population of stem cells were cultured under hypoxic and low serum conditions for up to about 72 hours prior to performing step (a). In some embodiments, the hypoxic conditions are between approximately 1%-15% CO₂ and between 0.05%-20% oxygen tension. In some embodiments, the low serum conditions are serum free conditions.

The isolated stem cells used for the methods described herein can be any stem cell known to those of skill in the art. Non-limiting examples of stem cells include adult stem cells, embryonic stem cells, embryonic-like stem cells, neural stem cells, or induced pluripotent stem cells. In some embodiments, the stem cells are mesenchymal stem cells.

The tangential flow filtration unit can be between about 50 kilodalton and about 400 kilodalton nominal molecular weight limit filtration unit. For example, the tangential flow filtration unit is about a 100 kilodalton nominal molecular weight limit filtration unit or about a 300 kilodalton nominal molecular weight limit filtration unit (e.g., Minimate™ Tangential Flow Filtration Capsules (Pall Corporation, Port Washington, N.Y., USA) and Pellicon Ultrafiltration Cassettes (EMD Millipore, Billerica, Mass., USA)). In some embodiments, step (a) of the method disclosed herein is performed using an approximately 200 nanometer filter.

In some embodiments, step (b) of the method disclosed herein is performed using a filtration device. For example, the filtration device may be an approximately 100 kilodalton nominal molecular weight limit filtration device or an approximately 300 kilodalton nominal molecular weight limit filtration device.

In some embodiments, the purified populations of cell-derived vesicles (e.g., exosomes and/or microvesicles) of the present disclosure can be isolated from conditioned media via direct isolation using membrane filtration devices (e.g. VivaSpin Centrifugal Concentrator, (Vivaproducts, Inc. Littleton, Mass., USA)). For example, a 100-300 kDa membrane filtration device used with centrifugal force of 500-6000×g may be used to perform the methods disclosed herein.

In some embodiments, the cells are grown in 20% FBS (or 4% hPL) at atmospheric oxygen percentages (˜21% O₂) for approximately 24-72 hours in order to condition the media. The conditioned media is then precleared by centrifuging at 500×g for 10 minutes. The media can then be cleared again by centrifuging at 2000×g for 15 minutes. Then the sample is centrifuged at 17,000×g for 45 minutes and the resulting pellet is resuspended in a solution (e.g., PBS).

In other embodiments, the cells are grown in 20% FBS (or 4% hPL) at atmospheric oxygen percentages (˜21% O₂) for approximately 24-72 hours in order to condition the media. The conditioned media is then precleared by centrifuging at 500×g for 10 minutes. The media can then be cleared again by centrifuging at 2000×g for 15 minutes. The precleared media can then be placed in a TFF filter with 220 nm cutoff size (equivalent to approximately 2200 kDa) to allow at least a portion of the soluble proteins and smaller cell-derived vesicles to pass through the filter while keeping larger cell-derived vesicles. The cell-derived vesicles can then be washed in a sterile solution (e.g., PBS) to diafiltrate the sample. Then the sample can be further concentrated using a 200 nm filter (e.g., Vivaspin column (Viva Products, Littleton, Mass., USA)).

In some embodiments, microvesicles are isolated from cells cultured in the presence of high levels of serum, for example, 30% serum, 40% serum, 50% serum, or 60% serum. In other embodiments, the microvesicles are isolated from cells cultured in the presence of from about 5% to about 25% serum (e.g., FBS). In some embodiments, at least a portion of the serum is substituted with a serum replacement, for example, a platelet lysate (e.g., human platelet lysate (hPL)). The microvesicles can range in size from about 100 nm to about 1000 nm. The microvesicles can be isolated by any method known to those of skill in the art and, in particular, those described in the present disclosure. In some embodiments, the microvesicles are isolated using tangential flow filtration and filters (e.g., a hollow fiber filtration or a cartridge filter) with size cutoffs to select for a desired microvesicle population, for example, from about 100 nm to about 1000 nm, about 200 nm to about 900 nm, about 300 nm to about 800 nm, about 400 nm to about 700 nm, about 500 nm to about 600. In some embodiments, the filters have a cutoff size of about 100 nm, about 200 nm, about 300 nm, about 400 nm, about 500 nm, about 600 nm, about 700 nm, about 800 nm, about 900 nm, or about 1000 nm.

After isolation, the cell-derived vesicles, e.g., exosomes can be concentrated to provide a purified population of cell-derived vesicles. Any appropriate method can be used to concentrate the cell-derived vesicles, e.g. exosomes. Non-limiting examples of such include centrifugation, ultrafiltration, filtration, differential centrifugation and column filtration with a 100 kDA to 300 kDa pore size, or either a 100 kDA to 300 kDa pore size. Further sub-populations can be isolated using antibodies or other agents that are specific for a specific marker expressed by the desired exosome population.

In some embodiments, the methods disclosed herein further comprise formulating the purified population of cell-derived vesicles by mixing the population with a carrier and/or a therapeutic agent such as a pro-angiogenic agent. Non-limiting examples are suitable carriers are described below. In addition or alternatively, the exosome composition can be combined with trehalose for enhanced stability, e.g., at a concentration of about 15 nM to about 50 nM of trehalose in carrier (e.g., PBS), or alternatively about 25 nM of trehalose in carrier (e.g., PBS). Methods to formulate exosomes with trehalose are described in Bosch et al. (2016) “Trehaolose prevents aggregation of exosomes and cryodamage” Scientific Reports 6, Article number 36162, doe: 10.1038/srep36162, incorporated herein by reference.

### Molecular Composition of Cell-Derived Vesicles

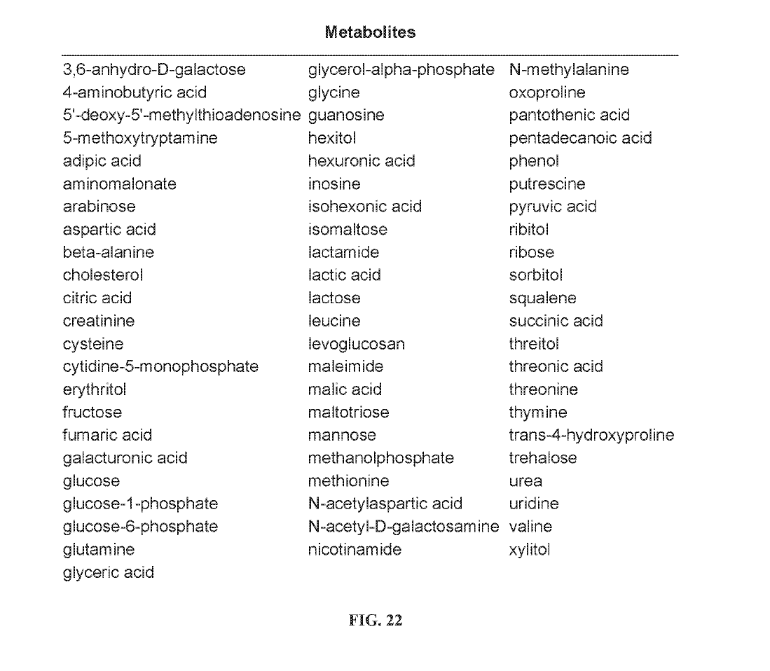

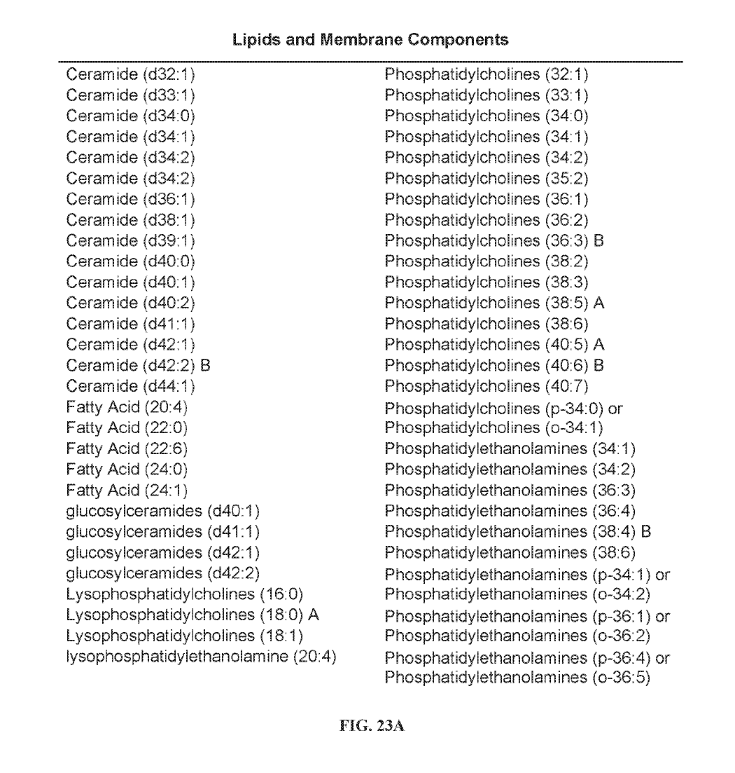

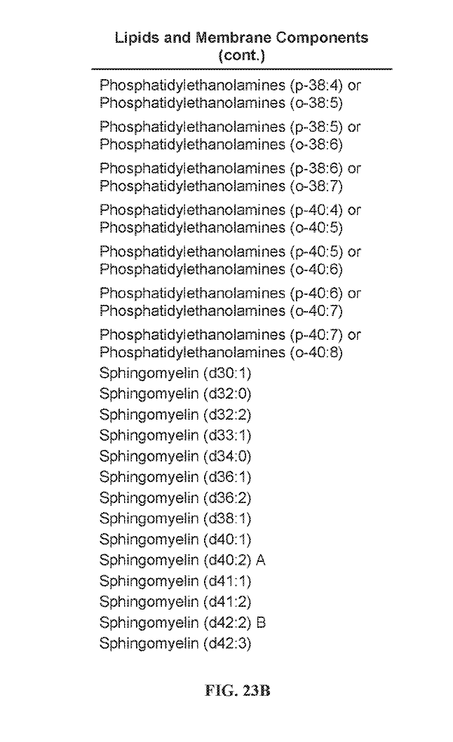

In some embodiments, the purified populations of cell-derived vesicles (e.g., exosomes and/or microvesicles) of the present disclosure comprise proteins, lipids, metabolites, and/or nucleic acids (FIGS. 22-27). In some embodiments, the cell-derived vesicles comprise therapeutic proteins and/or proteins associated with angiogenesis and immune modulation. In some embodiments, the protein content of the purified populations of cell-derived vesicles of the present disclosure is greater than the nucleic acid content of the cell-derived vesicles.

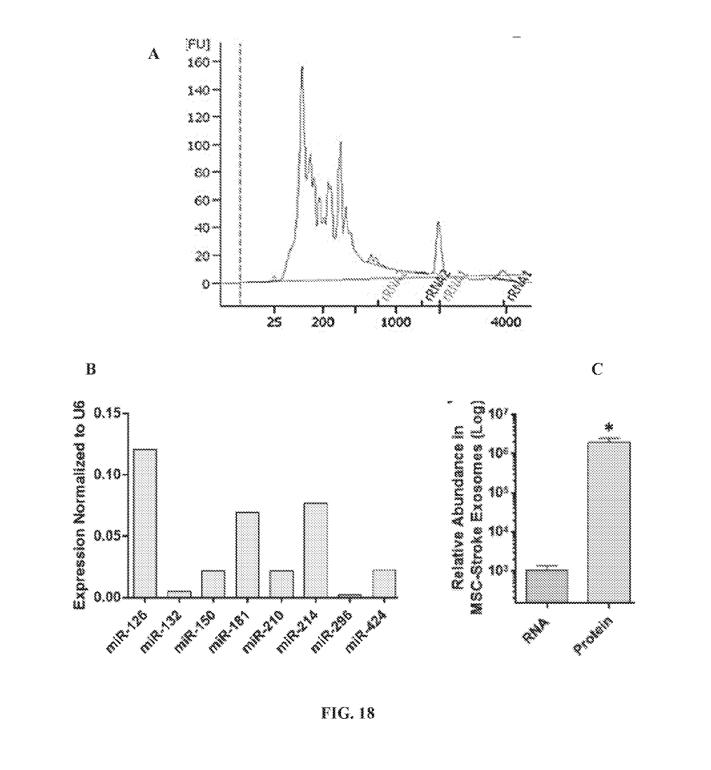

In some embodiments, the purified populations of cell-derived vesicles (e.g., exosomes and/or microvesicles) of the present disclosure may comprise one or more of, or alternatively two or more of, or alternatively three or more of, or alternatively four or more of, or alternatively, five or more of, or alternatively six or more of, all of the following non-limiting examples of exogenous nucleic acids: miR-126, miR-132, miR-150, miR-210, miR-214, miR-296, and miR-424 (see FIG. 18B). Several of the above-listed miRNAs are known in the art to mediate angiogenesis. The above-listed miRNAs were detected in exosomes and/or microvesicles of the present disclosure using a Bioanalyzer and qPCR analyses. Bioanalyzer analysis of exosomes demonstrated enrichment for small RNAs including rRNA2 and rRNA1 (see FIG. 18A).

Surprisingly, the relative abundance of proteins in exosomes and/or microvesicles of the present disclosure was found to far exceed the relative abundance of RNA (see FIG. 18C). This difference in relative abundance was statistically significant. In some embodiments, the relative abundance of protein exceeds the relative abundance of nucleic acids in exosomes and/or microvesicles of the present disclosure.

In some embodiments, the purified populations of cell-derived vesicles (e.g., exosomes and/or microvesicles) of the present disclosure may comprise one or more of, or alternatively two or more of, or alternatively three or more of, or alternatively four or more of, or alternatively, five or more of, or alternatively six or more of, or alternatively seven or more of, or alternatively eight or more of, or alternatively nine or more of, or alternatively ten or more of, or alternatively all of (and integers therebetween) of the following non-limiting examples of metabolites: 3,6-anhydro-D-galactose, 4-aminobutyric acid, 5′-deoxy-5′-methylthioadenosine, 5-methoxytryptamine, s-adenosylmethionine, s-adenosylhomocysteine, adipic acid, aminomalonate, arabinose, aspartic acid, beta-alanine, cholesterol, citric acid, creatinine, cysteine, cytidine-5-monophosphate, erythritol, fructose, fumaric acid, galacturonic acid, glucose, glucose-1-phosphate, glucose-6-phosphate, glutamine, glyceric acid, glycerol-alpha-phosphate, glycine, guanosine, hexitol, hexuronic acid, inosine, isohexonic acid, isomaltose, lactamide, lactic acid, lactose, leucine, levoglucosan, maleimide, malic acid, maltotriose, mannose, methanolphosphate, methionine, N-acetylaspartic acid, N-acetyl-D-galactosamine, nicotinamide, N-methylalanine, oxoproline, pantothenic acid, pentadecanoic acid, phenol, putrescine, pyruvic acid, ribitol, ribose, sorbitol, squalene, succinic acid, threitol, threonic acid, threonine, thymine, trans-4-hydroxyproline, trehalose, urea, uridine, valine, xylitol, and/or the any of the metabolites listed in FIG. 22. The above-listed metabolites were detected in exosomes and/or microvesicles of the present disclosure using an unbiased metabolomics approach. Several of the above-listed metabolites have been shown to modulate gene expression via epigenetic methylation marks on histone tails (e.g. S-adenosylmethionine (SAM) and S-Adenosyl-L-homocysteine (SAH)).

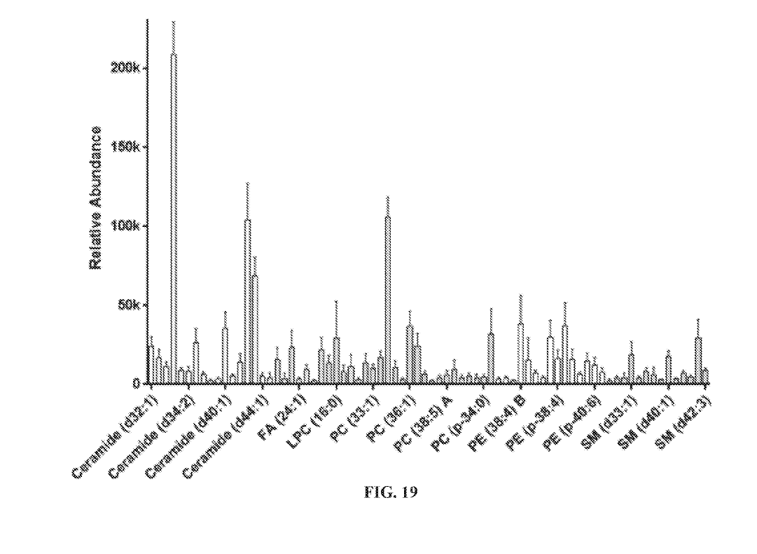

In some embodiments, the purified populations of cell-derived vesicles (e.g., exosomes and/or microvesicles) of the present disclosure may comprise one or more of, or alternatively two or more of, or alternatively three or more of, or alternatively four or more of, or alternatively, five or more of, or alternatively six or more of, or alternatively seven or more of, or alternatively eight or more of, or alternatively nine or more of, or alternatively ten or more of, or alternatively all of (and integers therebetween) of the following non-limiting examples of lipids and membrane components: Ceramide (d32:1), Ceramide (d33:1), Ceramide (d34:0), Ceramide (d34:1), Ceramide (d34:2), Ceramide (d34:2), Ceramide (d36:1), Ceramide (d38:1), Ceramide (d39:1), Ceramide (d40:0), Ceramide (d40:1), Ceramide (d40:2), Ceramide (d41:1), Ceramide (d42:1), Ceramide (d42:2) B, Ceramide (d44:1), Fatty Acid (20:4), Fatty Acid (22:0), Fatty Acid (22:6), Fatty Acid (24:0), Fatty Acid (24:1), glucosylceramides (d40:1), glucosylceramides (d41:1), glucosylceramides (d42:1), glucosylceramides (d42:2), Lysophosphatidylcholines (16:0), Lysophosphatidylcholines (18:0) A, Lysophosphatidylcholines (18:1), lysophosphatidylethanolamine (20:4), Phosphatidylcholines (32:1), Phosphatidylcholines (33:1), Phosphatidylcholines (34:0), Phosphatidylcholines (34:1), Phosphatidylcholines (34:2), Phosphatidylcholines (35:2), Phosphatidylcholines (36:1), Phosphatidylcholines (36:2), Phosphatidylcholines (36:3), Phosphatidylcholines (38:2), Phosphatidylcholines (38:3), Phosphatidylcholines (38:5), Phosphatidylcholines (38:6), Phosphatidylcholines (40:5), Phosphatidylcholines (40:6), Phosphatidylcholines (40:7), Phosphatidylcholines (p-34:0), Phosphatidylcholines (o-34:1), Phosphatidylethanolamines (34:1), Phosphatidylethanolamines (34:2), Phosphatidylethanolamines (36:3), Phosphatidylethanolamines (36:4), Phosphatidylethanolamines (38:4), B Phosphatidylethanolamines (38:6), Phosphatidylethanolamines (p-34:1), Phosphatidylethanolamines (o-34:2), Phosphatidylethanolamines (p-36:1), Phosphatidylethanolamines (o-36:2), Phosphatidylethanolamines (p-36:4), Phosphatidylethanolamines (o-36:5), Phosphatidylethanolamines (p-38:4), Phosphatidylethanolamines (o-38:5), Phosphatidylethanolamines (p-38:5), Phosphatidylethanolamines (o-38:6), Phosphatidylethanolamines (p-38:6), Phosphatidylethanolamines (o-38:7), Phosphatidylethanolamines (p-40:4), Phosphatidylethanolamines (o-40:5), Phosphatidylethanolamines (p-40:5), Phosphatidylethanolamines (o-40:6), Phosphatidylethanolamines (p-40:6), Phosphatidylethanolamines (o-40:7), Phosphatidylethanolamines (p-40:7), Phosphatidylethanolamines (o-40:8), Sphingomyelin (d30:1), Sphingomyelin (d32:0), Sphingomyelin (d32:2), Sphingomyelin (d33:1), Sphingomyelin (d34:0), Sphingomyelin (d36:1), Sphingomyelin (d36:2), Sphingomyelin (d38:1), Sphingomyelin (d40:1), Sphingomyelin (d40:2), Sphingomyelin (d41:1), Sphingomyelin (d41:2), Sphingomyelin (d42:2), B Sphingomyelin (d42:3). The above-listed lipid and membrane components were detected in exosomes and/or microvesicles of the present disclosure using an unbiased lipidomics approach (see FIG. 19 and FIG. 23A-B). Several of the above-listed lipids have been shown to have therapeutic effects in multiple model systems (e.g. sphingomyelin and phosphatidlycholines).

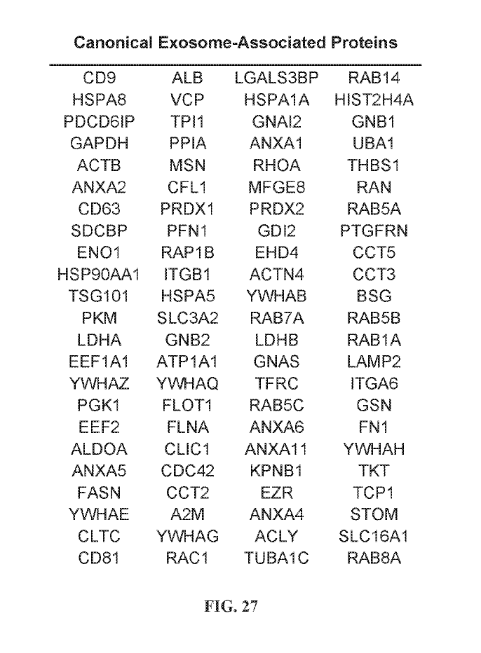

In some embodiments, the purified populations of cell-derived vesicles (e.g., exosomes and/or microvesicles) of the present disclosure may comprise one or more of, or alternatively two or more of, or alternatively three or more of, or alternatively four or more of, or alternatively, five or more of, or alternatively six or more of, or alternatively seven or more of, or alternatively eight or more of, or alternatively nine or more of, or alternatively ten or more of, or alternatively all of (and integers therebetween) of the following non-limiting examples of exosome-associated proteins: CD9, HSPA8, PDCD6IP, GAPDH, ACTB, ANXA2, CD63, SDCBP, ENO1, HSP90AA1, TSG101, PKM, LDHA, EEF1A1, YWHAZ, PGK1, EEF2, ALDOA, ANXA5, FASN, YWHAE, CLTC, CD81, ALB, VCP, TPI1, PPIA, MSN, CFL1, PRDX1, PFN1, RAP1B, ITGB1, HSPA5, SLC3A2, GNB2, ATP1A1, WHAQ, FLOT1, FLNA, CLIC1, CDC42, CCT2, A2M, YWHAG, RAC1, LGALS3BP, HSPA1A, GNAI2, ANXA1, RHOA, MFGE8, PRDX2, GDI2, EHD4, ACTN4, YWHAB, RAB7A, LDHB, GNAS, TFRC, RAB5C, ANXA6, ANXA11, KPNB1, EZR, ANXA4, ACLY, TUBA1C, RAB14, HIST2H4A, GNB1, UBA1, THBS1, RAN, RAB5A, PTGFRN, CCT5, CCT3, BSG, RAB5B, RAB1A, LAMP2, ITGA6, GSN, FN1, YWHAH, TKT, TCP1, STOM, SLC16A1, RAB8A, and/or the proteins listed in FIG. 27. The above-listed proteins were detected in exosomes and/or microvesicles of the present disclosure via gas chromatography and mass spectrometry analysis.

In some embodiments, the purified populations of cell-derived vesicles (e.g., exosomes and/or microvesicles) of the present disclosure may comprise one or more of, or alternatively two or more of, or alternatively three or more of, or alternatively four or more of, or alternatively, five or more of, or alternatively six or more of, or alternatively seven or more of, or alternatively eight or more of, or alternatively nine or more of, or alternatively ten or more of, or alternatively all of (and integers therebetween) of the following non-limiting examples of distinctive proteins which include proteins not previously associated with exosome identity: FN1, EDIL3, TF, ITGB1, VCAN, ANXA2, MFGE8, TGB1, TGFB2, TGFBR1, TGBFR2, TGFBI, TGFBRAP1, BASP1, COL1, COL6, GAPDH, ITGA3, FBN1, ITGAV, ITGB5, NOTCH2, SDCBP, HSPA2, HSPA8, NT5E, MRGPRF, RTN4, NEFM, INA, NRP1, HSPA9, FBN1, BSG, PRPH, FBLN1, PARP4, FLNA, YBX1, EVA1B, ADAM10, HSPG2, MCAM, POSTN, GNB2, GNB1, ANPEP, ADAM9, ATP1A1, CSPG4, EHD2, PXDN, SERPINE2, CAV1, PKM, GNB4, NPTN, CCT2, LGALS3BP, and MVP. The above-listed proteins were detected in exosomes and/or microvesicles of the present disclosure via gas chromatography and mass spectrometry analysis.

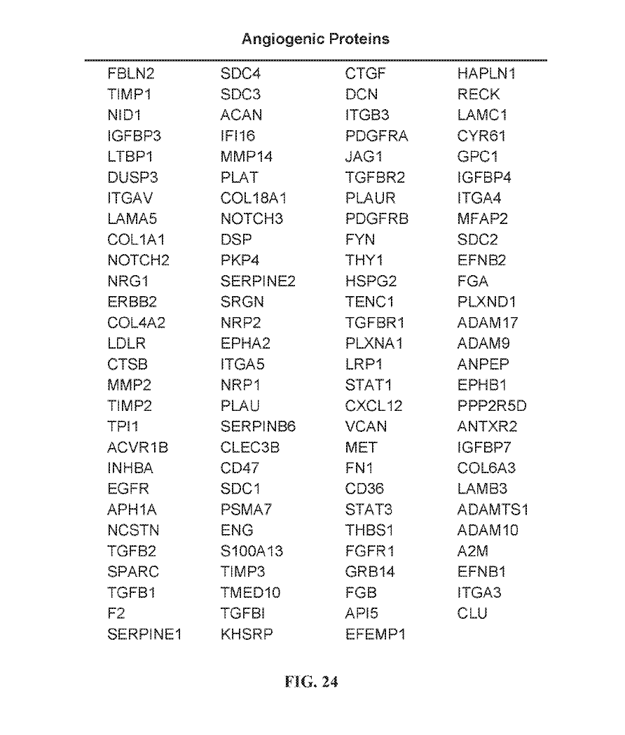

In some embodiments, the purified populations of cell-derived vesicles (e.g., exosomes and/or microvesicles) of the present disclosure may comprise one or more of, or alternatively two or more of, or alternatively three or more of, or alternatively four or more of, or alternatively, five or more of, or alternatively six or more of, or alternatively seven or more of, or alternatively eight or more of, or alternatively nine or more of, or alternatively ten or more of, or alternatively all of (and integers therebetween) of the following non-limiting examples of proteins associated with angiogenesis: FBLN2, TIMP1, NID1, IGFBP3, LTBP1, DUSP3, ITGAV, LAMA5, COL1A1, NOTCH2, NRG1, ERBB2, COL4A2, LDLR, TSB, MMP2, TIMP2, TPI1, ACVR1B, INHBA, EGFR, APH1A, NCSTN, TGFB2, SPARC, TGFB1, F2, SERPINE1, SDC4, SDC3, ACAN, IFI16, MMP14, PLAT, COL18A1, NOTCH3, DSP, PKP4, SERPINE2, SRGN, NRP2, EPHA2, ITGA5, NRP1, PLAU, SERPINB6, CLEC3B, CD47, SDC1, PSMA7, ENG, S100A13, TIMP3, TMED10, TGFBI, CTGF, DCN, ITGB3, PDGFRA, JAG1, TGFBR2, PLAUR, PDGFRB, FYN, THY1, HSPG2, TENC1, TGFBR1, PLXNA1, LRP1, STAT1, CXCL12, VCAN, MET, FN1, CD36, STAT3, THBS1, FGFR1, GRB14, FGB, API5, HAPLN1, RECK, LAMC1, CYR61, GPC1, IGFBP4, ITGA4, MFAP2, SDC2, EFNB2, FGA, PLXND1, ADAM17, ADAM9, ANPEP, EPHB1, PPP2R5D, ANTXR2, IGFBP7, COL6A3, LAMB3, ADAMTS1, ADAM10, A2M, EFNB1, ITGA3, CLU, KHSRP, and EFEMP1 (FIG. 24). The above-listed proteins were detected in exosomes and/or microvesicles of the present disclosure via gas chromatography and mass spectrometry analysis.

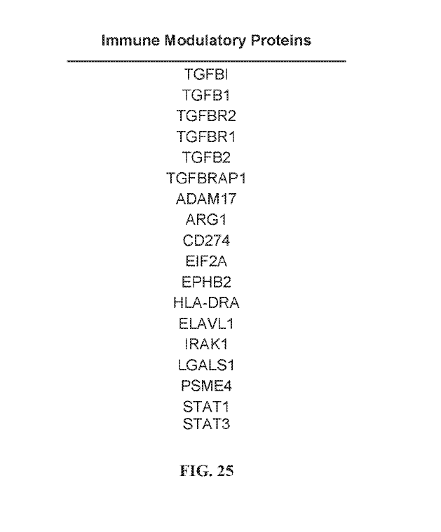

In some embodiments, the purified populations of cell-derived vesicles (e.g., exosomes and/or microvesicles) of the present disclosure may comprise one or more of, or alternatively two or more of, or alternatively three or more of, or alternatively four or more of, or alternatively, five or more of, or alternatively six or more of, or alternatively seven or more of, or alternatively eight or more of, or alternatively nine or more of, or alternatively ten or more of, or alternatively all of (and integers therebetween) of the following non-limiting examples of proteins associated with immune modulation: TGFBI, TGFB1, TGFBR2, TGFBR1, TGFB2, TGFBRAP1, ADAM17, ARG1, CD274, EIF2A, EPHB2, HLA-DRA, ELAVL1, IRAK1, LGALS1, PSME4, STAT1, and STAT3 (FIG. 25). The above-listed proteins were detected in exosomes and/or microvesicles of the present disclosure via gas chromatography and mass spectrometry analysis.

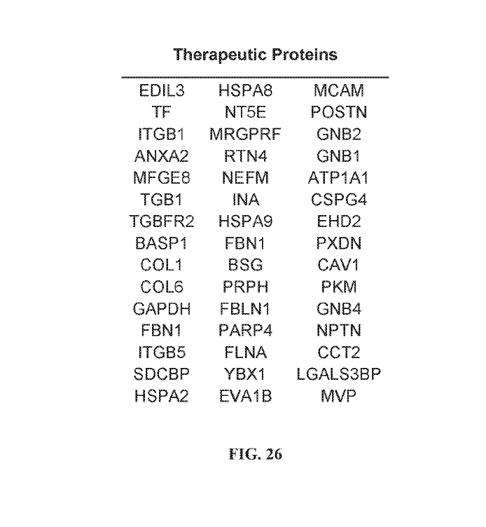

In some embodiments, the purified populations of cell-derived vesicles (e.g., exosomes and/or microvesicles) of the present disclosure may comprise one or more of, or alternatively two or more of, or alternatively three or more of, or alternatively four or more of, or alternatively, five or more of, or alternatively six or more of, or alternatively seven or more of, or alternatively eight or more of, or alternatively nine or more of, or alternatively ten or more of, or alternatively all of (and integers therebetween) of the following non-limiting examples of therapeutic proteins: EDIL3, TF, ITGB1, ANXA2, MFGE8, TGB1, TGBFR2, BASP1, COL1, COL6, GAPDH, FBN1, ITGB5, SDCBP, HSPA2, HSPA8, NT5E, MRGPRF, RTN4, NEFM, INA, HSPA9, FBN1, BSG, PRPH, FBLN1, PARP4, FLNA, YBX1, EVA1B, MCAM, POSTN, GNB2, GNB1, ATP1A1, CSPG4, EHD2, PXDN, CAV1, PKM, GNB4, NPTN, CCT2, LGALS3BP, and MVP (FIG. 26). The above-listed proteins were detected in exosomes and/or microvesicles of the present disclosure via gas chromatography and mass spectrometry analysis.

In further embodiments, the purified populations express one or more combinations of the above.

### Formulations and Pharmaceutical Compositions

The present disclosure provides purified populations of cell-derived vesicles (e.g., exosomes and/or microvesicles). In some embodiments, the population of cell-derived vesicles is substantially homogeneous. In other embodiments, the population of cell-derived vesicles is heterogeneous.

In some embodiments, the substantially homogeneous population is a purified population where at least 90% of the cell-derived vesicles have a diameter of less than 100 nm as determined by a NanoSight LM10HS (available from Malvern Instruments Ltd, Amesbury, Mass., USA).

In some embodiments, the concentration of cell-derived vesicles in the population comprises between about 0.5 micrograms and 100 micrograms of exosome and/or microvesicle protein collected per approximately 10⁶ cells as determined by DC assay (Biorad, Hercules, Calif., USA). In some embodiments, the concentration of cell-derived vesicles in the population comprises between about 100 micrograms and 5000 micrograms of exosome and/or microvesicle protein collected per approximately 10⁶ cells. In other embodiments, the concentration of cell-derived vesicles in the population comprises between about 100 micrograms and 500 micrograms of exosome and/or microvesicle protein collected per approximately 10⁶ cells. In other embodiments, the concentration of cell-derived vesicles in the population comprises between about 500 micrograms and 1000 micrograms of exosome and/or microvesicle protein collected per approximately 10⁶ cells. In other embodiments, the concentration of cell-derived vesicles in the population comprises between about 1000 micrograms and 5000 micrograms of exosome and/or microvesicle protein collected per approximately 10⁶ cells. In other embodiments, the concentration of cell-derived vesicles in the population comprises between about 40 micrograms and 100 micrograms of exosome and/or microvesicle protein collected per approximately 10⁶ cells. In other embodiments, the concentration of cell-derived vesicles in the population comprises less than about 300 micrograms of cell-derived vesicles protein collected per approximately 10⁶ cells. In other embodiments, the concentration of cell-derived vesicles in the population comprises less than about 200 micrograms of cell-derived vesicles protein collected per approximately 10⁶ cells. In other embodiments, the concentration of cell-derived vesicles in the population comprises between about 10 micrograms and 40 micrograms of exosome and/or microvesicle protein collected per approximately 10⁶ cells. In yet other embodiments, the concentration of cell-derived vesicles in the population comprises less than about 30 micrograms of cell-derived vesicles protein collected per approximately 10⁶ cells. In yet other embodiments, the concentration of cell-derived vesicles in the population is less than about 20 micrograms per 10⁶ cells.

The purified populations of cell-derived vesicles can be purified on the basis of average size of the cell-derived vesicles in the composition. Without being bound by theory, it is contemplated that the different sized cell-derived vesicles may contain different types and/or amounts of nucleic acids, protein, lipids, and other components. As such, it is contemplated that compositions comprising cell-derived vesicles of an average size may have a different therapeutic efficacy as compared to a composition comprising cell-derived vesicles of a different average size. In some embodiments, the average diameter of the cell-derived vesicles in the population is between about 0.1 nm and about 1000 nm. In other embodiments, the average diameter of the cell-derived vesicles in the population is between about 2 nm and about 200 nm. In other embodiments, the average diameter of the cell-derived vesicles in the population is less than 100 nm. In yet other embodiments, the average diameter of the cell-derived vesicles in the population is less than 50 nm. In still other embodiments, the average diameter of the cell-derived vesicles in the population is less than about 40 nm.

The compositions disclosed herein may further comprise a carrier, for example, a pharmaceutically acceptable carrier. In some embodiments, more than one pharmaceutically acceptable carrier can be used. Any pharmaceutically acceptable carrier known to those of skill in the art can be used.

In some embodiments, the pharmaceutically acceptable carrier is a preservative, for example, a polymeric preservative or a stabilizing agent.

In some embodiments, the pharmaceutically acceptable carrier is selected from the group consisting of a polyethylene glycol (PEG) (e.g., PEG 150 Distearate), honey, a large molecular weight protein (e.g., bovine serum albumin or soy protein), polyvinyl alcohol, glyceryl monostearate, hyaluronic acid, glycerin, preferably vegetable-derived, proteins, preferably hydrolyzed proteins, (e.g., soy protein and silk protein), vasoline, citrosept, parabens, xanthan gum, i-carregaan, phytagel, Carbopol® polymers, and polyvinyl pyrrolidone.

In some embodiments, exosomes are preserved in serum albumin. Non-limiting examples of serum albumins appropriate for preservation of exosomes include bovine serum albumin (BSA), human serum albumin (HSA), ovalbumin (OVA), and lactalbumin.

Biocompatible gelation agents include thermosensitive sol-gel reversible hydrogels such as aqueous solutions of poloxamers. In one aspect, the poloxamer is a nonionic triblock copolymer composed of a central hydrophobic chain of polyoxypropylene (e.g., (poly(propylene oxide)) flanked by two hydrophilic chains of polyoxyethylene (e.g., poly(ethylene oxide)). In one aspect, poloxamer has the formula

HO(C₂H₄O)_{b}(C₃H₆O)ₐ(C₂H₄O)_{b}OH

wherein a is from 10 to 100, 20 to 80, 25 to 70, or 25 to 70, or from 50 to 70; b is from 5 to 250, 10 to 225, 20 to 200, 50 to 200, 100 to 200, or 150 to 200. In another aspect, the poloxamer has a molecular weight from 2,000 to 15,000, 3,000 to 14,000, or 4,000 to 12,000. Poloxamers useful herein are sold under the tradename Pluronic® manufactured by BASF. Non-limiting examples of poloxamers useful herein include, but are not limited to, Pluronic® F68, P103, P105, P123, F127, and L121.

In one aspect, the biocompatible gelation agent is an agent that is liquid prior to application to a subject (e.g., at room temperature or colder) and becomes a gel after application to the subject (e.g., at body temperature). In one embodiment, the biocompatible gelation agent is a hydrogel.

In another aspect, disclosed herein is a composition comprising exosomes and/or microvesicles and a poloxamer wherein the composition is in a sol (liquid) phase at about 0° C. to about 20° C. and transitions a gel (solid) phase at or near the body temperature or higher, such as about 25° C. to about 40° C., or about 30° C. to about 37° C.

In some aspects, the pharmaceutically acceptable carrier is a pharmaceutically acceptable aqueous carrier such as water or an aqueous carrier. Examples of pharmaceutically acceptable aqueous carrier include sterile water, saline, phosphate buffered saline, aqueous hyaluronic acid, Ringer's solution, dextrose solution, Hank's solution, and other aqueous physiologically balanced salt solutions. In some embodiments, the pharmaceutically acceptable aqueous carrier is Normosol™-R.

Nonaqueous pharmaceutically acceptable carriers include, fixed oils, vegetable oils such as olive oil and sesame oil, triglycerides, propylene glycol, polyethylene glycol, and injectable organic esters such as ethyl oleate can also be used.

Pharmaceutically acceptable carrier can also contain minor amounts of additives, such as substances that enhance isotonicity, chemical stability, or cellular stability. Examples of buffers include phosphate buffer, bicarbonate buffer and Tris buffer, while examples of preservatives include thimerosol, cresols, formalin and benzyl alcohol. In certain aspects, the pH can be modified depending upon the mode of administration. In some aspect, the composition has a pH in the physiological pH range, such as pH 7 to 9.

In one aspect, depending on the type of a pharmaceutically acceptable carrier used, the compositions described herein can comprise about 0.1-100%, 0.1-50%, or 0.1-30%, such as 0.1%, 0.25%, 0.5%, 0.75%, 1%, 2%, 5%, 7%, 10%, 15%, 20%, 25%, 30%, 40%, 45%, 50%, 55%, 60%, 65%, 70%, 75%, 80%, 85%, 90% or 95% of the pharmaceutically acceptable carrier used in the total weight of the composition, or any range between two of the numbers (end point inclusive).

In some embodiments, any one of the above listed pharmaceutically acceptable carriers is expressly excluded.

In some embodiments, the cell-derived vesicles described herein are frozen (e.g., snap-frozen) or freeze-dried (e.g., lyophilized) to promote stability, preserve activity and increase shelf-life. One skilled in the art would understand how to reconstitute the lyophilized product before use.

In some embodiments, the populations of cell-derived vesicles described herein are used immediately after isolation. In other embodiments, the populations of cell-derived vesicles are cryopreserved (e.g. frozen), for example, using any cryopreservation techniques well-known to those skilled in the art. In some embodiments, all or substantially of the cells and/or cellular debris are removed from the culture medium prior to cryopreservation. In some embodiments, all or substantially of the cells and/or cellular debris are removed from the culture medium after cryopreservation.

### Applications and Uses

The populations of cell-derived vesicles described herein can be used in numerous medial applications including for promoting angiogenesis, treating peripheral arterial disease or stroke, and treating a dermal wound in a subject.

The subject may be a mammal, for example, a human or non-human mammals such as a bovine, an ovine, or a porcine. In preferred embodiments, the subject is a human patient. In a further aspect, the subject has been selected for the therapy by diagnostic criteria as determined by the treating physician or health care professional.

In one aspect, provided herein are methods for promoting angiogenesis in a subject in need thereof comprising administering to the subject the purified population or an effective amount of the population and/or a composition described herein. In some embodiments, the subject is administered at least one dose of between approximately 0.1 mg and 200 mg of cell-derived vesicle protein. In other embodiments, the subject is administered at least one dose of approximately 50 mg of cell-derived vesicle protein. In some embodiments, the compositions of cell-derived vesicles are administered prior to or after administration of an isolated stem cell. In other embodiments, the compositions of cell-derived vesicles are administered simultaneously with an isolated stem cell. The compositions herein can be administered to the subject by any method known by those of skill in the art. In some embodiments, the compositions are administered by intravenous injection, direct injection, intramuscular injection, intracranial injection, or topically.

In one aspect, provided herein are methods for treating peripheral arterial disease or stroke in a subject in need thereof comprising administering to the subject the purified population or an effective amount of the population and/or a composition described herein. In some embodiments, the subject is administered at least one dose of between approximately 0.1 mg and 200 mg of cell-derived vesicle protein. In other embodiments, the subject is administered at least one dose of approximately 50 mg of cell-derived vesicle protein. In some embodiments, the compositions of cell-derived vesicles are administered prior to or after administration of an isolated stem cell. In other embodiments, the compositions of cell-derived vesicles are administered simultaneously with an isolated stem cell. The compositions herein can be administered to the subject by any method known by those of skill in the art. In some embodiments, the compositions are administered by intravenous injection, direct injection, intramuscular injection, intracranial injection, or topically. In some embodiments, the compositions herein can be administered to a subject that has suffered a stroke within 24 hours following the stroke event. In other embodiments, the compositions herein can be administered to a subject that has suffered from a stroke about 24-48 hours following the stroke event. In other embodiments, the compositions herein can be administered to a subject that has suffered a stroke within about 48-72 hours following the stroke event. In other embodiments, compositions herein can be administered to a subject that has suffered a stroke within about 72-96 hours following the stroke event.

In one aspect, provided herein are methods for treating a dermal wound in a subject in need thereof comprising administering to the subject the purified population or an effective amount of the population and/or a composition described herein. In some embodiments, the subject is administered at least one dose of between approximately 0.1 mg and 200 mg of cell-derived vesicle protein. In other embodiments, the subject is administered at least one dose of approximately 50 mg of cell-derived vesicle protein. In some embodiments, the compositions of cell-derived vesicles are administered prior to or after administration of an isolated stem cell. In other embodiments, the compositions of cell-derived vesicles are administered simultaneously with an isolated stem cell. The compositions herein can be administered to the subject by any method known by those of skill in the art. In some embodiments, the compositions are administered by intravenous injection, direct injection, intramuscular injection, intracranial injection, or topically.

### Kits

The agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In some embodiments, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In some embodiments, the preservation solution contains an amount of metalloprotease inhibitors.

As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to a subject, such as a syringe, topical application devices, or IV needle tubing and bag.

The therapies as describe herein can be combined with appropriate diagnostic techniques to identify and select patients for the therapy. For example, an ankle-brachial index (ABI) test may be performed to compare blood pressure in a patient's ankle from blood pressure in the patient's arm or Doppler ultrasound may look for blood flow in the major arteries and veins in the limbs. Thus, patients harboring the mutation can be identified prior to symptoms appearing or before advancement of the disease.

The following examples are provided to illustrate and not limit the disclosure.

### EXAMPLES

Bone marrow derived mesenchymal stem cells (MSCs) exhibit tissue healing capabilities via signaling to endogenous cell populations including immune cells and endothelial cells (Meyerrose, T. et al. (2010) Advanced Drug Delivery Reviews 62(12): 1167-1174). MSCs have also shown promise as a potential therapeutic for PAD through the secretion of a robust profile of angiogenic signaling proteins, however, it remains unclear which factors are the main drivers of MSC induced angiogenesis (Liew, A. et al. (2012) Stem Cell Research & Therapy 3(4):28). Exosomes are small lipid-bound, cellularly secreted vesicles that mediate intercellular communication via cell-to-cell transport of proteins and RNA (El Andaloussi, S. et al. (2013) Nature Reviews. Drug Discovery 12(5):347-357). Interestingly, exosomes have been recently shown to also mediate some of the tissue healing properties of MSCs (Bian, S. et al. (2014) Journal of Molecular Medicine 92(4):387-397; Kordelas, L. et al. (2014) Leukemia 8(4):970-973; Zhang, B. et al. (2014) Stem Cells 33(7):2158-2168), however, the underlying mechanisms by which MSC derived exosomes exert their tissue healing properties remain unclear.

Additionally, the angiogenic potential of MSCs can vary due to differences in their microenvironment (Rosova, I. et al. (2008) Stem Cells 26(8):2173-2182). MSCs are generally expanded in high serum (10-20%) containing media under atmospheric oxygen (normoxic) conditions (21% O₂) prior to injection into animal models (Ikebe, C. et al. (2014) BioMed Research International 2014: 951512). However, MSCs experience a markedly different environmental niche upon injection into tissues affected by PAD, where they are exposed to significantly reduced oxygen tension and a reduced concentration of factors contained in serum due to a lack of proper blood flow (Banfi, A. et al. (2005) Current Atherosclerosis Reports 7(3):227-234). It has been recognized that the angiogenic potential of endothelial cells is enhanced when stimulated under hypoxic conditions (Humar, R. et al. (2002) FASEB Journal: Official Publication of the Federation of American Societies for Experimental Biology 16(8):771-780). Although there is evidence that hypoxic stimulation induces expression of angiogenic signaling proteins in endothelial cells, it is not clear to what extent such changes in the environmental niche affect the MSC proteome (Yamakawa, M. et al. (2003) Circulation Research 93(7):664-673; Beegle, J. et al. (2015) Stem Cells 33(6):1818-1828). Therefore, signaling pathways and gene networks that are differentially expressed at the protein level in MSCs exposed to PAD-like culture conditions as compared to normoxic, high serum expansion conditions were analyzed

As proteins mediate most intracellular activity and communication between cells, mass spectrometry proteomics approaches have been invaluable in elucidating differential cell states and patterns of cellular communication (Johansson, H. J. et al. (2013) Nature Communications 4: 2175). However, mass spectrometry based proteomics approaches have had limitations in depth of analysis, greatly limiting the characterization of signaling proteins within cells as they are often present at low levels as compared to other classes of proteins such as structural proteins, which are present at much higher levels (Hultin-Rosenberg, L. et al. (2013) Molecular & Cellular Proteomics: MCP 12(7):2021-2031). A new mass spectrometry approach, termed high-resolution isoelectric focusing liquid coupled chromatography tandem mass spectrometry (HiRIEF LC-MS/MS), was recently developed and enables deep proteome coverage of cellular lysates (Branca, R. M. et al. (2014) Nature Methods 11(1):59-62). This approach has been demonstrated by Branca et al. to be capable of quantitatively characterizing >10,000 proteins per cell lysate, whereas other methods of mass spectrometry generate datasets with smaller depth of coverage (Branca, R. M. et al. (2014) Nature Methods 11(1):59-62).

The effects of a PAD-like microenvironment on angiogenic signaling protein expression within MSCs and their secreted exosomes were investigated. HiRIEF LC-MS/MS was used to investigate changes in MSC proteomic expression when cultured under normoxic, high serum expansion conditions as compared to conditions that mimic the microenvironment experienced by MSCs upon injection into tissues affected by PAD. It was found that exposure of MSCs to a PAD-like microenvironment increases expression of several pro-angiogenic signaling associated proteins including epithelial growth factor (EGF), fibroblast growth factor (FGF) and platelet derived growth factor (PDGF). In addition, it was observed that exposure of MSCs to a PAD-like microenvironment induces elevated exosome secretion and that these secreted exosomes contain a robust angiogenic signaling profile and are capable of inducing angiogenesis in vitro via the nuclear factor kappa-light-chain enhancer of activated B-cells (NFkB) pathway.

### Example 1

### Material and Methods

### Cell Culture and Reagents

Human bone marrow aspirates from young adult, non-smoking males were obtain from Lonza (Allendale, N.J., USA). For MSC isolation and expansion, bone marrow aspirates were passed through 90 μm pore strainers for isolation of bone spicules. Then, the strained bone marrow aspirates were diluted with equal volume of phosphate-buffered saline (PBS) and centrifuged over Ficoll (GE Healthcare, Waukesha, Wis., USA) for 30 minutes at 700 g. Next, mononuclear cells and bone spicules were plated in plastic culture flasks, using minimum essential media α (MEM-α) (HyClone Thermo Scientific, Waltham, Mass., USA) supplemented with 10% fetal bovine serum (FBS; Atlanta Biologicals, Lawrenceville, Ga., USA) that had been screened for optimal MSC growth. After 2 days, nonadherent cells were removed by 2-3 washing steps with PBS. After passage 2 MSCs were expanded in 20% FBS and MSCs from passages 5-6 were used for experimentation. For serum starvation studies MSCs were washed 3 times with PBS and cultured in exosome isolation media consisting of OptiMEM without phenol red with 1% L-Glut (IC) (Life Technologies, Carlsbad, Calif., USA) for 40 hours. For serum starvation plus low oxygen conditions (PAD) MSC were cultured in exosome isolation media under 1% oxygen tension for 40 hours. Pooled human HUVECS were purchased from Lonza (Allendale, N.J., USA) and cultured according to manufacturer's instructions using EndoGRO-LS Complete media from Millipore (Billerica, Mass., USA).

### Vesicle Isolation and Characterization

MSC were washed 3 times with PBS and switched to exosome isolation media; either 20% FBS media that was pre-cleared of exosomes via 18 hour 120,000×g centrifugation, or OptiMEM (Life Technologies, Carlsbad, Calif., USA) and were conditioned for 40 hours prior to vesicle isolation (Kordelas, L. et al. (2014) Leukemia 8(4):970-973). Microvesicles (MV) were isolated as in previous studies (Witwer, K. W. et al. (2013) Journal of Extracellular Vesicles 2:20360). Briefly conditioned media was cleared of cells and cell debris via centrifugation (500×g and 1000×g respectively), then spun at 17,000×g pellet to isolate MVs. Exosomes were isolated as in previous studies (Witwer, K. W. et al. (2013) Journal of Extracellular Vesicles 2:20360). Briefly, for proteomics studies exosomes were isolated using 0.22 μm filtration to get rid of cells, cell debris and microvesicles prior to being spun at 120,000×g for 2 hours, the pellet was then washed with 39 mLs of PBS and spun again at 120,000×g for 2 hours. All ultracentrifuge steps were performed with a Ti70 rotor in polyallomer quick seal tubes (Beckman Coulter, Brea, Calif., USA). Vesicle concentration was determined using DC (detergent compatible) assay (BioRad, Hercules, Calif., USA) and size distribution assessed using NanoSight LM10HS (Malvern, Amesbury, Mass., USA).

### Electron Microscopy

SEM images were taken with Philips XL30 TMP, (FEI Company, Hillsboro, Oreg., USA Sputter Coater: Pelco Auto Sputter Coater SC-7, (Ted Pella Inc., Redding, Calif. USA). TEM images were taken on Philips CM120 Biotwin Lens, 9 (FEI Company, Hillsboro, Oreg., USA), with 2% uranyl acetate staining using facilities at Electron Microscopy Laboratory, School of Medicine, University of California at Davis.

### Sample Preparation for Proteomics

Cell pellets were lysed with 4% SDS, 25 mM HEPES, 1 mM DTT. EVs were lysed with 2% SDS, 25 mM HEPES, 1 mM DTT. Lysates were heated to 95° C. for 5 min followed by sonication for 1 min and centrifugation, 14,000 g for 15 min. The supernatant was mixed with 1 mM DTT, 8 M urea, 25 mM HEPES, pH 7.6 and transferred to a centrifugation filtering unit, 10 kDa cutoff (Nanosep®, Pall, Port Washington, N.Y., USA), and centrifuged for 15 min, 14.000 g, followed by another addition of the 8 M urea buffer and centrifugation. Proteins were alkylated by 50 mM IAA, in 8 M urea, 25 mM HEPES for 10 min, centrifuged for 15 min, 14.000 g, followed by 2 more additions and centrifugations with 8 M urea, 25 mM HEPES. Trypsin (Promega, Madison, Wis., USA), 1:50, trypsin:protein, was added to the cell lysate in 250 mM urea, 50 mM HEPES and incubated overnight at 37° C. The filter units were centrifuged for 15 min, 14,000 g, followed by another centrifugation with MQ and the flow-through was collected (Branca, R. M. et al. (2014) Nature Methods 11(1):59-62). Peptides from EVs were TMT6 labelled and MSC cells with TMT10 labelled according to manufacturer's instructions (Thermo Fisher Scientific, San Jose, Calif., USA). Peptides were cleaned by a strata-X-C-cartridge (Phenomenex, Torrance, Calif., USA) (Branca, R. M. et al. (2014) Nature Methods 11(1):59-62; Wisniewski, J. R. et al. (2009) Nature Methods 6(5):359-362).

Proteomics on nLC-MS/MS on Thermo Scientific LTQ Orbitrap Velos

Before analysis of exosomes on LTQ-Orbitrap Velos (Thermo Fischer Scientific, San Jose, Calif., USA), peptides were separated using an Agilent 1200 nano-LC system. Samples were trapped on a Zorbax 300SB-C18, and separated on a NTCC-360/100-5-153 (Nikkyo Technos., Ltd, Tokyo, Japan) column using a gradient of A (5% DMSO, 0.1% FA) and B (90% ACN, 5% DMSO, 0.1% FA), ranging from 3% to 40% B in 45 min with a flow of 0.4 μl/min. The LTQ-Orbitrap Velos was operated in a data-dependent manner, selecting 5 precursors for sequential fragmentation by CID and HCD, and analyzed by the linear iontrap and orbitrap, respectively. The survey scan was performed in the Orbitrap at 30.000 resolution (profile mode) from 300-2000 m/z with a max injection time of 500 ms and AGC set to 1×10⁶ ions. For generation of HCD fragmentation spectra, a max ion injection time of 500 ms and AGC of 5×10⁴ were used before fragmentation at 37.5% normalized collision energy. For FTMS MS2 spectra, normal mass range was used, centroiding the data at 7500 resolution. Peptides for CID were accumulated for a max ion injection time of 200 ms and AGC of 3×10⁴, fragmented with 35% collision energy, wideband activation on, activation q 0.25, activation time 10 ms before analysis at normal scan rate and mass range in the linear iontrap. Precursors were isolated with a width of 2 m/z and put on the exclusion list for 60 s. Single and unassigned charge states were rejected from precursor selection.

### Proteomic Data Analysis

GraphPAD Prism was used to calculate differential expression using multiple t-tests and a stringent false discovery cut off of 1% (GraphPAD Prism, La Jolla, Calif., USA). Panther Pathway analysis was used to detect the number of pathways detected in each sample and the number of proteins of each pathway represented in each sample (www.pantherdb.com). Ingenuity Pathway Analysis software was used to analyze enrichment for signaling pathway proteins and putative functionality of proteins present in and between each sample (Qiagen, Redwood City, Calif., USA). ClueGO software was used for gene ontology analysis of each sample to detected broad classes of protein functionality (www.ici.upmc.fr/cluego/cluegoDownload.shtml). CytoScape was used to generate network interactome maps for the angiogenesis interactome of MSCs and exosomes and the NFkB pathway interactome (www.cytoscape.org). The constructed angiome dataset from Chu et al. was used to search for the presence of canonical angiogenesis mediating proteins in data presented herein, with the addition of physically interacting proteins not found in the Chu et al. dataset. The Spike database was used to detect proteins for which there was experimental evidence for physical interactions (i.e., yeast-2-hybrid, co-immunoprecipitation) with the Chu et al. dataset and was accessed via CytoScape.

### Tubule Formation Migration Assay

Primary human umbilical cord vein endothelial cells were purchased from Lonza (Allendale, N.J., USA) and cultured in EndoGRO-LS Complete (Millipore, Billerica, Mass., USA) media as per manufacturer's protocol and plated on growth factor reduced matrigel (Corning, Corning, N.Y., USA) and stained with Calcein AM (Life Technologies, Carlsbad, Calif., USA) and imaged at 16 hours post stimulation at 4× on a Kenyence BZ-9000F (Keyence, Osaka, Japan). EndoGRO basal media was used for control and exosome stimulated wells and EndoGRO-LS Complete was used as a positive control (Millipore, Billerica, Mass., USA). For NFkB inhibitor experiments pyrrolidine dithiocarbamate was used at a concentration of 50 μM.

### Results

### MSCs Exposed to PAD-Like Conditions Show Dynamic Proteomic Changes

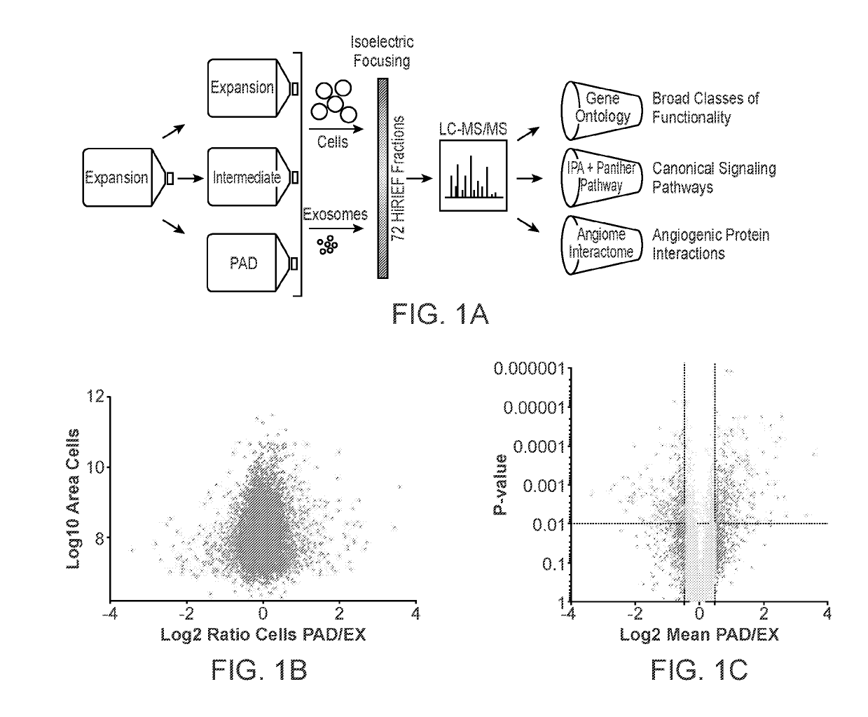

To address what effect PAD-like microenvironment conditions have on the proteomic profile of MSCs, HiRIEF LC/MS-MS was used to quantify the proteome of MSCs. Human MSCs derived from the bone marrow of 3 young adult, non-smoking male donors were cultured under normoxic, high serum expansion conditions until passage 6. After three PBS washes, MSCs were cultured under one of three culture conditions for 40 hours: Normoxic, high serum expansion conditions (EX: 20% FBS, 21% O₂), PAD-like conditions (PAD: 0% FBS, 1% O₂) or an intermediate condition (IC: 0% FBS, 21% O₂) (FIG. 1A).

A total of 6,342 proteins were identified and quantified in each of the 9 MSC samples, with 3 donors for each of the 3 conditions. A total of 580 membrane associated proteins were detected in each of the 9 MSC samples, including canonical MSC surface markers: CD73 (NT5E), CD90 (THY1) and CD105 (ENG) (FIG. 7). The data presented overlaps with and expands beyond the work by Mindaye et al. Statistical analysis of protein expression levels using a false discovery rate of 1% (FDR1%) revealed 315 and 843 differentially expressed proteins respectively between the EX vs IC and EX vs PAD conditions. Analysis of MSC differential expression ratios versus abundance (area) revealed differentially expressed proteins were distributed across the range of abundances of all cellular proteins (FIG. 1). This indicated that the effects of the culture conditions on protein expression were not limited to lowly expressed proteins. Analysis of MSC differential expression ratios versus P-value demonstrated that significantly differentially expressed proteins (FDR1%) were distributed across the range of ratios for all cellular proteins. This indicated that the effects of the culture conditions on protein expression included many new and highly significant findings (FIG. 1).

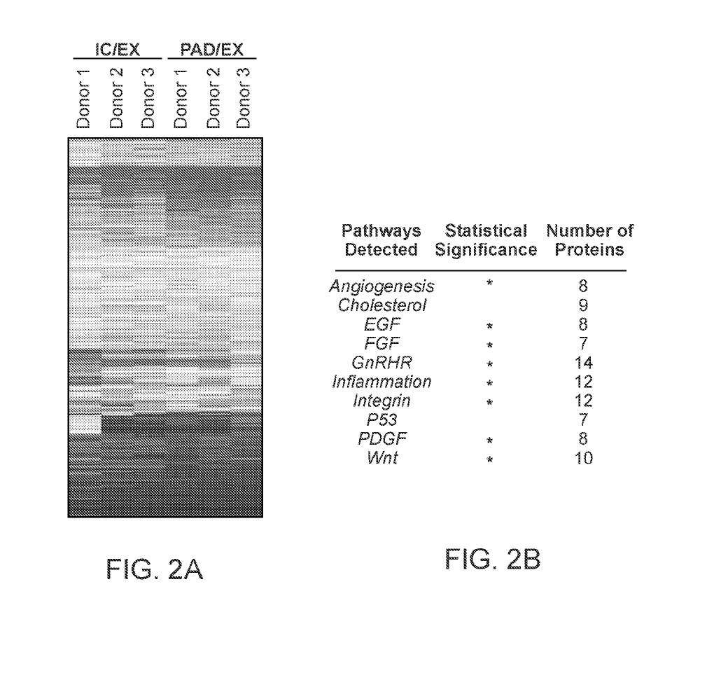

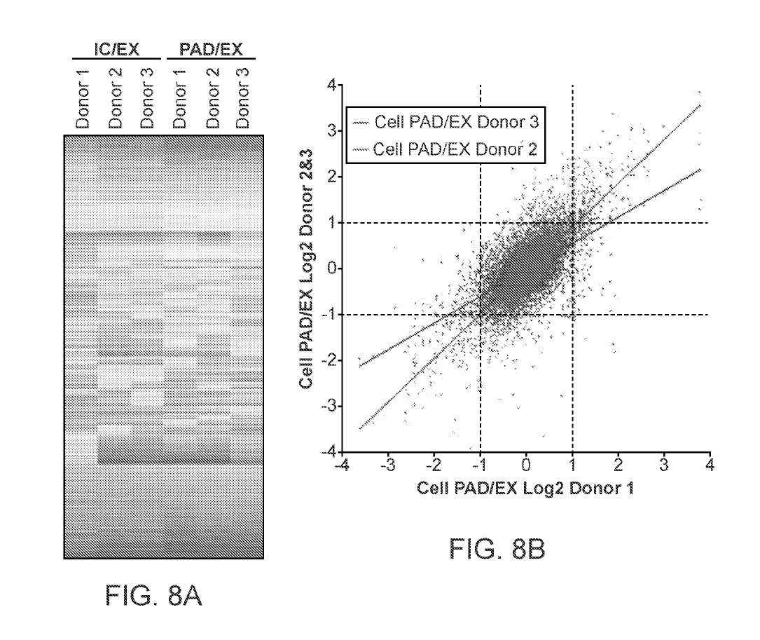

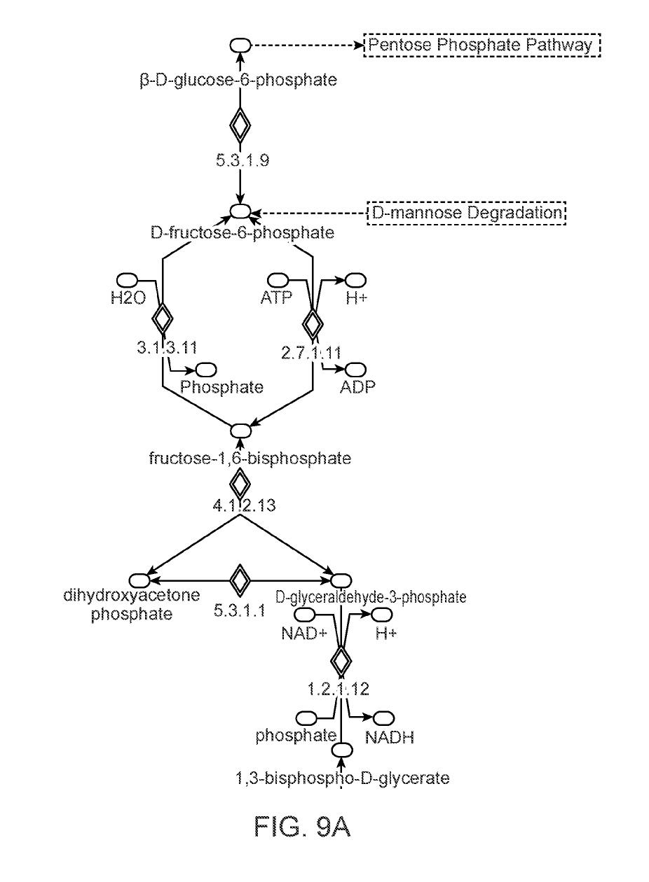

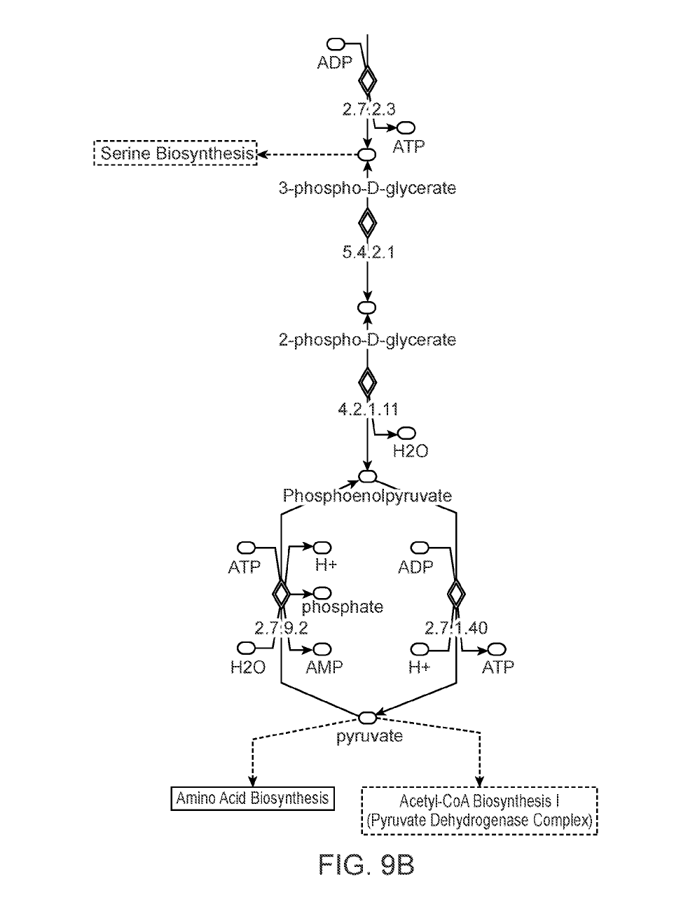

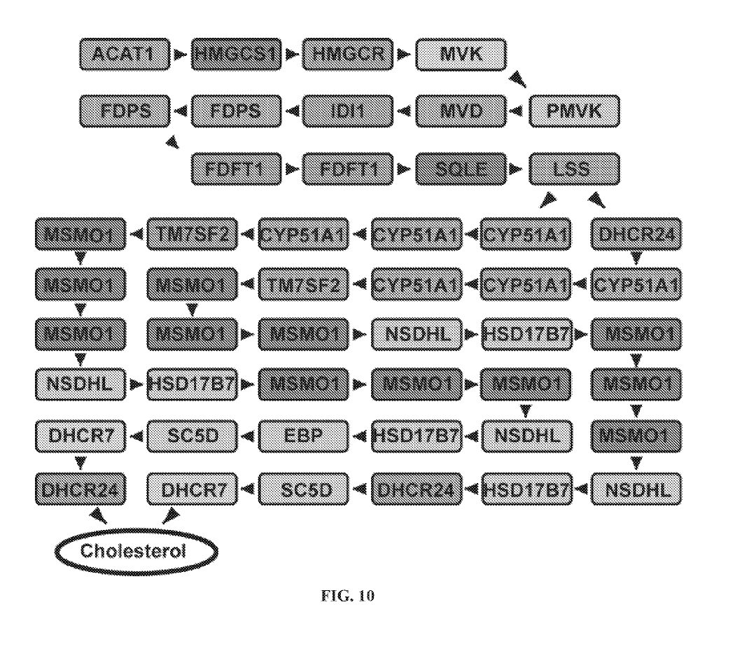

Although global heatmap cluster analysis and linear regression analysis of PAD/EX ratios revealed donor to donor variation in MSCs, it also revealed robust intra-condition concordance between donors (FIGS. 2, 8), especially of significantly differentially expressed proteins. MSCs exposed to PAD-like conditions showed significant increases (FDR1%) in rate limiting proteins of glycolysis (ALDOB, ENO3 and PGK1) and the NRF2/glutathione pathway (ASK1, MKK3/6 and FTH1), which are metabolic and antioxidant associated pathways that have been shown to be modulated with exposure to lower oxygen tension (FIG. 1 and FIG. 9) (Lai, J. C. et al. (1993) Dev Neurosci. 15(3-5):181-193; Hayes, J. D. et al. (2014) Trends Biochem Sci. 39(4): 199-218). Ingenuity Pathway Analysis of differentially expressed cellular proteins (FDR-1%) revealed increased expression of key regulators of the NRF2 pathway, which is the master regulator of glutathione synthesis, in the PAD condition as compared to the EX condition. Analysis was conducted on 3 different donors per condition. For differential expression T-tests with multiple testing correction with an FDR of 1% was used. IC-conditioned MSCs, in contrast, showed no such increases (FDR1%) in glycolysis and glutathione related pathway proteins as compared to the EX condition. Gene ontology analysis using Cytoscape's ClueGO plugin of significantly differentially expressed proteins (FDR1%), revealed numerous cell cycle checkpoint-related pathways (G1 phase, G2/M phase and cytokinesis) involved in the regulation of cellular proliferation were downregulated in both IC and PAD conditions as compared to the EX condition. Ingenuity Pathway Analysis of differentially expressed cellular proteins (FDR-1%) revealed downregulation of proteins involved in proliferation and cell cycle checkpoint-associated pathways, G1 phase progression, G2/M phase progression, cytokinesis, chromosomal segregation in the PAD condition as compared to the EX condition. Cholesterol and lipid biosynthesis pathways were upregulated in both IC and PAD conditions as compared to the EX condition (FIGS. 2 and 10) (Saito, R. et al. (2012) Nature Methods 9(11):1069-1076). Ingenuity Pathway Analysis of differentially expressed cellular proteins (FDR-1%) revealed down regulation of proteins associated with lipid biosynthesis in the PAD condition as compared to the EX condition.

Exposure of MSCs to a PAD-like environment induced significant changes in their proteome. Previous studies have indicated that MSCs are capable of inducing angiogenesis, therefore, Applicants analyzed how this PAD-like microenvironment modulated levels of their angiogenic signaling proteins (Duffy, G. P. et al. (2009) Tissue Eng Part A 15(9):2459-2470; Iwase, H. et al. (2005) Radiat Prot Dosimetry 116(1-4 Pt 2):640-646; Kwon, H. M. et al. (2014) Vascular Pharmacology 63(1): 19-28). To investigate the interaction patterns of known angiogenic proteins in MSCs and to elucidate proteins that physically interact with these known angiogenic proteins, an angiogenesis interactome network map of the MSC proteome was developed. To generate the angiogenesis interactome network map a list of known angiogenic proteins from Chu et al. that were shown to be present in the MSC proteome (Chu, L. H. et al. (2012) Physiol Genomics 44(19):915-924) was derived. CytoScape was then used to include proteins that had experimental evidence of physical interaction with these MSC exosome angiogenic proteins and to show how they interacted with each other (Cline, M. S. et al. (2007) Nat Protoc 2(10):2366-2382). The advantage of this approach is that it not only elucidates the physical interactions of canonical angiogenesis proteins, but additionally reveals other non-canonical proteins that physically interact with the angiome, thereby shedding light on potentially novel mediators of angiogenesis. Analysis of the angiogenesis interactome of proteins present in MSCs across all 3 donors exposed to each of the 3 conditions revealed the most robust clustering of signaling protein interactions was with platelet derived growth factor receptor (PDGFR), epidermal growth factor receptor (EGFR) and NFkB nodes. This indicates that these pathways are likely drivers of MSCs' proangiogenic potential. Furthermore, using Panther Pathway analysis, Applicants found several angiogenic pathways to be significantly (FDR1%) upregulated in MSCs exposed to PAD-like conditions, including canonical angiogenic associated pathways of PDGF, EGF and FGF (FIG. 2) (Mi, H. et al. (2013) Nat Protoc. 8(8):1551-1566). These data collectively demonstrate significantly increased expression of several angiogenic signaling pathways and cholesterol/lipid biosynthesis pathways in MSCs exposed to the PAD condition as compared to the conventional EX condition.

### MSC Exosome Secretion Increases Under PAD-Like Conditions

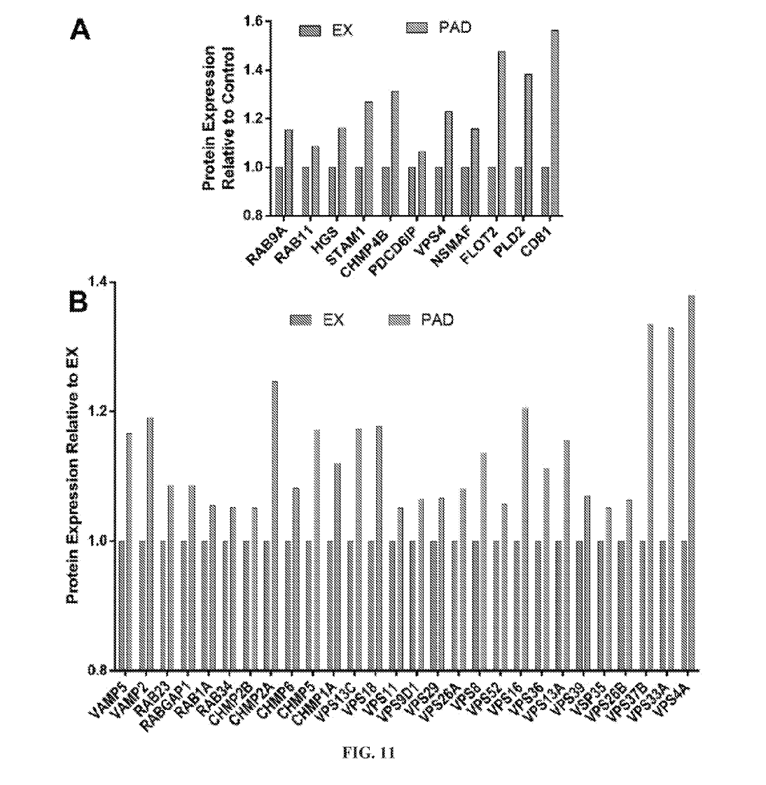

Newly synthesized membranes components such as lipids and cholesterol are transported from their site of genesis at the endoplasmic reticulum to the plasma membrane via vesicular transport (Soccio, R. E. et al. (2004) Arterioscler Thromb Vasc Biol. 24(7): 1150-1160; Lev, S. (2012) Cold Spring Harb Perspect Biol. 4(10)). However, as cells experience decreased rates of proliferation their need for newly synthesized plasma membrane components should also decrease (Baenke, F. et al. (2013) Dis Model Mech. 6(6):1353-1363). Applicants observed that a variety of cell cycle pathways decreased in expression in the IC and PAD conditions as expected, since the cells were exposed to a lower oxygen tension and deprived of growth factor stimulation. Interestingly however, Applicants observed that cholesterol/lipid biosynthesis proteins actually significantly (FDR1%) increased in expression and not decreased, in both IC and PAD conditions as compared to the expansion condition, EX (FIG. 10). This led the Applicants to speculate that an increase in exosome biogenesis could account for the increased expression of proteins involved in cholesterol/lipid biosynthesis. Indeed Applicants observed a trend towards increased expression of proteins involved in the biogenesis of exosomes, prompting us to analyze vesicle secretion of MSCs (FIG. 11).

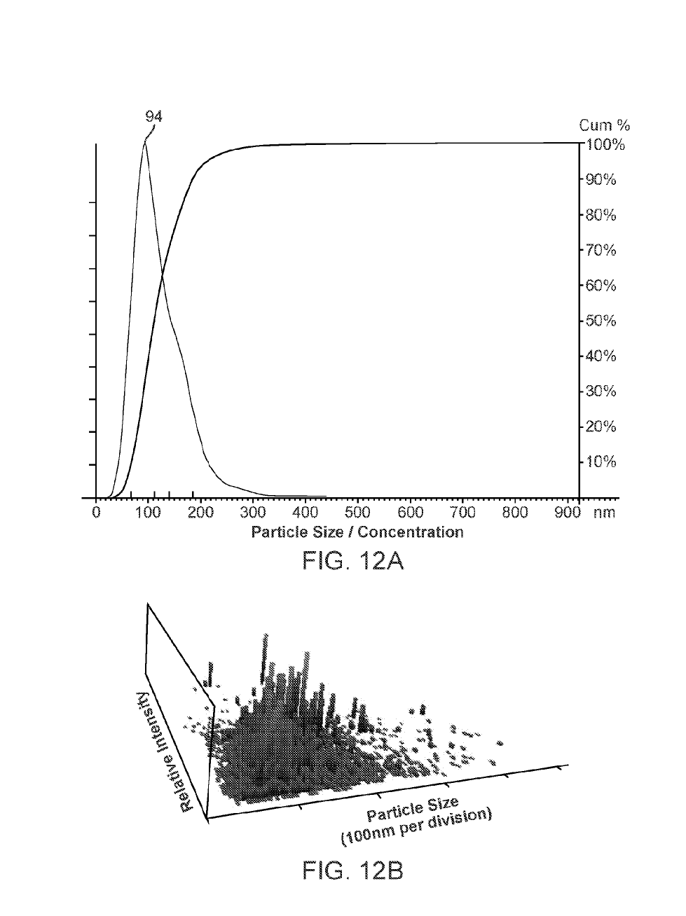

Extracellular vesicles secreted from MSCs (microvesicles, exosomes) were isolated from media that had been conditioned for 40 hours under EX, IC and PAD culture conditions using ultracentrifugation. Analysis of vesicle yield via BCA protein concentration assays revealed that MSC microvesicle secretion decreased whereas exosome secretion substantially increased with MSCs exposed to IC and PAD conditions as compared to EX conditions (FIG. 3). However, exosomes isolated from the EX condition co-isolated with FBS protein from the media. Scanning electron microscopy (SEM) images of MSCs exposed to PAD conditions showed vesicle structures consistent with a decrease in microvesicle secretion and an increase of exosome secretion as compared to MSC exposed to EX conditions (FIG. 3). Furthermore, transmission electron microscopy of isolated PAD-derived MSC exosomes with negative staining is consistent with canonical exosome morphology; additionally, Nanosight analysis revealed that MSC exosomes were of expected size range and MSCs maintained low levels of apoptosis in all conditions (FIGS. 3, 12).

### MSC Exosome Proteome Contains a Robust Profile of Angiogenic Signaling Proteins

As two recent studies demonstrated that MSC exosomes are pro-angiogenic both in vitro and in vivo Applicants used MSC HiRIEF LC-MS/MS to characterize the proteome of MSC derived exosomes from MSCs exposed to IC and PAD conditions (Bian, S. et al. (2014) Journal of Molecular Medicine 92(4):387-397; Zhang, H. C. et al. (2012) Stem Cells and Development 21(18):3289-3297). A total of 1927 proteins were quantified in each of the 6 samples generated from cells derived from 3 donors under both the PAD and IC conditions, 457 of which were not detected in MSCs, indicating exosomal enrichment. Applicants detected 92 of the top 100 most identified exosomal marker proteins from the ExoCarta database in each of Applicants' exosome samples from both conditions, IC and PAD (Simpson, R. J. et al. (2012) Journal of Extracellular Vesicles 1:18374; Mathivanan, S. et al. (2012) Nucleic Acids Research 40(Database issue):D1241-1244; Mathivanan, S. et al. (2009) Proteomics 9(21):4997-5000). Differential expression analysis of exosomes from IC and PAD conditions revealed few significant expression differences (FDR1%) in exosomes between IC and PAD conditions.

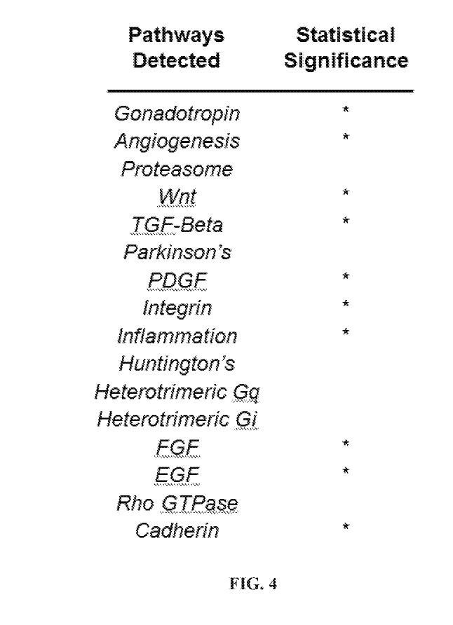

Gene ontology analysis using Cytoscape's ClueGO plugin of the 400 most abundant proteins in the MSC exosome proteome from all 3 donors from both conditions showed representation of vascular and endothelial associated proteins (Bindea, G. et al. (2009) Bioinformatics 25(8): 1091-1093). GO analyses are generally broad based and helpful for a broad overview of the data, but are generally limited in their ability to identify specific signaling pathways. Applicants therefore performed Panther pathway analysis on the MSC exosome proteome and found high representation of several canonical angiogenic associated pathways: cadherin, EGFR, FGF and PDGF (FIG. 4).

Ingenuity Pathway Analysis (IPA) is a robust high throughput data analysis software that is able to predict the induction or inhibition of various cellular activities based on an expert, manually curated database of known protein associations and functions. IPA analysis showed that MSC exosomes contain numerous proteins with a variety of angiogenesis-related functionalities including induction of: angiogenesis, vasculogenesis, cell migration and endothelial cell proliferation.

Next Applicants performed network analysis of the angiogenesis interactome of MSC exosomes, as with the MSC proteome. Applicants showed the most robust representation of protein nodes clustered around the canonical angiogenic pathways of NFKB1/2, Avian Reticuloendotheliosis Viral Oncogene Homolog A (RELA), PDGFRB and EGFR. Furthermore, network analysis of the NFkB pathway showed robust representation of MSC exosome proteins clustering around RELA, NFKB1/2 and TNF-receptor associated factor 6 (TRAF6). These data collectively showed that exosomes derived from MSCs exposed to PAD-like conditions contain a robust profile of angiogenic signaling proteins and putative functionalities closely mirroring those found in MSCs.

### MSC Exosomes Induce Angiogenesis Via the NFkB Pathway in Endothelial Cells

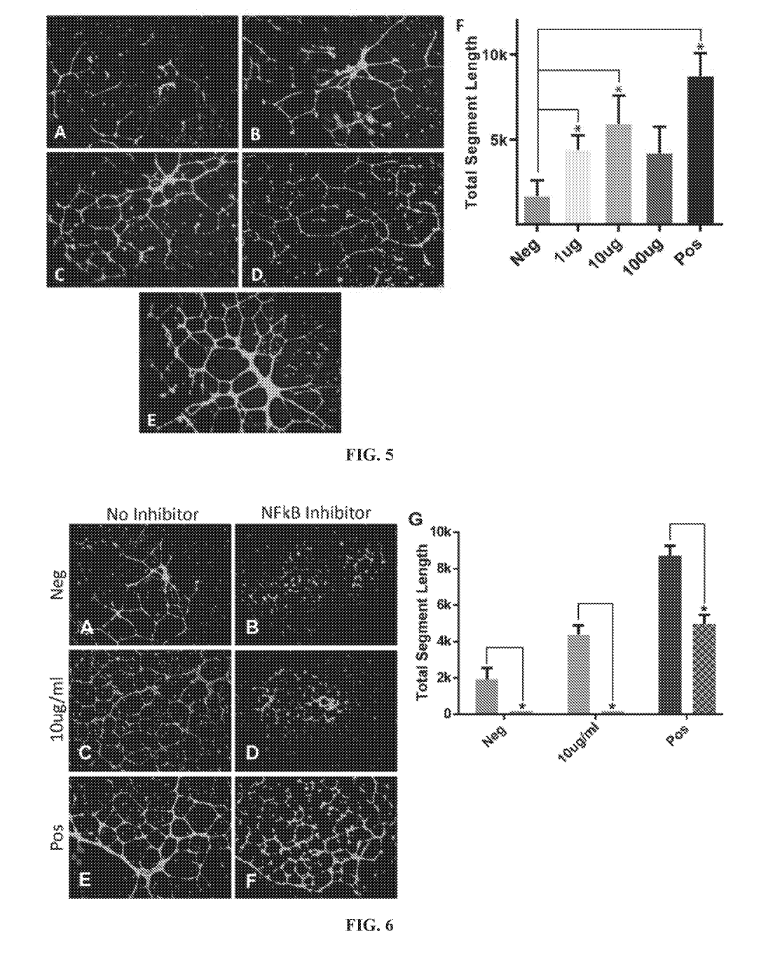

To test the angiogenic potential of MSC exosomes, human umbilical vein endothelial cells (HUVEC) were stimulated in vitro with PAD-derived MSC exosomes. To evaluate their ability to induce tubule formation, a canonical in vitro assay of angiogenesis, was applied. Traditionally, putative therapeutics are known to have a therapeutic index where they behave in a dose dependent manner with decreased effectiveness generally observed at higher doses (Jiang, W. et al. (2015) AAPS J 17(4):891-901). HUVECs were treated with increasing doses of PAD-derived MSC exosomes to test for their effective dose range. The low dose of PAD-derived MSC exosomes (1 μg/mL) induced significant tubule formation compared to the unstimulated control, as did the medium dose (10 μg/mL), measured by total segment length (FIG. 5). However, the high dose of PAD-derived MSC exosomes (100 μg/mL) were less effective than the medium dose indicating the upper limits of the effective dose range (FIG. 5).

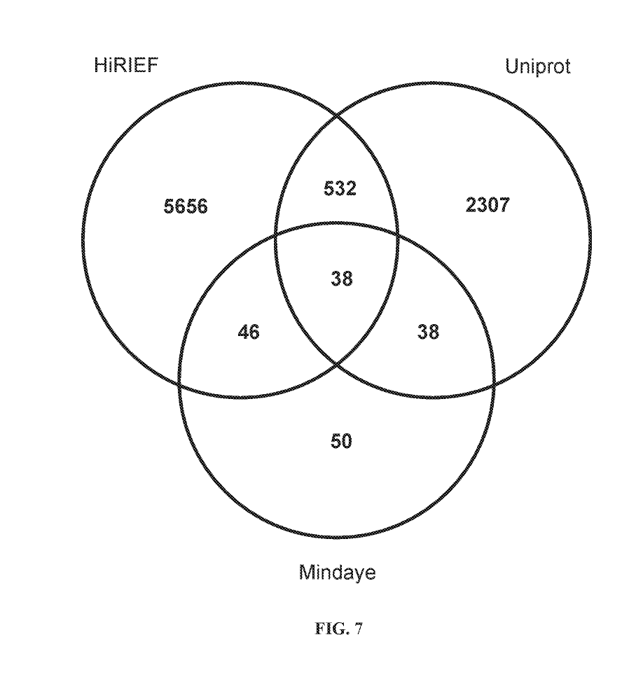

In Applicants' network analysis map of the MSC exosome angiogenesis interactome Applicants observed several hubs of clustering around nodes of the NFkB complex, which is known to mediate angiogenic signaling. Even though these particular nodes, which represent core components of the NFkB complex, were not detected in the MSC exosomes Applicants hypothesized that the presence of numerous NFkB interacting proteins may indicate a potential effector role of this pathway in HUVEC tubule formation. To test this hypothesis HUVECs were treated with pyrrolidine dithiocarbamate (PDTC), a specific inhibitor of NFkB signaling or vehicle control prior to stimulation with PAD-derived MSC exosomes in a tubule formation assay. PAD-derived MSC exosomes induced tubule formation in HUVECs treated with the vehicle control but not in HUVECs treated with PDTC, demonstrating that NFkB signaling is necessary for MSC exosome induction of tubule formation in vitro (FIG. 6). These results indicate that MSC exosomes mediate angiogenesis in a dose dependent manner via the NFkB pathway.

### Discussion

This study presents, to Applicants' knowledge, the most robust proteomic characterization of MSCs and exosomes to date (MSC=6,342 vs 1024, MSC exosome=1927 vs 236) (Kim, H. S. et al. (2012) Journal of Proteome Research 11(2):839-849; Mindaye, S. T. et al. (2013) Stem Cell Research 11(2):793-805). Applicants detected 580 membrane associated proteins including those required to meet the minimal criteria for MSC classification (CD73, CD90, CD105) across all 9 MSC samples, and represents the most robust proteomic profiling of MSC membrane proteins to date (580 vs 172) (Mindaye, S. T. et al. (2013) Journal of Proteomics 78: 1-14). MSCs have been proposed as a therapeutic for PAD, however, the effect of the PAD microenvironment has on both the MSC physiology and MSC induced angiogenesis are poorly understood (Capoccia, B. J. et al. (2009) Blood 113(21):5340-5351). Even though several studies have demonstrated the efficacy of using MSCs for ischemic tissue related diseases, efforts towards identifying the underlying mechanisms of MSC induced angiogenesis have not been robustly investigated, as more focus has been placed on MSC secretion of VEGF and PDGF (Beckermann, B. M. et al. (2008) British Journal of Cancer 99(4):622-631; Deuse, T. et al. (2009) Circulation 120(11 Suppl):S247-S254; Fierro, F. A. et al. (2011) Stem Cells 29(11):1727-1737; Ding, W. et al. (2010) Blood 116(16):2984-2993). The quantitative proteomic methodology Applicants used underscores the need for an unbiased approach which in the present study led to the finding that the MSC proteome is modulated upon exposure to a PAD-like microenvironment and multiple pathways are likely involved in MSC mediated angiogenesis.

Applicants show attenuation of various cell cycle initiation and glycolysis gene networks in MSCs exposed to PAD-like conditions. Network analysis of all 3 donors from all 3 culture conditions (9 samples total) demonstrated that the MSC angiogenesis interactome is enriched for nodes associated with PDGFR, EGFR, and NFkB. This indicated that these known angiogenesis mediating pathways are likely central hubs of intracellular angiogenic signaling within MSCs (Gianni-Barrera, R. et al. (2014) Biochemical Society Transactions 42(6): 1637-1642; Tabernero, J. (2007) Mol Cancer Res. 5(3):203-220; Fujioka, S. et al. (2003) Clin Cancer Res. (1):346-354; Hou, Y. et al. (2008) Dev Dyn 237(10):2926-2935). Furthermore, when MSCs were exposed to PAD-like conditions they significantly increased expression of proteins associated with a subset of angiogenic signaling pathways EGF, FGF, and PDGF.

MSCs are known to mediate much of their tissue healing effects through their secretome in various vascular disease models such as stroke and peripheral arterial disease (Meyerrose, T. et al. (2010) Advanced Drug Delivery Reviews 62(12): 1167-1174; Bronckaers, A. et al. (2014) Pharmacology & Therapeutics 143(2):181-196). Recent studies have demonstrated that a new cell to cell communication system mediated by exosomes is capable of recapitulating much of the beneficial therapeutic effects of MSCs in these disease models (Bian, S. et al. (2014) Journal of Molecular Medicine 92(4):387-397; Kordelas, L. et al. (2014) Leukemia 8(4):970-973; Zhang, B. et al. (2014) Stem Cells 33(7):2158-2168; Lai, R. C. et al. (2010) Stem Cell Research 4(3):214-222). However, the underlying mechanisms by which MSC exosomes modulate these tissue healing effects have yet to be elucidated.

Applicants characterized the proteome of exosomes derived from MSCs exposed to PAD-like conditions (PAD) and the intermediate condition (IC), but not from expansion conditions (EX) since Applicants' HiRIEF LC-MS/MS method requires large quantities of input material and the exosome yield from this condition was too small. Applicants quantitatively characterized 1,927 proteins in MSC exosomes from all three donors across both IC and PAD conditions, of which 457 were not detected in the MSC proteome. A potential explanation for this observed protein enrichment in MSC exosomes is that some proteins can be masked in more complex lysates when using mass spectrometry methodologies, but this does not preclude the possibility that some of these proteins are being directly shuttled into exosomes for secretion (Hultin-Rosenberg, L. et al. (2013) Molecular & Cellular Proteomics: MCP 12(7):2021-2031). Of note is the fact that the proteome of exosomes derived from MSCs appears to lack many canonical secretory signaling proteins such as cytokines and growth factors, but instead contain the downstream mediators of these pathways.

Applicants showed that exosomes from MSCs exposed to PAD-like conditions contain a robust profile of angiogenesis associated proteins that closely mirror the upregulated angiogenic pathways found in MSCs exposed to PAD-like conditions including EGFR, FGF and PDGF pathways. These findings suggest that upon exposure to ischemic tissue conditions attempt to generate a more proangiogenic state via the secretion of exosomes, thereby facilitating localized tissue healing. Further, the main drivers of MSC exosome induced angiogenesis may act via direct signaling to endothelial cell populations or indirectly through inducing chemotaxis of immune cells such as monocytes.

Applicants also showed that proteins mediating cholesterol/lipid biosynthesis and metabolism are significantly upregulated in MSCs that are exposed to PAD-like conditions, while several known exosome biogenesis proteins trend towards increased expression under these same conditions. Numerous cell cycle pathways are significantly downregulated in MSCs exposed to PAD-like conditions and various cell types have substantially lower rates of proliferation when exposed to similar conditions (Rosova, I. et al. (2008) Stem Cells 26(8):2173-2182; Beegle, J. et al. (2015) Stem Cells 33(6):1818-1828). Since, ostensibly there should be much less demand for such high energy cost membrane components and exosomes are known to be enriched for lipid raft components such as cholesterol (Tan, S. S. et al. (2013) Journal of Extracellular Vesicles 2:22614), Applicants therefore speculated that the upregulation of these cholesterol/lipid biosynthesis proteins may be associated with exosome secretion. Applicants showed that MSCs increased secretion of exosomes upon exposure to PAD-like conditions which were of canonical size and morphology. Alternatively the observed increase in lipid biosynthesis may potentially be a cellular adaption to hypoxia in the PAD condition (Masson, N. et al. (2014) Cancer Metab 2(1):3).

Consistent with traditional broad range small molecule dose curves, Applicants show that exosomes derived from MSCs exposed to PAD-like conditions were able to induce angiogenesis in vitro, in a dose dependent manner. MSC exosomes at the highest concentration (100 μg/mL) induced less tubule formation as compared to lower doses, which may indicate an upper limit of the effective dosing range.

Applicants' network analysis indicated that MSC exosomes derived from PAD-like conditions are enriched for several nodes associated with NFkB signaling, which has previously been shown to be an important mediator of angiogenesis (Hou, Y. et al. (2008) Dev Dyn 237(10):2926-2935). Applicants demonstrated that MSC exosome induced angiogenesis is dependent on NFkB signaling, since a specific chemical inhibitor of NFkB signaling completely abrogates the ability of MSC exosomes to induce tubule formation in vitro. It remains unclear, however, to what extent MSC induced angiogenesis can be attributed to exosome mediated effects. Overall, Applicants' data suggest that there are more signaling pathways involved which are worthy of further investigation.

### Conclusion

A common trend that is becoming apparent across the MSC exosome literature is that exosomes derived from MSCs are able to mediate much of the functionality traditionally associated with canonical secretory proteins such as growth factors of the MSC secretome (Bian, S. et al. (2014) Journal of Molecular Medicine 92(4):387-397; Kordelas, L. et al. (2014) Leukemia 8(4):970-973; Zhang, B. et al. (2014) Stem Cells 33(7):2158-2168 Zhang, H. C. et al. (2012) Stem Cells and Development 21(18):3289-3297; Li, T. et al. (2013) Stem Cells and Development 22(6):845-854; Katsuda, T. et al. (2013) Scientific Reports 3: 1197; Lin, S. S. et al. (2014) Neurochem Res. 39(5):922-931; Bruno, S. et al. (2009) Journal of the American Society of Nephrology: JASN 2009; 20(5): 1053-1067; Xin, H. et al. (2013) Stem Cells 31(12):2737-2746). Whether canonical secretory proteins or exosomally delivered proteins are the main drivers of the MSC secretome's functionality still needs further investigation; based on data presented herein it is likely microenvironment dependent.

An exciting open question is whether MSC exosomes derived from PAD-like culture conditions can be used as a therapeutic in lieu of MSCs for a various diseases and if so what the underlying therapeutic mechanisms might be. A study published in 2014 on the first human patient successfully treated with MSC exosomes for graft versus host disease would seem to suggest that this area of research is feasible and worthy of further investigation (Kordelas, L. et al. (2014) Leukemia 8(4):970-973). The data herein suggests that MSC derived exosomes may be a promising therapeutic platform that provides additional benefits to the use of MSCs themselves. The data herein may also provide a blueprint for future studies aiming to attempt to engineer MSC exosomes to be a more efficacious therapeutic for cardiovascular diseases.

### Example 2

### Peripheral Artery Disease

Peripheral artery disease (PAD) of the lower extremities has become a major contributor to the cardiovascular public health burden. It is associated with high rates of morbidity and identifies a cohort of patients that is at increased risk of major cardiovascular ischemic events. PAD is estimated to affect 12% to 15% of people over the age of 65 years, approximately 8-10 million people in the United States. Prevalence is expected to increase significantly as the population ages, becomes more obese, and as diabetes mellitus becomes more common.

PAD is characterized by a lack of proper blood flow to the lower extremities due to narrowing or blockage of arterial vasculature from atherosclerotic plaques. Angioplasty and stent placement are commonly used to treat PAD, however, restenosis and re-occlusion from subsequent blood clot formation and neo-intimal hyperplasia limit the effectiveness of these treatments in many patients.

A potential alternative therapeutic approach to treat PAD is localized induction of angiogenesis to restore blood flow to affected tissues. Studies in animal models of PAD have shown localized induction of angiogenesis via recombinant VEGF therapy. However, this straightforward approach has so far failed to show clear benefits in humans in late-stage clinical trials, perhaps due to the use of a monotherapeutic approach which only targeted a single signaling pathway responsible for one portion of the tissue healing process in PAD (Yla-Herttuala, S. et al. (2007) Journal of the American College of Cardiology 49(10):1015-1026).

Bone marrow derived mesenchymal stem cells (MSCs) promote enhanced tissue healing via signaling to endogenous cell populations including immune cells and endothelial cells. MSCs have shown promise as a potential therapeutic treatment for PAD through the secretion of a diverse profile of angiogenic signaling factors including exosomes. Exosomes are small lipid-bound, cellularly secreted vesicles that mediate intercellular communication via cell-to-cell transport of proteins, RNAs, lipids and metabolites. However, it remains unclear which of these secreted factors are of primary importance in MSC induced angiogenesis. Interestingly, exosomes have been recently shown to also mediate some of the tissue healing properties of MSCs, however, the underlying mechanisms by which MSC exosomes exert their tissue healing properties remain unclear.

The therapeutic application of MSCs in the clinic has advanced faster than the field's understanding of how the cells mediate tissue healing and currently it is not clear how MSC exosomes mediate angiogenesis in models of cardiovascular disease such as PAD. Exosomes are rapidly gaining interest as potential therapeutics for cardiovascular indications, perhaps serving as a safer and potentially more efficacious vehicle to deliver stem cell-derived therapeutics. In addition, the effective engineering of MSC exosomes holds the potential to allow for delivery of novel, therapeutically relevant biologics that have, heretofore, been impractical to deliver clinically, such as miRNA, mRNA, plasmids, membrane and cytosolic proteins.

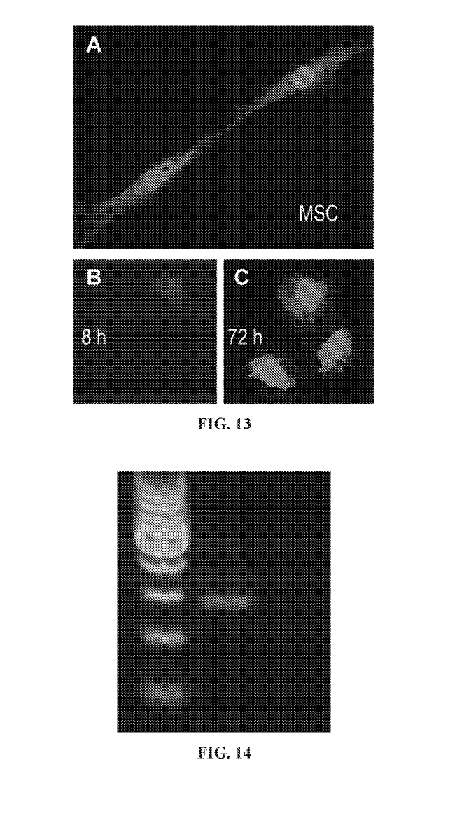

Here, exosomes and microvesicles derived from MSCs were engineered with exogenous biologic components. MSCs were transduced with a lentivirus that overexpressed a fluorescent marker protein, tdTomato, and a miRNA, miR-132. After 16 hours the cells were washed 3×'s and given fresh exosome isolation media (serum free) and placed in hypoxia (1% O2) increases exosome secretion by MSCs. 48 hours later exosomes were isolated and purified from conditioned media using tangential flow filtration. Endothelial cells were then exposed to these isolated exosomes and imaged at 8 and 72 hour timepoints (FIG. 13). Endothelial cells imaged at 8 hours post exosomes exposure show a small amount of fluorescence, indicating delivery of tdTomato on the protein level to cells. However, after 72 hours post exposure endothelial cells show a much higher fluorescent signal indicating additional tdTomato proteins translated from functional tdTomato mRNAs delivered via exosomes.

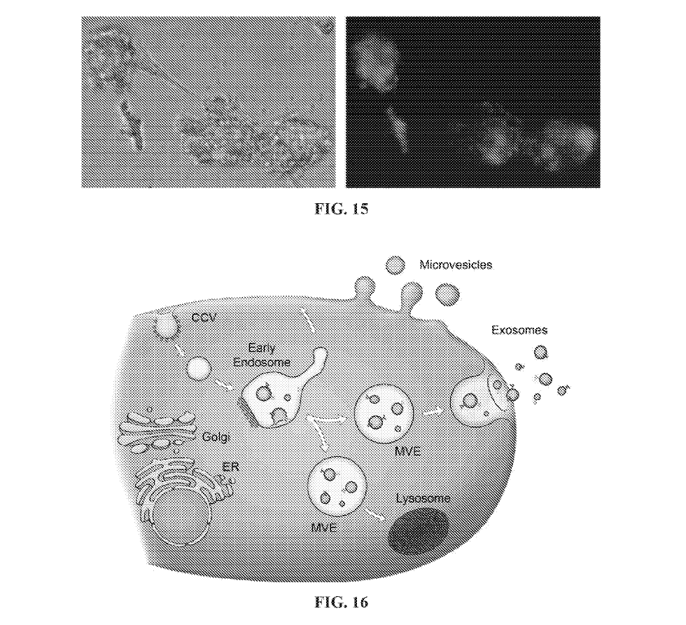

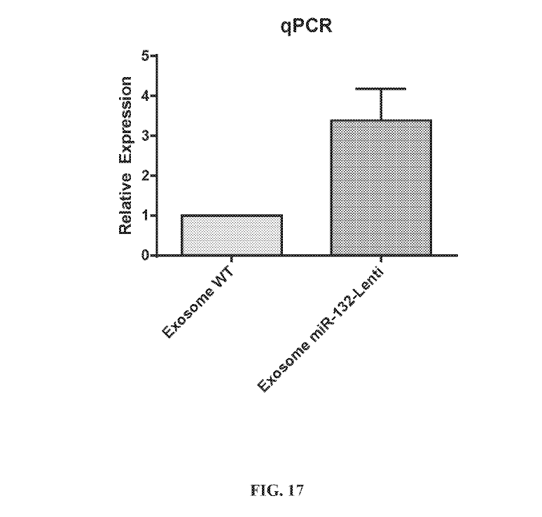

In a separate experiment, MSCs were transfected with a plasmid expression vector overexpressing miR-132 and tdTomato (SEQ ID NO: 18). After 16 hours the cells were washed 3×'s and given fresh microvesicle isolation media. Microvesicles were harvested from media that had been conditioned for 48 hours using ultracentrifugation. DNA was isolated from purified microvesicles and PCR demonstrated the presence of the expression plasmid (FIG. 14). The data herein demonstrate that these microvesicles delivered functional plasmids expressing tdTomato and miR-132 to endothelial cells as detected by fluorescence microscopy at 48 hours post exposure (FIG. 15).

### Example 3

### Large Scale Manufacturing Using a Hollow Fiber Reactor

A hollow fiber bioreactor may be used to scale up production of exosomes and/or microvesicles. This method reduces personnel labor and media usage, both of which can be costly expenditures. In this example, a hollow fiber cartridge was coated with an extracellular matrix (ECM) protein coating. Non-limiting examples of appropriate ECM and other coatings also appropriate for use with this method include fibronectin, gelatin, vitronectin, matrigel, and collagen. 10-100 million stem cells were seeded onto the coated hollow fiber cartridge. Cells were grown in expansion media: 5-20% FBS in basal media with 0-5% L-Glut, with a gas mixture of 20% oxygen, 5% C02, and 75% nitrogen. Alternatively, cells may be cultured at lower percentages of oxygen (between 1% and 20%), with C02 at 5%. Following several days of cell expansion, the media is switched to isolation media, basal media with 0-5% L-Glut, with a gas mixture of 1-20% oxygen, 5% C02 with the balance being nitrogen. After 15-96 hours, exosomes and/or microvesicles are harvested from the resulting conditioned media. Exosomes and/or microvesicles may be isolated from the conditioned media either by TFF or by direct isolation using 100-300 kDa membrane filtration devices (e.g. VivaSpin) using centrifugal force of 500-6000×g.

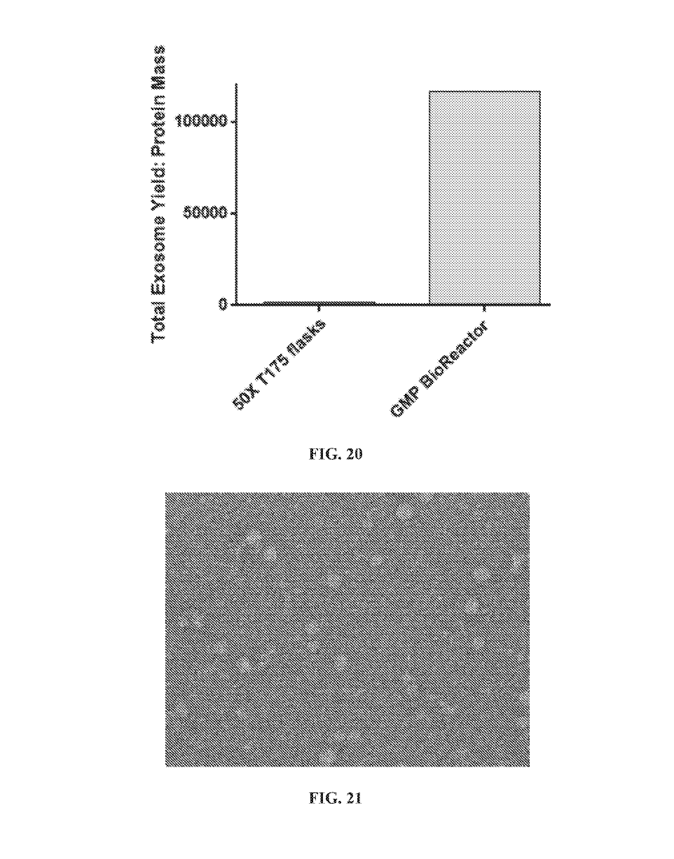

Cells cultured in a hollow fiber reactor system generate much higher yields of exosomes and/or microvesicles as compared to standard tissue culture flasks (FIG. 20). Further, use of the hollow fiber reactor system generates exosomes and/or microvesicles of canonical morphology and diameter (FIG. 21). Exosomes may be quantified using a protein concentration kit (e.g. DC assay) and/or using a NanoSight machine. Size distribution of exosomes is obtained using a NanoSight machine or other particle analyzer such as Izon or flow cytometer. Electron microscopy is used to demonstrate that the exosomes are of canonical morphology and size. Further validation may be performed with in vitro assays including a migration assay, tubule formation, and immune modulation (e.g. mixed lymphocyte reaction) prior to in vivo studies.

### Example 4

Lyopholization of Exosomes and/or Microvesicles

In some embodiments, lyophilization of exosomes and/or microvesicles of the present disclosure is practiced with use of a condenser, a vacuum pump, and a freeze-dryer. In the above methods, the manifold is assembled to ensure that a good vacuum (100 μbar or less) is achieved. The condenser should be set to −50° C. or lower. Concentrated exosome and/or microvesicle solution is dispensed into microcentrifuge tubes or other suitable containers appropriate for the scale of the condense, vacuum pump, and/or freeze dryer used. The tubes should not be more than 33% full. The lid of the tubes is pierced with a hole or removed and replaced with Parafilm or other covering pierced with several holes. The microcentrifuge tubes are snap frozen by any method well known in the art, e.g. dipping until partially submersed in liquid nitrogen or dry/acetone or alternatively freezing in a suitable spark-proof deep freezer set to negative 40° C. or lower. Once frozen, tubes are placed into a Quickfit style round-bottom flask or other suitable container for the size of tubes used. The outside of glass is cooled to −60° C. or below and attached to the manifold. The vacuum is applied and checked to ensure that it achieved returns to below 100 μbar. Samples are then allowed to completely warm to room temperature overnight (approximately 16 hours) or less for volatile solvents. Following this warming, the vacuum is released by switching the manifold valve slowly to prevent material ablating from the tubes. In some embodiments, the system is left on and fractions are dried over several days before the condenser is thawed out. In some embodiments, multiple flasks on a manifold are used and different flasks are removed at different times depending on when they have completed drying.

### Example 5

### Stroke

To establish a rat model of stroke with middle cerebral artery occlusion (MCAO), rats are first anesthetized using inhaled isofurane (3% for induction followed by 2% for maintenance). Fur on the incision site is removed using Nair and skin is cleaned and sterilized sequentially with sterile PBS, 75% ethanol and betadine. A midline neck incision is made and the soft tissues are pulled apart. The left common carotid artery (LCCA) is carefully dissected free from the surrounding nerves (without harming the vagal nerve) and a ligature is made using 6.0/7.0 suture. 5.0 suture can also be used. The left external carotid artery (LECA) is then separated and a second knot is made. Next, the left internal carotid artery (LICA) is isolated and a knot is prepared with a 6.0 filament. After obtaining a good view of the left internal carotid artery (LICA) and the left pterygopalatine artery (LPA), both arteries are clipped, using a microvascular clip. A small hole is cut in the LCCA before it bifurcates to the LECA and the LICA. A monofilament made of 8.0 nylon coated with silicon hardener mixture is then introduced into the LICA, until it stops at the clip. Attention has to be paid not to enter the occipital artery. The clipped arteries are opened while the filament is inserted into the LICA to occlude the origin of the LMCA in the circle of Willis. The third knot on the LICA is closed to fix the filament in position.

Using the above MCAO model, applicants demonstrated the therapeutic effects of exosomes in a rat model of stroke. To test whether exosomes are taken up by relevant target cell populations, MSC-Stroke exosomes are prepared by exposing MSCs to conditions that mimic the microenvironment experienced by MSC's upon injection into tissues affected by ischemia-related diseases (hypoxia, serum deprivation). Human bone marrow aspirates from young adult, non-smoking males were obtain from Lonza (Allendale, N.J.). For MSC isolation and expansion, bone marrow aspirates were passed through 90 μm pore strainers for isolation of bone spicules. Then, the strained bone marrow aspirates were diluted with equal volume of phosphate-buffered saline (PBS) and centrifuged over Ficoll (GE Healthcare, Waukesha, Wis.) for 30 minutes at 700 g. Next, mononuclear cells and bone spicules were plated in plastic culture flasks, using minimum essential media α (MEM-α) (HyClone Thermo Scientific, Waltham, Mass.) supplemented with 10% fetal bovine serum (FBS; Atlanta Biologicals, Lawrenceville, Ga.) that had been screened for optimal MSC growth. After 2 days, nonadherent cells were removed by 2-3 washing steps with PBS. After passage 2 MSCs were expanded in 20% FBS and MSCs from passages 5-6 were used for experimentation. For serum starvation, MSCs were washed 3 times with PBS and cultured in exosome isolation media consisting of OptiMEM without phenol red with 1% L-Glut (IC) (Life Technologies, Carlsbad, Calif.) for 40 hours. For serum starvation plus low oxygen conditions (PAD) MSC were cultured in exosome isolation media under 1% oxygen tension for 40 hours. Pooled human HUVECS were purchased from Lonza (Allendale, N.J.) and cultured according to manufacturers instructions using EndoGRO-LS Complete media from Millipore (Billerica, Mass.).

MSCs were washed 3 times with PBS and switched to exosome isolation media; either 20% FBS media that was pre-cleared of exosomes via 18 hour 120,000×g centrifugation, or OptiMEM (Life Technologies, Carlsbad, Calif.) and were conditioned for 40 hours prior to vesicle isolation. Microvesicles (MV) were isolated as described herein. Briefly conditioned media was cleared of cells and cell debris via centrifugation (500×g and 1000×g respectively), then spun at 17,000×g pellet to isolate MVs. Exosomes were isolated as described herein. Briefly, for proteomics studies exosomes were isolated using 0.22 μm filtration to get rid of cells, cell debris and microvesicles prior to being spun at 120,000×g for 2 hours, the pellet was then washed with 39 mLs of PBS and spun again at 120,000×g for 2 hours. All ultracentrifuge steps were performed with a Ti70 rotor in polyallomer quick seal tubes (Beckman Coulter, Brea, Calif.). Vesicle concentration was determined using DC assay (BioRad, Hercules, Calif.) and size distribution assessed using NanoSight LM10HS (Malvern, Amesbury, Mass.).

To assess the ability of MSC exosomes to influence a target cell population, exosomes were labeled with a fluorescent label and exposed to human primary endothelial cells. Uptake of exosomes can be observed after 1 hour using fluorescence microscopy. This result demonstrates that exosomes are absorbed by cells that are therapeutic targets for human treatment of ischemic stroke. Further, exposure of target cell populations (e.g. endothelial cells) to MSC-Stroke exosomes induces migration within 6 hours and tubule formation within 15 hours, demonstrating that exosomes are capable of inducing an angiogenic effect, an important feature of a potential therapeutic for stroke.

Exosome treatment is capable of inducing therapeutic responses in the MCAO model. MSC-stroke derived exosomes (100 ug/mL) can be injected intracranially, intra-arterially, or intravenously into MCAO rats. Treatment with exosomes improved rat performance in a cylinder test of asymmetric paw usage and resulted in a reduction of the inflammatory cytokine IL-1β in area surrounding the stroke infarct. This data indicates the robustness and reproducibility of the exosomes' ability produce stroke-relevant therapeutic effects (e.g. functional recovery via the motor skills assay and reduction in inflammation) by multiple routes of delivery.

### EQUIVALENTS

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

SEQUENCE LISTING

miR-150
SEQ ID NO: 1

| | |
|---|---|
| 1 | ctccccatgg ccctgtctcc caacccttgt accagtgctg ggctcagacc ctggtacagg |
| 61 | cctgggggac agggacctgg ggac |

miR-126
SEQ ID NO: 2

| | |
|---|---|
| 1 | cgctggcgac gggacattat tacttttggt acgcgctgtg acacttcaaa ctcgtaccgt |
| 61 | gagtaataat gcgccgtcca cggca |

miR-296
SEQ ID NO: 3

| | |
|---|---|
| 1 | aggacccttc cagagggccc cccctcaatc ctgttgtgcc taattcagag ggttgggtgg |
| 61 | aggctctcct gaagggctct |

let-7
SEQ ID NO: 4

| | |
|---|---|
| 1 | tgggatgagg tagtaggttg tatagtttta gggtcacacc caccactggg agataactat |
| 61 | acaatctact gtctttccta |

PDGFR-A
SEQ ID NO: 5

| | |
|---|---|
| 1 | aagagcaaaa agcgaaggcg caatctggac actgggagat tcggagcgca gggagtttga |
| 61 | gagaaacttt tattttgaag agaccaaggt tgaggggggg cttatttcct gacagctatt |
| 121 | tacttagagc aaatgattag ttttagaagg atggactata acattgaatc aattacaaaa |
| 181 | cgcggttttt gagcccatta ctgttggagc tacagggaga gaaacagagg aggagactgc |
| 241 | aagagatcat tggaggccgt gggcacgctc tttactccat gtgtgggaca ttcattgcgg |
| 301 | aataacatcg gaggagaagt ttcccagagc tatggggact tcccatccgg cgttcctggt |
| 361 | cttaggctgt cttctcacag ggctgagcct aatcctctgc cagctttcat taccctctat |
| 421 | ccttccaaat gaaaatgaaa aggttgtgca gctgaattca tccttttctc tgagatgctt |
| 481 | tggggagagt gaagtgagct ggcagtaccc catgtctgaa gaagagagct ccgatgtgga |
| 541 | aatcagaaat gaagaaaaca acagcggcct ttttgtgacg gtcttggaag tgagcagtgc |
| 601 | ctcggcggcc cacacagggt tgtacacttg ctattacaac cacactcaga cagaagagaa |
| 661 | tgagcttgaa ggcaggcaca tttacatcta tgtgccagac ccagatgtag cctttgtacc |
| 721 | tctaggaatg acggattatt tagtcatcgt ggaggatgat gattctgcca ttataccttg |
| 781 | tcgcacaact gatcccgaga ctcctgtaac cttacacaac agtgaggggg tggtacctgc |
| 841 | ctcctacgac agcagacagg gctttaatgg gaccttcact gtagggccct atatctgtga |
| 901 | ggccaccgtc aaaggaaaga agttccagac catcccattt aatgtttatg ctttaaaagc |
| 961 | aacatcagag ctggatctag aaatggaagc tcttaaaacc gtgtataagt caggggaaac |
| 1021 | gattgtggtc acctgtgctg tttttaacaa tgaggtggtt gaccttcaat ggacttaccc |
| 1081 | tggagaagtg aaaggcaaag gcatcacaat gctggaagaa atcaaagtcc catccatcaa |
| 1141 | attggtgtac actttgacgg tccccgaggc cacggtgaaa gacagtggag attacgaatg |
| 1201 | tgctgcccgc caggctacca gggaggtcaa agaaatgaag aaagtcacta tttctgtcca |
| 1261 | tgagaaaggt ttcattgaaa tcaaacccac cttcagccag ttggaagctg tcaacctgca |
| 1321 | tgaagtcaaa cattttgttg tagaggtgcg ggcctaccca cctcccagga tatcctggct |
| 1381 | gaaaaacaat ctgactctga ttgaaaatct cactgagatc accactgatg tggaaaagat |
| 1441 | tcaggaaata aggtatcgaa gcaaattaaa gctgatccgt gctaaggaag aagacagtgg |
| 1501 | ccattatact attgtagctc aaaatgaaga tgctgtgaag agctatactt ttgaactgtt |
| 1561 | aactcaagtt ccttcatcca ttctggactt ggtcgatgat caccatggct caactggggg |
| 1621 | acagacggtg aggtgcacag ctgaaggcac gccgcttcct gatattgagt ggatgatatg |
| 1681 | caaagatatt aagaaatgta ataatgaaac ttcctggact attttggcca acaatgtctc |
| 1741 | aaacatcatc acggagatcc actcccgaga caggagtacc gtggagggcc gtgtgacttt |
| 1801 | cgccaaagtg gaggagacca tcgccgtgcg atgcctggct aagaatctcc ttggagctga |
| 1861 | gaaccgagag ctgaagctgg tggctcccac cctgcgttct gaactcacgg tggctgctgc |
| 1921 | agtcctggtg ctgttggtga ttgtgatcat ctcacttatt gtcctggttg tcatttggaa |
| 1981 | acagaaaccg aggtatgaaa ttcgctggag ggtcattgaa tcaatcagcc cagatggaca |
| 2041 | tgaatatatt tatgtggacc cgatgcagct gccttatgac tcaagatggg agtttccaag |
| 2101 | agatggacta gtgcttggtc gggtcttggg gtctggagcg tttgggaagg tggttgaagg |
| 2161 | aacagcctat ggattaagcc ggtcccaacc tgtcatgaaa gttgcagtga agatgctaaa |
| 2221 | acccacggcc agatccagtg aaaaacaagc tctcatgtct gaactgaaga taatgactca |
| 2281 | cctggggcca catttgaaca ttgtaaactt gctgggagcc tgcaccaagt caggccccat |
| 2341 | ttacatcatc acagagtatt gcttctatgg agatttggtc aactatttgc ataagaatag |
| 2401 | ggatagcttc ctgagccacc acccagagaa gccaaagaaa gagctggata tctttggatt |
| 2461 | gaaccctgct gatgaaagca cacggagcta tgttatttta tcttttgaaa acaatggtga |
| 2521 | ctacatggac atgaagcagg ctgatactac acagtatgtc cccatgctag aaaggaaaga |
| 2581 | ggtttctaaa tattccgaca tccagagatc actctatgat cgtccagcct catataagaa |
| 2641 | gaaatctatg ttagactcag aagtcaaaaa cctcctttca gatgataact cagaaggcct |
| 2701 | tactttattg gatttgttga gcttcaccta tcaagttgcc cgaggaatgg agtttttggc |
| 2761 | ttcaaaaaat tgtgtccacc gtgatctggc tgctcgcaac gtcctcctgg cacaaggaaa |
| 2821 | aattgtgaag atctgtgact ttggcctggc cagagacatc atgcatgatt cgaactatgt |
| 2881 | gtcgaaaggc agtacctttc tgcccgtgaa gtggatggct cctgagagca tctttgacaa |
| 2941 | cctctacacc acactgagtg atgtctggtc ttatggcatt ctgctctggg agatcttttc |
| 3001 | ccttggtggc accccttacc ccggcatgat ggtggattct actttctaca ataagatcaa |
| 3061 | gagtgggtac cggatggcca agcctgacca cgctaccagt gaagtctacg agatcatggt |
| 3121 | gaaatgctgg aacagtgagc cggagaagag accctccttt taccacctga gtgagattgt |
| 3181 | ggagaatctg ctgcctggac aatataaaaa gagttatgaa aaaattcacc tggacttcct |
| 3241 | gaagagtgac catcctgctg tggcacgcat gcgtgtggac tcagacaatg catacattgg |
| 3301 | tgtcacctac aaaaacgagg aagacaagct gaaggactgg gagggtggtc tggatgagca |
| 3361 | gagactgagc gctgacagtg gctacatcat tcctctgcct gacattgacc ctgtccctga |
| 3421 | ggaggaggac ctgggcaaga ggaacagaca cagctcgcag acctctgaag agagtgccat |
| 3481 | tgagacgggt tccagcagtt ccaccttcat caagagagag gacgagacca ttgaagacat |
| 3541 | cgacatgatg gatgacatcg gcatagactc ttcagacctg gtggaagaca gcttcctgta |
| 3601 | actggcggat tcgaggggtt ccttccactt ctggggccac ctctggatcc cgttcagaaa |
| 3661 | accactttat tgcaatgcag aggttgagag gaggacttgg ttgatgttta aagagaagtt |
| 3721 | cccagccaag ggcctcgggg agcgttctaa atatgaatga atgggatatt ttgaaatgaa |
| 3781 | ctttgtcagt gttgcctctt gcaatgcctc agtagcatct cagtggtgtg tgaagtttgg |
| 3841 | agatagatgg ataagggaat aataggccac agaaggtgaa ctttgtgctt caaggacatt |
| 3901 | ggtgagagtc caacagacac aatttatact gcgacagaac ttcagcattg taattatgta |
| 3961 | aataactcta accaaggctg tgtttagatt gtattaacta tcttctttgg acttctgaag |
| 4021 | agaccactca atccatccat gtacttccct cttgaaacct gatgtcagct gctgttgaac |
| 4081 | tttttaaaga agtgcatgaa aaaccatttt tgaaccttaa aaggtactgg tactatagca |
| 4141 | ttttgctatc ttttttagtg ttaaagagat aaagaataat aattaaccaa ccttgtttaa |
| 4201 | tagatttggg tcatttagaa gcctgacaac tcattttcat attgtaatct atgtttataa |
| 4261 | tactactact gttatcagta atgctaaatg tgtaataatg taacatgatt tccctccaga |
| 4321 | gaaagcacaa tttaaaacaa tccttactaa gtaggtgatg agtttgacag tttttgacat |
| 4381 | ttatattaaa taacatgttt ctctataaag tatggtaata gctttagtga attaaattta |
| 4441 | gttgagcata gagaacaaag taaaagtagt gttgtccagg aagtcagaat ttttaactgt |
| 4501 | actgaatagg ttccccaatc catcgtatta aaaaacaatt aactgccctc tgaaataatg |
| 4561 | ggattagaaa caaacaaaac tcttaagtcc taaaagttct caatgtagag gcataaacct |
| 4621 | gtgctgaaca taacttctca tgtatattac ccaatggaaa atataatgat cagcaaaaag |
| 4681 | actggatttg cagaagtttt tttttttttt ttcttcatgc ctgatgaaag ctttggcgac |
| 4741 | cccaatatat gtattttttg aatctatgaa cctgaaaagg gtcagaagga tgcccagaca |
| 4801 | tcagcctcct tctttcaccc cttaccccaa agagaaagag tttgaaactc gagaccataa |
| 4861 | agatattctt tagtggaggc tggatgtgca ttagcctgga tcctcagttc tcaaatgtgt |
| 4921 | gtggcagcca ggatgactag atcctgggtt tccatccttg agattctgaa gtatgaagtc |
| 4981 | tgagggaaac cagagtctgt atttttctaa actccctggc tgttctgatc ggccagtttt |
| 5041 | cggaaacact gacttaggtt tcaggaagtt gccatgggaa acaaataatt tgaactttgg |
| 5101 | aacagggttg gcattcaacc acgcaggaag cctactattt aaatccttgg cttcaggtta |
| 5161 | gtgacattta atgccatcta gctagcaatt gcgaccttaa tttaactttc cagtcttagc |
| 5221 | tgaggctgag aaagctaaag tttggttttg acaggttttc caaaagtaaa gatgctactt |
| 5281 | cccactgtat gggggagatt gaactttccc cgtctcccgt cttctgcctc ccactccata |
| 5341 | ccccgccaag gaaaggcatg tacaaaaatt atgcaattca gtgttccaag tctctgtgta |
| 5401 | accagctcag tgttttggtg gaaaaaacat tttaagtttt actgataatt tgaggttaga |
| 5461 | tgggaggatg aattgtcaca tctatccaca ctgtcaaaca ggttggtgtg ggttcattgg |
| 5521 | cattctttgc aatactgctt aattgctgat accatatgaa tgaaacatgg gctgtgatta |
| 5581 | ctgcaatcac tgtgctatcg gcagatgatg ctttggaaga tgcagaagca ataataaagt |
| 5641 | acttgactac ctactggtgt aatctcaatg caagccccaa ctttcttatc caactttttc |
| 5701 | atagtaagtg cgaagactga gccagattgg ccaattaaaa acgaaaacct gactaggttc |
| 5761 | tgtagagcca attagacttg aaatacgttt gtgtttctag aatcacagct caagcattct |
| 5821 | gtttatcgct cactctccct tgtacagcct tattttgttg gtgctttgca ttttgatatt |
| 5881 | gctgtgagcc ttgcatgaca tcatgaggcc ggatgaaact tctcagtcca gcagtttcca |
| 5941 | gtcctaacaa atgctcccac ctgaatttgt atatgactgc atttgtgtgt gtgtgtgtgt |
| 6001 | tttcagcaaa ttccagattt gtttcctttt ggcctcctgc aaagtctcca gaagaaaatt |
| 6061 | tgccaatctt tcctactttc tatttttatg atgacaatca aagccggcct gagaaacact |
| 6121 | atttgtgact ttttaaacga ttagtgatgt ccttaaaatg tggtctgcca atctgtacaa |
| 6181 | aatggtccta tttttgtgaa gagggacata agataaaatg atgttataca tcaatatgta |
| 6241 | tatatgtatt tctatataga cttggagaat actgccaaaa catttatgac aagctgtatc |
| 6301 | actgccttcg tttatatttt tttaactgtg ataatcccca caggcacatt aactgttgca |
| 6361 | cttttgaatg tccaaaattt atattttaga aataataaaa agaaagatac ttacatgttc |
| 6421 | ccaaaacaat ggtgtggtga atgtgtgaga aaaactaact tgatagggtc taccaataca |
| 6481 | aaatgtatta cgaatgcccc tgttcatgtt tttgttttaa aacgtgtaaa tgaagatctt |
| 6541 | tatatttcaa taaatgatat ataatttaaa gtta |

PDGFR-B
SEQ ID NO: 6

| | |
|---|---|
| 1 | ctcctgaggc tgccagcagc cagcagtgac tgcccgccct atctgggacc caggatcgct |
| 61 | ctgtgagcaa cttggagcca gagaggagat caacaaggag gaggagagag ccggcccctc |
| 121 | agccctgctg cccagcagca gcctgtgctc gccctgccca acgcagacag ccagacccag |
| 181 | ggcggcccct ctggcggctc tgctcctccc gaaggatgct tggggagtga ggcgaagctg |
| 241 | ggccgctcct ctcccctaca gcagccccct tcctccatcc ctctgttctc ctgagccttc |
| 301 | aggagcctgc accagtcctg cctgtccttc tactcagctg ttacccactc tgggaccagc |
| 361 | agtctttctg ataactggga gagggcagta aggaggactt cctggagggg gtgactgtcc |
| 421 | agagcctgga actgtgccca caccagaagc catcagcagc aaggacacca tgcggcttcc |
| 481 | gggtgcgatg ccagctctgg ccctcaaagg cgagctgctg ttgctgtctc tcctgttact |
| 541 | tctggaacca cagatctctc agggcctggt cgtcacaccc ccggggccag agcttgtcct |
| 601 | caatgtctcc agcaccttcg ttctgacctg ctcgggttca gctccggtgg tgtgggaacg |
| 661 | gatgtcccag gagcccccac aggaaatggc caaggcccag gatggcacct tctccagcgt |
| 721 | gctcacactg accaacctca ctgggctaga cacgggagaa tacttttgca cccacaatga |
| 781 | ctcccgtgga ctggagaccg atgagcggaa acggctctac atctttgtgc cagatcccac |
| 841 | cgtgggcttc ctccctaatg atgccgagga actattcatc tttctcacgg aaataactga |
| 901 | gatcaccatt ccatgccgag taacagaccc acagctggtg gtgacactgc acgagaagaa |
| 961 | aggggacgtt gcactgcctg tcccctatga tcaccaacgt ggcttttctg gtatctttga |
| 1021 | ggacagaagc tacatctgca aaaccaccat tggggacagg gaggtggatt ctgatgccta |
| 1081 | ctatgtctac agactccagg tgtcatccat caacgtctct gtgaacgcag tgcagactgt |
| 1141 | ggtccgccag ggtgagaaca tcaccctcat gtgcattgtg atcgggaatg aggtggtcaa |
| 1201 | cttcgagtgg acataccccc gcaaagaaag tgggcggctg gtggagccgg tgactgactt |
| 1261 | cctcttggat atgccttacc acatccgctc catcctgcac atccccagtg ccgagttaga |
| 1321 | agactcgggg acctacacct gcaatgtgac ggagagtgtg aatgaccatc aggatgaaaa |
| 1381 | ggccatcaac atcaccgtgg ttgagagcgg ctacgtgcgg ctcctgggag aggtgggcac |
| 1441 | actacaattt gctgagctgc atcggagccg gacactgcag gtagtgttcg aggcctaccc |
| 1501 | accgcccact gtcctgtggt tcaaagacaa ccgcaccctg ggcgactcca gcgctggcga |
| 1561 | aatcgccctg tccacgcgca acgtgtcgga gacccggtat gtgtcagagc tgacactggt |
| 1621 | tcgcgtgaag gtggcagagg ctggccacta caccatgcgg gccttccatg aggatgctga |
| 1681 | ggtccagctc tccttccagc tacagatcaa tgtccctgtc cgagtgctgg agctaagtga |
| 1741 | gagccaccct gacagtgggg aacagacagt ccgctgtcgt ggccggggca tgccccagcc |
| 1801 | gaacatcatc tggtctgcct gcagagacct caaaaggtgt ccacgtgagc tgccgcccac |
| 1861 | gctgctgggg aacagttccg aagaggagag ccagctggag actaacgtga cgtactggga |
| 1921 | ggaggagcag gagtttgagg tggtgagcac actgcgtctg cagcacgtgg atcggccact |
| 1981 | gtcggtgcgc tgcacgctgc gcaacgctgt gggccaggac acgcaggagg tcatcgtggt |
| 2041 | gccacactcc ttgcccttta aggtggtggt gatctcagcc atcctggccc tggtggtgct |
| 2101 | caccatcatc tcccttatca tcctcatcat gctttggcag aagaagccac gttacgagat |
| 2161 | ccgatggaag gtgattgagt ctgtgagctc tgacggccat gagtacatct acgtggaccc |
| 2221 | catgcagctg ccctatgact ccacgtggga gctgccgcgg gaccagcttg tgctgggacg |
| 2281 | caccctcggc tctggggcct ttgggcaggt ggtggaggcc acggctcatg gcctgagcca |
| 2341 | ttctcaggcc acgatgaaag tggccgtcaa gatgcttaaa tccacagccc gcagcagtga |
| 2401 | gaagcaagcc cttatgtcgg agctgaagat catgagtcac cttgggcccc acctgaacgt |
| 2461 | ggtcaacctg ttgggggcct gcaccaaagg aggacccatc tatatcatca ctgagtactg |
| 2521 | ccgctacgga gacctggtgg actacctgca ccgcaacaaa cacaccttcc tgcagcacca |
| 2581 | ctccgacaag cgccgcccgc ccagcgcgga gctctacagc aatgctctgc ccgttgggct |
| 2641 | ccccctgccc agccatgtgt ccttgaccgg ggagagcgac ggtggctaca tggacatgag |
| 2701 | caaggacgag tcggtggact atgtgcccat gctggacatg aaaggagacg tcaaatatgc |
| 2761 | agacatcgag tcctccaact acatggcccc ttacgataac tacgttccct ctgcccctga |
| 2821 | gaggacctgc cgagcaactt tgatcaacga gtctccagtg ctaagctaca tggacctcgt |
| 2881 | gggcttcagc taccaggtgg ccaatggcat ggagtttctg gcctccaaga actgcgtcca |
| 2941 | cagagacctg gcggctagga acgtgctcat ctgtgaaggc aagctggtca agatctgtga |
| 3001 | ctttggcctg gctcgagaca tcatgcggga ctcgaattac atctccaaag gcagcacctt |
| 3061 | tttgccttta aagtggatgg ctccggagag catcttcaac agcctctaca ccaccctgag |
| 3121 | cgacgtgtgg tccttcggga tcctgctctg ggagatcttc accttgggtg gcacccctta |
| 3181 | cccagagctg cccatgaacg agcagttcta caatgccatc aaacggggtt accgcatggc |
| 3241 | ccagcctgcc catgcctccg acgagatcta tgagatcatg cagaagtgct gggaagagaa |
| 3301 | gtttgagatt cggcccccct tctcccagct ggtgctgctt ctcgagagac tgttgggcga |
| 3361 | aggttacaaa aagaagtacc agcaggtgga tgaggagttt ctgaggagtg accacccagc |
| 3421 | catccttcgg tcccaggccc gcttgcctgg gttccatggc ctccgatctc ccctggacac |
| 3481 | cagctccgtc ctctatactg ccgtgcagcc caatgagggt gacaacgact atatcatccc |
| 3541 | cctgcctgac cccaaacccg aggttgctga cgagggccca ctggagggtt cccccagcct |
| 3601 | agccagctcc accctgaatg aagtcaacac ctcctcaacc atctcctgtg acagccccct |
| 3661 | ggagccccag gacgaaccag agccagagcc ccagcttgag ctccaggtgg agccggagcc |
| 3721 | agagctggaa cagttgccgg attcggggtg ccctgcgcct cgggcggaag cagaggatag |
| 3781 | cttcctgtag ggggctggcc cctaccctgc cctgcctgaa gctccccccc tgccagcacc |
| 3841 | cagcatctcc tggcctggcc tgaccgggct tcctgtcagc caggctgccc ttatcagctg |
| 3901 | tccccttctg gaagctttct gctcctgacg tgttgtgccc caaaccctgg ggctggctta |
| 3961 | ggaggcaaga aaactgcagg ggccgtgacc agccctctgc ctccagggag gccaactgac |
| 4021 | tctgagccag ggttccccca gggaactcag ttttcccata tgtaagatgg gaaagttagg |
| 4081 | cttgatgacc cagaatctag gattctctcc ctggctgaca ggtggggaga ccgaatccct |
| 4141 | ccctgggaag attcttggag ttactgaggt ggtaaattaa cttttttctg ttcagccagc |
| 4201 | tacccctcaa ggaatcatag ctctctcctc gcacttttat ccacccagga gctagggaag |
| 4261 | agaccctagc ctccctggct gctggctgag ctagggccta gccttgagca gtgttgcctc |
| 4321 | atccagaaga aagccagtct cctccctatg atgccagtcc ctgcgttccc tggcccgagc |
| 4381 | tggtctgggg ccattaggca gcctaattaa tgctggaggc tgagccaagt acaggacacc |
| 4441 | cccagcctgc agcccttgcc cagggcactt ggagcacacg cagccatagc aagtgcctgt |
| 4501 | gtccctgtcc ttcaggccca tcagtcctgg ggctttttct ttatcaccct cagtcttaat |
| 4561 | ccatccacca gagtctagaa ggccagacgg gccccgcatc tgtgatgaga atgtaaatgt |
| 4621 | gccagtgtgg agtggccacg tgtgtgtgcc agtatatggc cctggctctg cattggacct |
| 4681 | gctatgaggc tttggaggaa tccctcaccc tctctgggcc tcagtttccc cttcaaaaaa |
| 4741 | tgaataagtc ggacttatta actctgagtg ccttgccagc actaacattc tagagtattc |
| 4801 | caggtggttg cacatttgtc cagatgaagc aaggccatat accctaaact tccatcctgg |
| 4861 | gggtcagctg ggctcctggg agattccaga tcacacatca cactctgggg actcaggaac |
| 4921 | catgcccctt ccccaggccc ccagcaagtc tcaagaacac agctgcacag gccttgactt |
| 4981 | agagtgacag ccggtgtcct ggaaagcccc cagcagctgc cccagggaca tgggaagacc |
| 5041 | acgggacctc tttcactacc cacgatgacc tccgggggta tcctgggcaa aagggacaaa |
| 5101 | gagggcaaat gagatcacct cctgcagccc accactccag cacctgtgcc gaggtctgcg |
| 5161 | tcgaagacag aatggacagt gaggacagtt atgtcttgta aaagacaaga agcttcagat |
| 5221 | gggtacccca agaaggatgt gagaggtggg cgctttggag gtttgcccct cacccaccag |
| 5281 | ctgccccatc cctgaggcag cgctccatgg gggtatggtt ttgtcactgc ccagacctag |
| 5341 | cagtgacatc tcattgtccc cagcccagtg ggcattggag gtgccagggg agtcagggtt |
| 5401 | gtagccaaga cgcccccgca cggggagggt tgggaagggg gtgcaggaag ctcaacccct |
| 5461 | ctgggcacca accctgcatt gcaggttggc accttacttc cctgggatcc ccagagttgg |
| 5521 | tccaaggagg gagagtgggt tctcaatacg gtaccaaaga tataatcacc taggtttaca |
| 5581 | aatattttta ggactcacgt taactcacat ttatacagca gaaatgctat tttgtatgct |
| 5641 | gttaagtttt tctatctgtg tacttttttt taagggaaag attttaatat taaacctggt |
| 5701 | gcttctcact cacaaaaa |

COL6A3
SEQ ID NO: 8

| | |
|---|---|
| 1 | aagccctgac tggtatccct ggccccagtc cagtttggag ctcagtcttc caccaaaggc |
| 61 | cgttcagttc tcctgggctc cagcctcctg caaggactgc aagagttttc ctccgcagct |
| 121 | ctgagtctcc acttttttgg tggagaaagg ctgcaaaaag aaaaagagac gcagtgagtg |
| 181 | ggaaaagtat gcatcctatt caaacctaat tgaatcgagg agcccaggga cacacgcctt |
| 241 | caggtttgct caggggttca tatttggtgc ttagacaaat tcaaaatgag gaaacatcgg |
| 301 | cacttgccct tagtggccgt cttttgcctc tttctctcag gctttcctac aactcatgcc |
| 361 | cagcagcagc aagcagatgt caaaaatggt gcggctgctg atataatatt tctagtggat |
| 421 | tcctcttgga ccattggaga ggaacatttc caacttgttc gagagtttct atatgatgtt |
| 481 | gtaaaatcct tagctgtggg agaaaatgat ttccattttg ctctggtcca gttcaacgga |
| 541 | aacccacata ccgagttcct gttaaatacg tatcgtacta aacaagaagt cctttctcat |
| 601 | atttccaaca tgtcttatat tgggggaacc aatcagactg gaaaaggatt agaatacata |
| 661 | atgcaaagcc acctcaccaa ggctgctgga agccgggccg gtgacggagt ccctcaggtt |
| 721 | atcgtagtgt taactgatgg acactcgaag gatggccttg ctctgccctc agcggaactt |
| 781 | aagtctgctg atgttaacgt gtttgcaatt ggagttgagg atgcagatga aggagcgtta |
| 841 | aaagaaatag caagtgaacc gctcaatatg catatgttca acctagagaa ttttacctca |
| 901 | cttcatgaca tagtaggaaa cttagtgtcc tgtgtgcatt catccgtgag tccagaaagg |
| 961 | gctggggaca cggaaaccct taaagacatc acagcacaag actctgctga cattattttc |
| 1021 | cttattgatg gatcaaacaa caccggaagt gtcaatttcg cagtcattct cgacttcctt |
| 1081 | gtaaatctcc ttgagaaact cccaattgga actcagcaga tccgagtggg ggtggtccag |
| 1141 | tttagcgatg agcccagaac catgttctcc ttggacacct actccaccaa ggcccaggtt |
| 1201 | ctgggtgcag tgaaagccct cgggtttgct ggtggggagt tggccaatat cggcctcgcc |
| 1261 | cttgatttcg tggtggagaa ccacttcacc cgggcagggg gcagccgcgt ggaggaaggg |
| 1321 | gttccccagg tgctggtcct cataagtgcc gggccttcta gtgacgagat tcgctacggg |
| 1381 | gtggtagcac tgaagcaggc tagcgtgttc tcattcggcc ttggagccca ggccgcctcc |
| 1441 | agggcagagc ttcagcacat agctaccgat gacaacttgg tgtttactgt cccggaattc |
| 1501 | cgtagctttg gggacctcca ggagaaatta ctgccgtaca ttgttggcgt ggcccaaagg |
| 1561 | cacattgtct tgaaaccgcc aaccattgtc acacaagtca ttgaagtcaa caagagagac |
| 1621 | atagtcttcc tggtggatgg ctcatctgca ctgggactgg ccaacttcaa tgccatccga |
| 1681 | gacttcattg ctaaagtcat ccagaggctg gaaatcggac aggatcttat ccaggtggca |
| 1741 | gtggcccagt atgcagacac tgtgaggcct gaattttatt tcaataccca tccaacaaaa |
| 1801 | agggaagtca taaccgctgt gcggaaaatg aagcccctgg acggctcggc cctgtacacg |
| 1861 | ggctctgctc tagactttgt tcgtaacaac ctattcacga gttcagccgg ctaccgggct |
| 1921 | gccgagggga ttcctaagct tttggtgctg atcacaggtg gtaagtccct agatgaaatc |
| 1981 | agccagcctg cccaggagct gaagagaagc agcataatgg cctttgccat tgggaacaag |
| 2041 | ggtgccgatc aggctgagct ggaagagatc gctttcgact cctccctggt gttcatccca |
| 2101 | gctgagttcc gagccgcccc attgcaaggc atgctgcctg gcttgctggc acctctcagg |
| 2161 | accctctctg gaacccctga agttcactca aacaaaaggg atatcatctt tcttttggat |
| 2221 | ggatcagcca acgttggaaa aaccaatttc ccttatgtgc gcgactttgt aatgaaccta |
| 2281 | gttaacagcc ttgatattgg aaatgacaat attcgtgttg gtttagtgca atttagtgac |
| 2341 | actcctgtaa cggagttctc tttaaacaca taccagacca agtcagatat ccttggtcat |
| 2401 | ctgaggcagc tgcagctcca gggaggttcg ggcctgaaca caggctcagc cctaagctat |
| 2461 | gtctatgcca accacttcac ggaagctggc ggcagcagga tccgtgaaca cgtgccgcag |
| 2521 | ctcctgcttc tgctcacagc tgggcagtct gaggactcct atttgcaagc tgccaacgcc |
| 2581 | ttgacacgcg cgggcatcct gactttttgt gtgggagcta gccaggcgaa taaggcagag |
| 2641 | cttgagcaga ttgcttttaa cccaagcctg gtgtatctca tggatgattt cagctccctg |
| 2701 | ccagctttgc ctcagcagct gattcagccc ctaaccacat atgttagtgg aggtgtggag |
| 2761 | gaagtaccac tcgctcagcc agagagcaag cgagacattc tgttcctctt tgacggctca |
| 2821 | gccaatcttg tgggccagtt ccctgttgtc cgtgactttc tctacaagat tatcgatgag |
| 2881 | ctcaatgtga agccagaggg gacccgaatt gcggtggctc agtacagcga tgatgtcaag |
| 2941 | gtggagtccc gttttgatga gcaccagagt aagcctgaga tcctgaatct tgtgaagaga |
| 3001 | atgaagatca agacgggcaa agccctcaac ctgggctacg cgctggacta tgcacagagg |
| 3061 | tacatttttg tgaagtctgc tggcagccgg atcgaggatg gagtgcttca gttcctggtg |
| 3121 | ctgctggtcg caggaaggtc atctgaccgt gtggatgggc cagcaagtaa cctgaagcag |
| 3181 | agtggggttg tgcctttcat cttccaagcc aagaacgcag accctgctga gttagagcag |
| 3241 | atcgtgctgt ctccagcgtt tatcctggct gcagagtcgc ttcccaagat tggagatctt |
| 3301 | catccacaga tagtgaatct cttaaaatca gtgcacaacg gagcaccagc accagtttca |
| 3361 | ggtgaaaagg acgtggtgtt tctgcttgat ggctctgagg gcgtcaggag cggcttccct |
| 3421 | ctgttgaaag agtttgtcca gagagtggtg gaaagcctgg atgtgggcca ggaccgggtc |
| 3481 | cgcgtggccg tggtgcagta cagcgaccgg accaggcccg agttctacct gaattcatac |
| 3541 | atgaacaagc aggacgtcgt caacgctgtc cgccagctga ccctgctggg agggccgacc |
| 3601 | cccaacaccg gggccgccct ggagtttgtc ctgaggaaca tcctggtcag ctctgcggga |
| 3661 | agcaggataa cagaaggtgt gccccagctg ctgatcgtcc tcacggccga caggtctggg |
| 3721 | gatgatgtgc ggaacccctc cgtggtcgtg aagaggggtg gggctgtgcc cattggcatt |
| 3781 | ggcatcggga acgctgacat cacagagatg cagaccatct ccttcatccc ggactttgcc |
| 3841 | gtggccattc ccacctttcg ccagctgggg accgtccaac aggtcatctc tgagagggtg |
| 3901 | acccagctca cccgcgagga gctgagcagg ctgcagccgg tgttgcagcc tctaccgagc |
| 3961 | ccaggtgttg gtggcaagag ggacgtggtc tttctcatcg atgggtccca aagtgccggg |
| 4021 | cctgagttcc agtacgttcg caccctcata gagaggctgg ttgactacct ggacgtgggc |
| 4081 | tttgacacca cccgggtggc tgtcatccag ttcagcgatg accccaaggt ggagttcctg |
| 4141 | ctgaacgccc attccagcaa ggatgaagtg cagaacgcgg tgcagcggct gaggcccaag |
| 4201 | ggagggcggc agatcaacgt gggcaatgcc ctggagtacg tgtccaggaa catcttcaag |
| 4261 | aggcccctgg ggagccgcat tgaagagggc gtcccgcagt tcctggtcct catctcgtct |
| 4321 | ggaaagtctg acgatgaggt ggacgacccg gcggtggagc tcaagcagtt tggcgtggcc |
| 4381 | cctttcacga tcgccaggaa cgcagaccag gaggagctgg tgaagatctc gctgagcccc |
| 4441 | gaatatgtgt tctcggtgag caccttccgg gagctgccca gcctggagca gaaactgctg |
| 4501 | acgcccatca cgaccctgac ctcagagcag atccagaagc tcttagccag cactcgctat |
| 4561 | ccacctccag cagttgagag tgatgctgca gacattgtct ttctgatcga cagctctgag |
| 4621 | ggagttaggc cagatggctt tgcacatatt cgagattttg ttagcaggat tgttcgaaga |
| 4681 | ctcaacatcg gccccagtaa agtgagagtt ggggtcgtgc agttcagcaa tgatgtcttc |
| 4741 | ccagaattct atctgaaaac ctacagatcc caggccccgg tgctggacgc catacggcgc |
| 4801 | ctgaggctca gaggggggtc cccactgaac actggcaagg ctctcgaatt tgtggcaaga |
| 4861 | aacctctttg ttaagtctgc ggggagtcgc atagaagacg gggtgcccca acacctggtc |
| 4921 | ctggtcctgg gtggaaaatc ccaggacgat gtgtccaggt tcgcccaggt gatccgttcc |
| 4981 | tcgggcattg tgagtttagg ggtaggagac cggaacatcg acagaacaga gctgcagacc |
| 5041 | atcaccaatg accccagact ggtcttcaca gtgcgagagt tcagagagct tcccaacata |
| 5101 | gaagaaagaa tcatgaactc gtttggaccc tccgcagcca ctcctgcacc tccaggggtg |
| 5161 | gacacccctc ctccttcacg gccagagaag aagaaagcag acattgtgtt cctgttggat |
| 5221 | ggttccatca acttcaggag ggacagtttc caggaagtgc ttcgttttgt gtctgaaata |
| 5281 | gtggacacag tttatgaaga tggcgactcc atccaagtgg ggcttgtcca gtacaactct |
| 5341 | gaccccactg acgaattctt cctgaaggac ttctctacca agaggcagat tattgacgcc |
| 5401 | atcaacaaag tggtctacaa agggggaaga cacgccaaca ctaaggtggg ccttgagcac |
| 5461 | ctgcgggtaa accactttgt gcctgaggca ggcagccgcc tggaccagcg ggtccctcag |
| 5521 | attgcctttg tgatcacggg aggaaagtcg gtggaagatg cacaggatgt gagcctggcc |
| 5581 | ctcacccaga ggggggtcaa agtgtttgct gttggagtga ggaatatcga ctcggaggag |
| 5641 | gttggaaaga tagcgtccaa cagcgccaca gcgttccgcg tgggcaacgt ccaggagctg |
| 5701 | tccgaactga gcgagcaagt tttggaaact ttgcatgatg cgatgcatga aaccctttgc |
| 5761 | cctggtgtaa ctgatgctgc caaagcttgt aatctggatg tgattctggg gtttgatggt |
| 5821 | tctagagacc agaatgtttt tgtggcccag aagggcttcg agtccaaggt ggacgccatc |
| 5881 | ttgaacagaa tcagccagat gcacagggtc agctgcagcg gtggccgctc gcccaccgtg |
| 5941 | cgtgtgtcag tggtggccaa cacgccctcg ggcccggtgg aggcctttga ctttgacgag |
| 6001 | taccagccag agatgctcga gaagttccgg aacatgcgca gccagcaccc ctacgtcctc |
| 6061 | acggaggaca ccctgaaggt ctacctgaac aagttcagac agtcctcgcc ggacagcgtg |
| 6121 | aaggtggtca ttcattttac tgatggagca gacggagatc tggctgattt acacagagca |
| 6181 | tctgagaacc tccgccaaga aggagtccgt gccttgatcc tggtgggcct tgaacgagtg |
| 6241 | gtcaacttgg agcggctaat gcatctggag tttgggcgag ggtttatgta tgacaggccc |
| 6301 | ctgaggctta acttgctgga cttggattat gaactagcgg agcagcttga caacattgcc |
| 6361 | gagaaagctt gctgtggggt tccctgcaag tgctctgggc agaggggaga ccgcgggccc |
| 6421 | atcggcagca tcgggccaaa gggtattcct ggagaagacg gctaccgagg ctatcctggt |
| 6481 | gatgagggtg gacccggtga gcgtggtccg cctggtgtga acggcactca aggtttccag |
| 6541 | ggctgcccgg gccagagagg agtaaagggc tctcggggat tcccaggaga gaagggcgaa |
| 6601 | gtaggagaaa ttggactgga tggtctggat ggtgaagatg gagacaaagg attgcctggt |
| 6661 | tcttctggag agaaagggaa tcctggaaga aggggtgata aaggacctcg aggagagaaa |
| 6721 | ggagaaagag gagatgttgg gattcgaggg gacccgggta acccaggaca agacagccag |
| 6781 | gagagaggac ccaaaggaga aaccggtgac ctcggcccca tgggtgtccc agggagagat |
| 6841 | ggagtacctg gaggacctgg agaaactggg aagaatggtg gctttggccg aaggggaccc |
| 6901 | cccggagcta agggcaacaa gggcggtcct ggccagccgg gctttgaggg agagcagggg |
| 6961 | accagaggtg cacagggccc agctggtcct gctggtcctc cagggctgat aggagaacaa |
| 7021 | ggcatttctg gacctcgggg aagcggaggt gccgctggtg ctcctggaga acgaggcaga |
| 7081 | accggtccac tgggaagaaa gggtgagccc ggagagccag gaccaaaagg aggaatcggg |
| 7141 | aaccggggcc ctcgtgggga gacgggagat gacgggagag acggagttgg cagtgaagga |
| 7201 | cgcagaggca aaaaaggaga aagaggattc cctggatacc caggaccaaa gggtaaccca |
| 7261 | ggtgaacctg ggctaaatgg aacaacagga cccaaaggca tcagaggccg aaggggaaat |
| 7321 | tcgggacctc cagggatagt tggacagaag ggagaccctg gctacccagg accagctggt |
| 7381 | cccaagggca acaggggcga ctccatcgat caatgtgccc tcatccaaag catcaaagat |
| 7441 | aaatgccctt gctgttacgg gcccctggag tgccccgtct tcccaacaga actagccttt |
| 7501 | gctttagaca cctctgaggg agtcaaccaa gacactttcg gccggatgcg agatgtggtc |
| 7561 | ttgagtattg tgaatgacct gaccattgct gagagcaact gcccacgggg ggcccgggtg |
| 7621 | gctgtggtca cctacaacaa cgaggtgacc acggagatcc ggtttgctga ctccaagagg |
| 7681 | aagtcggtcc tcctggacaa gattaagaac cttcaggtgg ctctgacatc caaacagcag |
| 7741 | agtctggaga ctgccatgtc gtttgtggcc aggaacacat ttaagcgtgt gaggaacgga |
| 7801 | ttcctaatga ggaaagtggc tgttttcttc agcaacacac ccacaagagc atccccacag |
| 7861 | ctcagagagg ctgtgctcaa gctctcagat gcggggatca cccccttgtt ccttacaagg |
| 7921 | caggaagacc ggcagctcat caacgctttg cagatcaata acacagcagt ggggcatgcg |
| 7981 | cttgtcctgc ctgcagggag agacctcaca gacttcctgg agaatgtcct cacgtgtcat |
| 8041 | gtttgcttgg acatctgcaa catcgaccca tcctgtggat ttggcagttg gaggccttcc |
| 8101 | ttcagggaca ggagagcggc agggagcgat gtggacatcg acatggcttt catcttagac |
| 8161 | agcgctgaga ccaccaccct gttccagttc aatgagatga agaagtacat agcgtacctg |
| 8221 | gtcagacaac tggacatgag cccagatccc aaggcctccc agcacttcgc cagagtggca |
| 8281 | gttgtgcagc acgcgccctc tgagtccgtg gacaatgcca gcatgccacc tgtgaaggtg |
| 8341 | gaattctccc tgactgacta tggctccaag gagaagctgg tggacttcct cagcagggga |
| 8401 | atgacacagt tgcagggaac cagggcctta ggcagtgcca ttgaatacac catagagaat |
| 8461 | gtctttgaaa gtgccccaaa cccacgggac ctgaaaattg tggtcctgat gctgacgggc |
| 8521 | gaggtgccgg agcagcagct ggaggaggcc cagagagtca tcctgcaggc caaatgcaag |
| 8581 | ggctacttct tcgtggtcct gggcattggc aggaaggtga acatcaagga ggtatacacc |
| 8641 | ttcgccagtg agccaaacga cgtcttcttc aaattagtgg acaagtccac cgagctcaac |
| 8701 | gaggagcctt tgatgcgctt cgggaggctg ttgccatcct tcgtcagcag tgaaaatgct |
| 8761 | ttttacttgt ccccagatat caggaaacag tgtgattggt tccaagggga ccaacccaca |
| 8821 | aagaaccttg tgaagtttgg tcacaaacaa gtaaatgttc cgaataacgt tacttcaagt |
| 8881 | cctacatcca acccagtgac gacaacgaag ccggtgacta cgacgaagcc ggtgaccacc |
| 8941 | acaacaaagc ctgtaaccac cacaacaaag cctgtgacta ttataaatca gccatctgtg |
| 9001 | aagccagccg ctgcaaagcc ggcccctgcg aaacctgtgg ctgccaagcc tgtggccaca |
| 9061 | aagatggcca ctgttagacc cccagtggcg gtgaagccag caacggcagc gaagcctgta |
| 9121 | gcagcaaagc cagcagctgt aagacccccc gctgctgctg ctgcaaaacc agtggcgacc |
| 9181 | aagcctgagg tccctaggcc acaggcagcc aaaccagctg ccaccaagcc agccaccact |
| 9241 | aagcccatgg ttaagatgtc ccgtgaagtc caggtgtttg agataacaga gaacagcgcc |
| 9301 | aaactccact gggagagggc tgagcccccc ggtccttatt tttatgacct caccgtcacc |
| 9361 | tcagcccatg atcagtccct ggttctgaag cagaacctca cggtcacgga ccgcgtcatt |
| 9421 | ggaggcctgc tcgctgggca gacataccat gtggctgtgg tctgctacct gaggtctcag |
| 9481 | gtcagagcca cctaccacgg aagtttcagt acaaagaaat ctcagccccc acctccacag |
| 9541 | ccagcaaggt cagcttctag ttcaaccatc aatctaatgg tgagcacaga accattggct |
| 9601 | ctcactgaaa cagatatatg caagttgccg aaagacgaag gaacttgcag ggatttcata |
| 9661 | ttaaaatggt actatgatcc aaacaccaaa agctgtgcaa gattctggta tggaggttgt |
| 9721 | ggtggaaacg aaaacaaatt tggatcacag aaagaatgtg aaaaggtttg cgctcctgtg |
| 9781 | ctcgccaaac ccggagtcat cagtgtgatg ggaacctaag cgtgggtggc caacatcata |
| 9841 | tacctcttga agaagaagga gtcagccatc gccaacttgt ctctgtagaa gctccgggtg |
| 9901 | tagattccct tgcactgtat catttcatgc tttgatttac actcgaactc gggagggaac |
| 9961 | atcctgctgc atgacctatc agtatggtgc taatgtgtct gtggaccctc gctctctgtc |
| 10021 | tccaggcagt tctctcgaat actttgaatg ttgtgtaaca gttagccact gctggtgttt |
| 10081 | atgtgaacat tcctatcaat ccaaattccc tctggagttt catgttatgc ctgttgcagg |
| 10141 | caaatgtaaa gtctagaaaa taatgcaaat gtcacggcta ctctatatac ttttgcttgg |
| 10201 | ttcatttttt ttccctttta gttaagcatg actttagatg ggaagcctgt gtatcgtgga |
| 10261 | gaaacaagag accaactttt tcattccctg cccccaattt cccagactag atttcaagct |
| 10321 | aattttcttt ttctgaagcc tctaacaaat gatctagttc agaaggaagc aaaatccctt |
| 10381 | aatctatgtg caccgttggg accaatgcct taattaaaga atttaaaaaa gttgtaatag |
| 10441 | agaatatttt tggcattcct ctaatgttgt gtgttttttt tttgtgtgtg ctggagggag |
| 10501 | gggatttaat tttaatttta aaatgtttag gaaatttata caaagaaact ttttaataaa |
| 10561 | gtatattgaa agtttcctgg gaaaaaaaaa aaaaaaaaa |

EDIL3
SEQ ID NO: 9

| | |
|---|---|
| 1 | ctctgtttgt acacagtgcg ctcccggcgg cccgctcgct cccctccagc tcacgcttca |
| 61 | ttgttctcca agtcagaagc cccgcagccg ccgcgcggag aacagcgaca gccgagcgcc |
| 121 | cggtccgcct gtctgccggt gggtctgcct gcccgcgcag cagacccggg gcggccgcgg |
| 181 | gagcccgcgc cccgcccgcc gcgcctctgc cgggacccac ccgcagcgga gggctgagcc |
| 241 | cgccggcggc tccccggagc tcacccacct ccgcgcgccg gagcgcaggc aaaaggggag |
| 301 | gaaaggctcc tctctttagt caccactctc gccctctcca agaatttgtt taacaaagcg |
| 361 | ctgaggaaag agaacgtctt cttgaattct ttagtagggg cggagtctgc tgctgccctg |
| 421 | cgctgccacc tcggctacac tgccctccgc gacgacccct gaccagccgg ggtcacgtcc |
| 481 | gggagacggg atcatgaagc gctcggtagc cgtctggctc ttggtcgggc tcagcctcgg |
| 541 | tgtcccccag ttcggcaaag gtgatatttg tgatcccaat ccatgtgaaa atggaggtat |
| 601 | ctgtttgcca ggattggctg atggttcctt ttcctgtgag tgtccagatg gcttcacaga |
| 661 | ccccaactgt tctagtgttg tggaggttgc atcagatgaa gaagaaccaa cttcagcagg |
| 721 | tccctgcact cctaatccat gccataatgg aggaacctgt gaaataagtg aagcataccg |
| 781 | aggggataca ttcataggct atgtttgtaa atgtccccga ggatttaatg ggattcactg |
| 841 | tcagcacaac ataaatgaat gcgaagttga gccttgcaaa aatggtggaa tatgtacaga |
| 901 | tcttgttgct aactattcct gtgagtgccc aggcgaattt atgggaagaa attgtcaata |
| 961 | caaatgctca ggcccactgg gaattgaagg tggaattata tcaaaccagc aaatcacagc |
| 1021 | ttcctctact caccgagctc tttttggact ccaaaaatgg tatccctact atgcacgtct |
| 1081 | taataagaag gggcttataa atgcgtggac agctgcagaa aatgacagat ggccgtggat |
| 1141 | tcagataaat ttgcaaagga aaatgagagt tactggtgtg attacccaag gagccaagag |
| 1201 | gattggaagc ccagagtata taaaatccta caaaattgcc tacagtaatg atggaaagac |
| 1261 | ttgggcaatg tacaaagtga aaggcaccaa tgaagacatg gtgtttcgtg gaaacattga |
| 1321 | taacaacact ccatatgcta actctttcac accccccata aaagctcagt atgtaagact |
| 1381 | ctatccccaa gtttgtcgaa gacattgcac tttgcgaatg gaacttcttg gctgtgaact |
| 1441 | gtcgggttgt tctgagcctc tgggtatgaa atcaggacat atacaagact atcagatcac |
| 1501 | tgcctccagc atcttcagaa cgctcaacat ggacatgttc acttgggaac caaggaaagc |
| 1561 | tcggctggac aagcaaggca aagtgaatgc ctggacctct ggccacaatg accagtcaca |
| 1621 | atggttacag gtggatcttc ttgttccaac caaagtgact ggcatcatta cacaaggagc |
| 1681 | taaagatttt ggtcatgtac agtttgttgg ctcctacaaa ctggcttaca gcaatgatgg |
| 1741 | agaacactgg actgtatacc aggatgaaaa gcaaagaaaa gataaggttt tccagggaaa |
| 1801 | ttttgacaat gacactcaca gaaaaaatgt catcgaccct cccatctatg cacgacacat |
| 1861 | aagaatcctt ccttggtcct ggtacgggag gatcacattg cggtcagagc tgctgggctg |
| 1921 | cacagaggag gaatgagggg aggctacatt tcacaaccct cttccctatt tccctaaaag |
| 1981 | tatctccatg gaatgaactg tgcaaaatct gtaggaaact gaatggtttt tttttttttt |
| 2041 | tcatgaaaaa gtgctcaaat tatggtaggc aactaacggt gtttttaagg gggtctaagc |
| 2101 | ctgccttttc aatgatttaa tttgatttta ttttatccgt caaatctctt aagtaacaac |
| 2161 | acattaagtg tgaattactt ttctctcatt gtttcctgaa ttattcgcat tggtagaaat |
| 2221 | atattaggga aagaaagtag ccttcttttt atagcaagag taaaaaagtc tcaaagtcat |
| 2281 | caaataagag caagagttga tagagctttt acaatcaata ctcacctaat tctgataaaa |
| 2341 | ggaatactgc aatgttagca ataagttttt ttcttctgta atgactctac gttatcctgt |
| 2401 | ttccctgtgc ctaccaaaca ctgtcaatgt ttattacaaa attttaaaga agaatatgta |
| 2461 | acatgcagta ctgatattat aattctcatt ttactttcat tatttctaat aagagattat |
| 2521 | gtgacttctt tttcttttag ttctattcta cattcttaat attgtatatt acctgaataa |
| 2581 | ttcaattttt ttctaattga atttcctatt agttgactaa aagaagtgtc atgtttactc |
| 2641 | atatatgtag aacatgactg cctatcagta gattgatctg tatttaatat tcgttaatta |
| 2701 | aatctgcagt tttatttttg aaggaagcca taactattta atttccaaat aattgcttca |
| 2761 | taaagaatcc catactctca gtttgcacaa aagaacaaaa aatatatatg tctctttaaa |
| 2821 | tttaaatctt catttagatg gtaattacat atccttatat ttactttaaa aaatcggctt |
| 2881 | atttgtttat tttataaaaa atttagcaaa gaaatattaa tatagtgctg catagtttgg |
| 2941 | ccaagcatac tcatcatttc tttgttcagc tccacatttc ctgtgaaact aacatcttat |
| 3001 | tgagatttga aactggtggt agtttcccag gaaggcacag gtggagttat ttgtgagaag |
| 3061 | caaagtgttt actaatgaca aagtagtaaa ccattttcaa gatgaaaact gatttctatt |
| 3121 | tattttgctt caaaggtcct gaaaaaataa gcaattatca taacaatttg ttattgatac |
| 3181 | tggaggtttc attgacatgt ctctcaaatt aaagctcaca ctgcctccat aaaagtcttc |
| 3241 | aacatctaat ttataagctt tacaagtatt tattttataa ggcttagaca gaattattgg |
| 3301 | agttttaaat taagtgtatt ggaaaagaaa ggatggtatg tgtatgaaat gttaagatcc |
| 3361 | tacgcaacac tgctattttt ttcctttaat atttgtgctg cataacaaaa gccactagac |
| 3421 | tgttactgtc ttgtctgtcc atgtgttaac agcatttctt aatgatgtat atatggagtg |
| 3481 | gtcttcaatc atagtgaaga atttaaagag aaagtcaatt gtattggcat ttttaataag |
| 3541 | aacaaaatta gttcgtctaa ggggactggc tggccacata tttgttcctt gcccatatgc |
| 3601 | tttctacttc ttgttcttat tatgaaatta tgaatttgaa gcctctgaaa tggtgatcag |
| 3661 | ttttcaacat ctttcaaaaa caaaattact atttcctcca tattgccttt tttagataac |
| 3721 | tttaaagtta ggattttaaa atatttgtaa ctggctaaat tttaaagtcg tgacaaataa |
| 3781 | ttacttaggt tcagaaatat acacacactt actctttagc cagtttcttt caaggtttac |
| 3841 | tgtcccatca gatatctagc cattttcctt tgcaaattac ataccttctt aagagtgtat |
| 3901 | ttttaagatt attacttacg ctttatgatg atatagtttt tcaaaattat ttatagcttc |
| 3961 | atatgatgtt ttgtaatttt ttctattgat acctgtttta aaaatatttt ccaaggaagt |
| 4021 | tgattaaaat tatatttgtt accttttaga aaaagcattg aaatgagttt ctcttgcttt |
| 4081 | ttcattttcc ctctgcttta tatgctcttc gcaatacatc atgtccaacg ggatacctat |
| 4141 | tgttctcatg acacccaaaa ttgatgagag caaaggggtc gcaccatatg gaaatgttga |
| 4201 | aaactattgt aaagtagtat tatgaagtag cttttgtgtc attcatgtcg atgacatgaa |
| 4261 | agtgaagtaa atttattcta tgtaaattca cactaaaacc agtacagtac cataagtaga |
| 4321 | atacatgtaa gaatcaccta gtcttcacta tattgagtaa atataacatg ctaattttac |
| 4381 | aattaatgaa actaaacttt taaacatctc cattatatct acatcctttt gaaggtattt |
| 4441 | atcatagttg ccaattttaa ttttaggatt gactttctct ttctgaatga cttcataaag |
| 4501 | tttggtgtga attttgaaga cttgggttac taatgattgt atctttgcta gtcaacaact |
| 4561 | tatgaaatat actcaatgcg tctgatgtgt cattaagtgc agaaataact aagacacaaa |
| 4621 | taacctttgc aaaccttcaa gctgtgtaat attccaatgt tgtttttttc tttgtatata |
| 4681 | tacttatatc acgtaggatg taaaaccagt atgaccttgt ctagtctcca aacttaaaat |
| 4741 | aaacttttga aaagctggga aaaaaaaaaa a |

EGFR
SEQ ID NO: 10

| | |
|---|---|
| 1 | ccccggcgca gcgcggccgc agcagcctcc gccccccgca cggtgtgagc gcccgacgcg |
| 61 | gccgaggcgg ccggagtccc gagctagccc cggcggccgc cgccgcccag accggacgac |
| 121 | aggccacctc gtcggcgtcc gcccgagtcc ccgcctcgcc gccaacgcca caaccaccgc |
| 181 | gcacggcccc ctgactccgt ccagtattga tcgggagagc cggagcgagc tcttcgggga |
| 241 | gcagcgatgc gaccctccgg gacggccggg gcagcgctcc tggcgctgct ggctgcgctc |
| 301 | tgcccggcga gtcgggctct ggaggaaaag aaagtttgcc aaggcacgag taacaagctc |
| 361 | acgcagttgg gcacttttga agatcatttt ctcagcctcc agaggatgtt caataactgt |
| 421 | gaggtggtcc ttgggaattt ggaaattacc tatgtgcaga ggaattatga tctttccttc |
| 481 | ttaaagacca tccaggaggt ggctggttat gtcctcattg ccctcaacac agtggagcga |
| 541 | attcctttgg aaaacctgca gatcatcaga ggaaatatgt actacgaaaa ttcctatgcc |
| 601 | ttagcagtct tatctaacta tgatgcaaat aaaaccggac tgaaggagct gcccatgaga |
| 661 | aatttacagg aaatcctgca tggcgccgtg cggttcagca acaaccctgc cctgtgcaac |
| 721 | gtggagagca tccagtggcg ggacatagtc agcagtgact ttctcagcaa catgtcgatg |
| 781 | gacttccaga accacctggg cagctgccaa aagtgtgatc caagctgtcc caatgggagc |
| 841 | tgctggggtg caggagagga gaactgccag aaactgacca aaatcatctg tgcccagcag |
| 901 | tgctccgggc gctgccgtgg caagtccccc agtgactgct gccacaacca gtgtgctgca |
| 961 | ggctgcacag gcccccggga gagcgactgc ctggtctgcc gcaaattccg agacgaagcc |
| 1021 | acgtgcaagg acacctgccc cccactcatg ctctacaacc ccaccacgta ccagatggat |
| 1081 | gtgaaccccg agggcaaata cagctttggt gccacctgcg tgaagaagtg tccccgtaat |
| 1141 | tatgtggtga cagatcacgg ctcgtgcgtc cgagcctgtg gggccgacag ctatgagatg |
| 1201 | gaggaagacg gcgtccgcaa gtgtaagaag tgcgaagggc cttgccgcaa agtgtgtaac |
| 1261 | ggaataggta ttggtgaatt taaagactca ctctccataa atgctacgaa tattaaacac |
| 1321 | ttcaaaaact gcacctccat cagtggcgat ctccacatcc tgccggtggc atttaggggt |
| 1381 | gactccttca cacatactcc tcctctggat ccacaggaac tggatattct gaaaaccgta |
| 1441 | aaggaaatca cagggttttt gctgattcag gcttggcctg aaaacaggac ggacctccat |
| 1501 | gcctttgaga acctagaaat catacgcggc aggaccaagc aacatggtca gttttctctt |
| 1561 | gcagtcgtca gcctgaacat aacatccttg ggattacgct ccctcaagga gataagtgat |
| 1621 | ggagatgtga taatttcagg aaacaaaaat ttgtgctatg caaatacaat aaactggaaa |
| 1681 | aaactgtttg ggacctccgg tcagaaaacc aaaattataa gcaacagagg tgaaaacagc |
| 1741 | tgcaaggcca caggccaggt ctgccatgcc ttgtgctccc ccgagggctg ctggggcccg |
| 1801 | gagcccaggg actgcgtctc ttgccggaat gtcagccgag gcagggaatg cgtggacaag |
| 1861 | tgcaaccttc tggagggtga gccaagggag tttgtggaga actctgagtg catacagtgc |
| 1921 | cacccagagt gcctgcctca ggccatgaac atcacctgca caggacgggg accagacaac |
| 1981 | tgtatccagt gtgcccacta cattgacggc ccccactgcg tcaagacctg cccggcagga |
| 2041 | gtcatgggag aaaacaacac cctggtctgg aagtacgcag acgccggcca tgtgtgccac |
| 2101 | ctgtgccatc caaactgcac ctacggatgc actgggccag gtcttgaagg ctgtccaacg |
| 2161 | aatgggccta agatcccgtc catcgccact gggatggtgg gggccctcct cttgctgctg |
| 2221 | gtggtggccc tggggatcgg cctcttcatg cgaaggcgcc acatcgttcg gaagcgcacg |
| 2281 | ctgcggaggc tgctgcagga gagggagctt gtggagcctc ttacacccag tggagaagct |
| 2341 | cccaaccaag ctctcttgag gatcttgaag gaaactgaat tcaaaaagat caaagtgctg |
| 2401 | ggctccggtg cgttcggcac ggtgtataag ggactctgga tcccagaagg tgagaaagtt |
| 2461 | aaaattcccg tcgctatcaa ggaattaaga gaagcaacat ctccgaaagc caacaaggaa |
| 2521 | atcctcgatg aagcctacgt gatggccagc gtggacaacc cccacgtgtg ccgcctgctg |
| 2581 | ggcatctgcc tcacctccac cgtgcagctc atcacgcagc tcatgccctt cggctgcctc |
| 2641 | ctggactatg tccgggaaca caaagacaat attggctccc agtacctgct caactggtgt |
| 2701 | gtgcagatcg caaagggcat gaactacttg gaggaccgtc gcttggtgca ccgcgacctg |
| 2761 | gcagccagga acgtactggt gaaaacaccg cagcatgtca agatcacaga ttttgggctg |
| 2821 | gccaaactgc tgggtgcgga agagaaagaa taccatgcag aaggaggcaa agtgcctatc |
| 2881 | aagtggatgg cattggaatc aattttacac agaatctata cccaccagag tgatgtctgg |
| 2941 | agctacgggg tgaccgtttg ggagttgatg acctttggat ccaagccata tgacggaatc |
| 3001 | cctgccagcg agatctcctc catcctggag aaaggagaac gcctccctca gccacccata |
| 3061 | tgtaccatcg atgtctacat gatcatggtc aagtgctgga tgatagacgc agatagtcgc |
| 3121 | ccaaagttcc gtgagttgat catcgaattc tccaaaatgg cccgagaccc ccagcgctac |
| 3181 | cttgtcattc agggggatga aagaatgcat ttgccaagtc ctacagactc caacttctac |
| 3241 | cgtgccctga tggatgaaga agacatggac gacgtggtgg atgccgacga gtacctcatc |
| 3301 | ccacagcagg gcttcttcag cagcccctcc acgtcacgga ctcccctcct gagctctctg |
| 3361 | agtgcaacca gcaacaattc caccgtggct tgcattgata gaaatgggct gcaaagctgt |
| 3421 | cccatcaagg aagacagctt cttgcagcga tacagctcag accccacagg cgccttgact |
| 3481 | gaggacagca tagacgacac cttcctccca gtgcctgaat acataaacca gtccgttccc |
| 3541 | aaaaggcccg ctggctctgt gcagaatcct gtctatcaca atcagcctct gaaccccgcg |
| 3601 | cccagcagag acccacacta ccaggacccc cacagcactg cagtgggcaa ccccgagtat |
| 3661 | ctcaacactg tccagcccac ctgtgtcaac agcacattcg acagccctgc ccactgggcc |
| 3721 | cagaaaggca gccaccaaat tagcctggac aaccctgact accagcagga cttctttccc |
| 3781 | aaggaagcca agccaaatgg catctttaag ggctccacag ctgaaaatgc agaataccta |
| 3841 | agggtcgcgc cacaaagcag tgaatttatt ggagcatgac cacggaggat agtatgagcc |
| 3901 | ctaaaaatcc agactctttc gatacccagg accaagccac agcaggtcct ccatcccaac |
| 3961 | agccatgccc gcattagctc ttagacccac agactggttt tgcaacgttt acaccgacta |
| 4021 | gccaggaagt acttccacct cgggcacatt ttgggaagtt gcattccttt gtcttcaaac |
| 4081 | tgtgaagcat ttacagaaac gcatccagca agaatattgt ccctttgagc agaaatttat |
| 4141 | ctttcaaaga ggtatatttg aaaaaaaaaa aaagtatatg tgaggatttt tattgattgg |
| 4201 | ggatcttgga gtttttcatt gtcgctattg atttttactt caatgggctc ttccaacaag |
| 4261 | gaagaagctt gctggtagca cttgctaccc tgagttcatc caggcccaac tgtgagcaag |
| 4321 | gagcacaagc cacaagtctt ccagaggatg cttgattcca gtggttctgc ttcaaggctt |
| 4381 | ccactgcaaa acactaaaga tccaagaagg ccttcatggc cccagcaggc cggatcggta |
| 4441 | ctgtatcaag tcatggcagg tacagtagga taagccactc tgtcccttcc tgggcaaaga |
| 4501 | agaaacggag gggatggaat tcttccttag acttactttt gtaaaaatgt ccccacggta |
| 4561 | cttactcccc actgatggac cagtggtttc cagtcatgag cgttagactg acttgtttgt |
| 4621 | cttccattcc attgttttga aactcagtat gctgcccctg tcttgctgtc atgaaatcag |
| 4681 | caagagagga tgacacatca aataataact cggattccag cccacattgg attcatcagc |
| 4741 | atttggacca atagcccaca gctgagaatg tggaatacct aaggatagca ccgcttttgt |
| 4801 | tctcgcaaaa acgtatctcc taatttgagg ctcagatgaa atgcatcagg tcctttgggg |
| 4861 | catagatcag aagactacaa aaatgaagct gctctgaaat ctcctttagc catcacccca |
| 4921 | accccccaaa attagtttgt gttacttatg gaagatagtt ttctcctttt acttcacttc |
| 4981 | aaaagctttt tactcaaaga gtatatgttc cctccaggtc agctgccccc aaaccccctc |
| 5041 | cttacgcttt gtcacacaaa aagtgtctct gccttgagtc atctattcaa gcacttacag |
| 5101 | ctctggccac aacagggcat tttacaggtg cgaatgacag tagcattatg agtagtgtgg |
| 5161 | aattcaggta gtaaatatga aactagggtt tgaaattgat aatgctttca caacatttgc |
| 5221 | agatgtttta gaaggaaaaa agttccttcc taaaataatt tctctacaat tggaagattg |
| 5281 | gaagattcag ctagttagga gcccaccttt tttcctaatc tgtgtgtgcc ctgtaacctg |
| 5341 | actggttaac agcagtcctt tgtaaacagt gttttaaact ctcctagtca atatccaccc |
| 5401 | catccaattt atcaaggaag aaatggttca gaaaatattt tcagcctaca gttatgttca |
| 5461 | gtcacacaca catacaaaat gttccttttg cttttaaagt aatttttgac tcccagatca |
| 5521 | gtcagagccc ctacagcatt gttaagaaag tatttgattt ttgtctcaat gaaaataaaa |
| 5581 | ctatattcat ttccactcta aaaaaaaaaa aaaaaa |

FGFR
SEQ ID NO: 11

| | |
|---|---|
| 1 | gccacaggcg cggcgtcctc ggcggcgggc ggcagctagc gggagccggg acgccggtgc |
| 61 | agccgcagcg cgcggaggaa cccgggtgtg ccgggagctg ggcggccacg tccggtcggg |
| 121 | accgagaccc ctcgtagcgc attgcggcga cctcgccttc cccggccgcg agcgcgccgc |
| 181 | tgcttgaaaa gccgcggaac ccaaggactt ttctccggtc cgagctcggg gcgccccgca |
| 241 | ggcgcacggt acccgtgctg cagctgggca cgccgcggcg ccggggcctc cgcaggcgcc |
| 301 | ggcctgcgtt ctggaggagg ggggcacaag gtctggagac cccgggtggc ggacgggagc |
| 361 | cctccccccg ccccgcctcc gcgaccagct ccgctccatt gttcccgccc ggctggaggc |
| 421 | gccgagcacc gagcgcgccg ggagtcgagc gccggccgcg agctcttgcg accccgccag |
| 481 | acccgaacag agcccggggg ccggcgcgga gccgggacgc gggcacacgg cctcgcacaa |
| 541 | gccacgggca ctctcccgag gcggaacctc cacgccgagc gagggtcagt ttgaaaagga |
| 601 | ggatcgagct cactgtggag tatccatgga gatgtggagc cttgtcacca acctctaact |
| 661 | gcagaactgg gatgtggagc tggaagtgcc tcctcttctg ggctgtgctg gtcacagcca |
| 721 | cactctgcac cgctaggccg tccccgacct tgcctgaaca agcccagccc tggggagccc |
| 781 | ctgtggaagt ggagtccttc ctggtccacc ccggtgacct gctgcagctt cgctgtcggc |
| 841 | tgcgggacga tgtgcagagc atcaactggc tgcgggacgg ggtgcagctg gcggaaagca |
| 901 | accgcacccg catcacaggg gaggaggtgg aggtgcagga ctccgtgccc gcagactccg |
| 961 | gcctctatgc ttgcgtaacc agcagcccct ccggaagtga caccacctac ttctccgtca |
| 1021 | atgtttcaga tgctctcccc tcctcggagg atgatgatga tgatgatgac tcctcttcag |
| 1081 | aggagaaaga aacagataac accaaaccaa accccgtagc tccatattgg acatccccag |
| 1141 | aaaagatgga aaagaaattg catgcagtgc cggctgccaa gacagtgaag ttcaaatgcc |
| 1201 | cttccagtgg gaccccaaac cccacactgc gctggttgaa aaatggcaaa gaattcaaac |
| 1261 | ctgaccacag aattggaggc tacaaggtcc gttatgccac ctggagcatc ataatggact |
| 1321 | ctgtggtgcc ctctgacaag ggcaactaca cctgcattgt ggagaatgag tacggcagca |
| 1381 | tcaaccacac ataccagctg gatgtcgtgg agcggtcccc tcaccgcccc atcctgcaag |
| 1441 | cagggttgcc cgccaacaaa acagtggccc tgggtagcaa cgtggagttc atgtgtaagg |
| 1501 | tgtacagtga cccgcagccg cacatccagt ggctaaagca catcgaggtg aatgggagca |
| 1561 | agattggccc agacaacctg ccttatgtcc agatcttgaa gactgctgga gttaatacca |
| 1621 | ccgacaaaga gatggaggtg cttcacttaa gaaatgtctc ctttgaggac gcaggggagt |
| 1681 | atacgtgctt ggcgggtaac tctatcggac tctcccatca ctctgcatgg ttgaccgttc |
| 1741 | tggaagccct ggaagagagg ccggcagtga tgacctcgcc cctgtacctg gagatcatca |
| 1801 | tctattgcac aggggccttc ctcatctcct gcatggtggg gtcggtcatc gtctacaaga |
| 1861 | tgaagagtgg taccaagaag agtgacttcc acagccagat ggctgtgcac aagctggcca |
| 1921 | agagcatccc tctgcgcaga caggtaacag tgtctgctga ctccagtgca tccatgaact |
| 1981 | ctggggttct tctggttcgg ccatcacggc tctcctccag tgggactccc atgctagcag |
| 2041 | gggtctctga gtatgagctt cccgaagacc ctcgctggga gctgcctcgg gacagactgg |
| 2101 | tcttaggcaa acccctggga gagggctgct ttgggcaggt ggtgttggca gaggctatcg |
| 2161 | ggctggacaa ggacaaaccc aaccgtgtga ccaaagtggc tgtgaagatg ttgaagtcgg |
| 2221 | acgcaacaga gaaagacttg tcagacctga tctcagaaat ggagatgatg aagatgatcg |
| 2281 | ggaagcataa gaatatcatc aacctgctgg gggcctgcac gcaggatggt cccttgtatg |
| 2341 | tcatcgtgga gtatgcctcc aagggcaacc tgcgggagta cctgcaggcc cggaggcccc |
| 2401 | cagggctgga atactgctac aaccccagcc acaacccaga ggagcagctc tcctccaagg |
| 2461 | acctggtgtc ctgcgcctac caggtggccc gaggcatgga gtatctggcc tccaagaagt |
| 2521 | gcatacaccg agacctggca gccaggaatg tcctggtgac agaggacaat gtgatgaaga |
| 2581 | tagcagactt tggcctcgca cgggacattc accacatcga ctactataaa aagacaacca |
| 2641 | acggccgact gcctgtgaag tggatggcac ccgaggcatt atttgaccgg atctacaccc |
| 2701 | accagagtga tgtgtggtct ttcggggtgc tcctgtggga gatcttcact ctgggcggct |
| 2761 | ccccataccc cggtgtgcct gtggaggaac ttttcaagct gctgaaggag ggtcaccgca |
| 2821 | tggacaagcc cagtaactgc accaacgagc tgtacatgat gatgcgggac tgctggcatg |
| 2881 | cagtgccctc acagagaccc accttcaagc agctggtgga agacctggac cgcatcgtgg |
| 2941 | ccttgacctc caaccaggag tacctggacc tgtccatgcc cctggaccag tactccccca |
| 3001 | gctttcccga cacccggagc tctacgtgct cctcagggga ggattccgtc ttctctcatg |
| 3061 | agccgctgcc cgaggagccc tgcctgcccc gacacccagc ccagcttgcc aatggcggac |
| 3121 | tcaaacgccg ctgactgcca cccacacgcc ctccccagac tccaccgtca gctgtaaccc |
| 3181 | tcacccacag cccctgcctg ggcccaccac ctgtccgtcc ctgtcccctt tcctgctggc |
| 3241 | aggagccggc tgcctacagg ggccttcctg tgtggcctgc cttcacccca ctcagctcac |
| 3301 | ctctccctcc acctcctctc cacctgctgg tgagaggtgc aaagaggcag atctttgctg |
| 3361 | ccagccactt catcccctcc cagatgttgg accaacaccc ctccctgcca ccaggcactg |
| 3421 | cctgagggca gggagtggga gccaatgaac aggcatgcaa gtgagagctt cctgagcttt |
| 3481 | ctcctgtcgg tttggtctgt tttgccttca cccataagcc cctcgcactc tggtggcagg |
| 3541 | tgcttgtcct cagggctaca gcagtaggga ggtcagtgct tcgagccacg attgaaggtg |
| 3601 | acctctgccc cagataggtg gtgccagtgg cttattaatt ccgatactag tttgctttgc |
| 3661 | tgaccaaatg cctggtacca gaggatggtg aggcgaaggc aggttggggg cagtgttgtg |
| 3721 | gcctggggcc agccaacact ggggctctgt atatagctat gaagaaaaca caaagttgat |
| 3781 | aaatctgagt atatatttac atgtcttttt aaaagggtcg ttaccagaga tttacccatc |
| 3841 | ggtaagatgc tcctggtggc tgggaggcat cagttgctat atattaaaaa caaaaaaaaa |
| 3901 | a |

FN1
SEQ ID NO: 12

| | |
|---|---|
| 1 | atcaaacaga aatgactatt gaaggcttgc agcccacagt ggagtatgtg gttagtgtct |
| 61 | atgctcagaa tccaagcgga gagagtcagc ctctggttca gactgcagta accaacattg |
| 121 | atcgccctaa aggactggca ttcactgatg tggatgtcga ttccatcaaa attgcttggg |
| 181 | aaagcccaca ggggcaagtt tccaggtaca gggtgaccta ctcgagccct gaggatggaa |
| 241 | tccatgagct attccctgca cctgatggtg aagaagacac tgcagagctg caaggcctca |
| 301 | gaccgggttc tgagtacaca gtcagtgtgg ttgccttgca cgatgatatg gagagccagc |
| 361 | ccctgattgg aacccagtcc acagctattc ctgcaccaac tgacctgaag ttcactcagg |
| 421 | tcacacccac aagcctgagc gcccagtgga caccacccaa tgttcagctc actggatatc |
| 481 | gagtgcgggt gacccccaag gagaagaccg gaccaatgaa agaaatcaac cttgctcctg |
| 541 | acagctcatc cgtggttgta tcaggactta tggtggccac caaatatgaa gtgagtgtct |
| 601 | atgctcttaa ggacactttg acaagcagac cagctcaggg tgttgtcacc actctggaga |
| 661 | atgtcagccc accaagaagg gctcgtgtga cagatgctac tgagaccacc atcaccatta |
| 721 | gctggagaac caagactgag acgatcactg gcttccaagt tgatgccgtt ccagccaatg |
| 781 | gccagactcc aatccagaga accatcaagc cagatgtcag aagctacacc atcacaggtt |
| 841 | tacaaccagg cactgactac aagatctacc tgtacacctt gaatgacaat gctcggagct |
| 901 | cccctgtggt catcgacgcc tccactgcca ttgatgcacc atccaacctg cgtttcctgg |
| 961 | ccaccacacc caattccttg ctggtatcat ggcagccgcc acgtgccagg attaccggct |
| 1021 | acatcatcaa gtatgagaag cctgggtctc ctcccagaga agtggtccct cggccccgcc |
| 1081 | ctggtgtcac agaggctact attactggcc tggaaccggg aaccgaatat acaatttatg |
| 1141 | tcattgccct gaagaataat cagaagagcg agcccctgat tggaaggaaa aagacagacg |
| 1201 | agcttcccca actggtaacc cttccacacc ccaatcttca tggaccagag atcttggatg |
| 1261 | ttccttccac agttcaaaag acccctttcg tcacccaccc tgggtatgac actggaaatg |
| 1321 | gtattcagct tcctggcact tctggtcagc aacccagtgt tgggcaacaa atgatctttg |
| 1381 | aggaacatgg ttttaggcgg accacaccgc ccacaacggc cacccccata aggcataggc |
| 1441 | caagaccata cccgccgaat gtaggtgagg aaatccaaat tggtcacatt cccagggaag |
| 1501 | atgtagacta tcacctgtac ccacacggtc cggggctcaa tccaaatgcc tctacaggac |
| 1561 | aagaagctct ctctcagaca accatctcat gggccccatt ccaggacact tctgagtaca |
| 1621 | tcatttcatg tcatcctgtt ggcactgatg aagaaccctt acagttcagg gttcctggaa |
| 1681 | cttctaccag tgcgactctg acaggcctca ccagaggtgc cacctacaac atcatagtgg |
| 1741 | aggcactgaa agaccagcag aggcataagg ttcgggaaga ggttgttacc gtgggcaact |
| 1801 | ctgtcaacga aggcttgaac caacctacgg atgactcgtg ctttgacccc tacacagttt |
| 1861 | cccattatgc cgttggagat gagtgggaac gaatgtctga atcaggcttt aaactgttgt |
| 1921 | gccagtgctt aggctttgga agtggtcatt tcagatgtga ttcatctaga tggtgccatg |
| 1981 | acaatggtgt gaactacaag attggagaga agtgggaccg tcagggagaa aatggccaga |
| 2041 | tgatgagctg cacatgtctt gggaacggaa aaggagaatt caagtgtgac cctcatgagg |
| 2101 | caacgtgtta cgatgatggg aagacatacc acgtaggaga acagtggcag aaggaatatc |
| 2161 | tcggtgccat ttgctcctgc acatgctttg gaggccagcg gggctggcgc tgtgacaact |
| 2221 | gccgcagacc tgggggtgaa cccagtcccg aaggcactac tggccagtcc tacaaccagt |
| 2281 | attctcagag ataccatcag agaacaaaca ctaatgttaa ttgcccaatt gagtgcttca |
| 2341 | tgcctttaga tgtacaggct gacagagaag attcccgaga gtaa |

MFGE8
SEQ ID NO: 13

| | |
|---|---|
| 1 | agtccgcctc tggccagctt gggcggagcg cacggccagt gggaggtgct gagccgcctg |
| 61 | atttattccg gtcccagagg agaaggcgcc agaaccccgc ggggtctgag cagcccagcg |
| 121 | tgcccattcc agcgcccgcg tccccgcagc atgccgcgcc cccgcctgct ggccgcgctg |
| 181 | tgcggcgcgc tgctctgcgc ccccagcctc ctcgtcgccc tggatatctg ttccaaaaac |
| 241 | ccctgccaca acggtggttt atgcgaggag atttcccaag aagtgcgagg agatgtcttc |
| 301 | ccctcgtaca cctgcacgtg ccttaagggc tacgcgggca accactgtga gacgaaatgt |
| 361 | gtcgagccac tgggcctgga gaatgggaac attgccaact cacagatcgc cgcctcgtct |
| 421 | gtgcgtgtga ccttcttggg tttgcagcat tgggtcccgg agctggcccg cctgaaccgc |
| 481 | gcaggcatgg tcaatgcctg gacacccagc agcaatgacg ataacccctg gatccaggtg |
| 541 | aacctgctgc ggaggatgtg ggtaacaggt gtggtgacgc agggtgccag ccgcttggcc |
| 601 | agtcatgagt acctgaaggc cttcaaggtg gcctacagcc ttaatggaca cgaattcgat |
| 661 | ttcatccatg atgttaataa aaaacacaag gagtttgtgg gtaactggaa caaaaacgcg |
| 721 | gtgcatgtca acctgtttga gacccctgtg gaggctcagt acgtgagatt gtaccccacg |
| 781 | agctgccaca cggcctgcac tctgcgcttt gagctactgg gctgtgagct gaacggatgc |
| 841 | gccaatcccc tgggcctgaa gaataacagc atccctgaca agcagatcac ggcctccagc |
| 901 | agctacaaga cctggggctt gcatctcttc agctggaacc cctcctatgc acggctggac |
| 961 | aagcagggca acttcaacgc ctgggttgcg gggagctacg gtaacgatca gtggctgcag |
| 1021 | gtggacctgg gctcctcgaa ggaggtgaca ggcatcatca cccagggggc ccgtaacttt |
| 1081 | ggctctgtcc agtttgtggc atcctacaag gttgcctaca gtaatgacag tgcgaactgg |
| 1141 | actgagtacc aggaccccag gactggcagc agtaagatct tccctggcaa ctgggacaac |
| 1201 | cactcccaca agaagaactt gtttgagacg cccatcctgg ctcgctatgt gcgcatcctg |
| 1261 | cctgtagcct ggcacaaccg catcgccctg cgcctggagc tgctgggctg ttagtggcca |
| 1321 | cctgccaccc ccaggtcttc ctgctttcca tgggcccgct gcctcttggc ttctcagccc |
| 1381 | ctttaaatca ccatagggct ggggactggg gaaggggagg gtgttcagag gcagcaccac |
| 1441 | cacacagtca cccctccctc cctctttccc accctccacc tctcacgggc cctgccccag |
| 1501 | cccctaagcc ccgtccccta acccccagtc ctcactgtcc tgttttctta ggcactgagg |
| 1561 | gatctgagta ggtctgggat ggacaggaaa gggcaaagta gggcgtgtgg tttccctgcc |
| 1621 | cctgtccgga ccgccgatcc caggtgcgtg tgtctctgtc tctcctagcc cctctctcac |
| 1681 | acatcacatt cccatggtgg cctcaagaaa ggcccggaag cgccaggctg gagataacag |
| 1741 | cctcttgccc gtcggccctg cgtcggccct ggggtaccat gtggccacaa ctgctgtggc |
| 1801 | cccctgtccc caagacactt ccccttgtct ccctggttgc ctctcttgcc ccttgtcctg |
| 1861 | aagcccagcg acacagaagg gggtggggcg ggtctatggg gagaaaggga gcgaggtcag |
| 1921 | aggagggcat gggttggcag ggtgggcgtt tggggccctc tatgctggct tttcacccca |
| 1981 | gaggacacag gcagcttcca aaatatattt atcttcttca cgggaaaaaa aaaaaaaaaa |
| 2041 | aa |

LGALS3BP
SEQ ID NO: 14

| | |
|---|---|
| 1 | aatcgaaagt agactctttt ctgaagcatt tcctgggatc agcctgacca cgctccatac |
| 61 | tgggagaggc ttctgggtca aaggaccagt ctgcagaggg atcctgtggc tggaagcgag |
| 121 | gaggctccac acggccgttg cagctaccgc agccaggatc tgggcatcca ggcacggcca |
| 181 | tgacccctcc gaggctcttc tgggtgtggc tgctggttgc aggaacccaa ggcgtgaacg |
| 241 | atggtgacat gcggctggcc gatgggggcg ccaccaacca gggccgcgtg gagatcttct |
| 301 | acagaggcca gtggggcact gtgtgtgaca acctgtggga cctgactgat gccagcgtcg |
| 361 | tctgccgggc cctgggcttc gagaacgcca cccaggctct gggcagagct gccttcgggc |
| 421 | aaggatcagg ccccatcatg ctggatgagg tccagtgcac gggaaccgag gcctcactgg |
| 481 | ccgactgcaa gtccctgggc tggctgaaga gcaactgcag gcacgagaga gacgctggtg |
| 541 | tggtctgcac caatgaaacc aggagcaccc acaccctgga cctctccagg gagctctcgg |
| 601 | aggcccttgg ccagatcttt gacagccagc ggggctgcga cctgtccatc agcgtgaatg |
| 661 | tgcagggcga ggacgccctg ggcttctgtg gccacacggt catcctgact gccaacctgg |
| 721 | aggcccaggc cctgtggaag gagccgggca gcaatgtcac catgagtgtg gatgctgagt |
| 781 | gtgtgcccat ggtcagggac cttctcaggt acttctactc ccgaaggatt gacatcaccc |
| 841 | tgtcgtcagt caagtgcttc cacaagctgg cctctgccta tggggccagg cagctgcagg |
| 901 | gctactgcgc aagcctcttt gccatcctcc tcccccagga cccctcgttc cagatgcccc |
| 961 | tggacctgta tgcctatgca gtggccacag gggacgccct gctggagaag ctctgcctac |
| 1021 | agttcctggc ctggaacttc gaggccttga cgcaggccga ggcctggccc agtgtcccca |
| 1081 | cagacctgct ccaactgctg ctgcccagga gcgacctggc ggtgcccagc gagctggccc |
| 1141 | tactgaaggc cgtggacacc tggagctggg gggagcgtgc ctcccatgag gaggtggagg |
| 1201 | gcttggtgga gaagatccgc ttccccatga tgctccctga ggagctcttt gagctgcagt |
| 1261 | tcaacctgtc cctgtactgg agccacgagg ccctgttcca gaagaagact ctgcaggccc |
| 1321 | tggaattcca cactgtgccc ttccagttgc tggcccggta caaaggcctg aacctcaccg |
| 1381 | aggataccta caagccccgg atttacacct cgcccacctg gagtgccttt gtgacagaca |
| 1441 | gttcctggag tgcacggaag tcacaactgg tctatcagtc cagacggggg cctttggtca |
| 1501 | aatattcttc tgattacttc caagccccct ctgactacag atactacccc taccagtcct |
| 1561 | tccagactcc acaacacccc agcttcctct tccaggacaa gagggtgtcc tggtccctgg |
| 1621 | tctacctccc caccatccag agctgctgga actacggctt ctcctgctcc tcggacgagc |
| 1681 | tccctgtcct gggcctcacc aagtctggcg gctcagatcg caccattgcc tacgaaaaca |
| 1741 | aagccctgat gctctgcgaa gggctcttcg tggcagacgt caccgatttc gagggctgga |
| 1801 | aggctgcgat tcccagtgcc ctggacacca acagctcgaa gagcacctcc tccttcccct |
| 1861 | gcccggcagg gcacttcaac ggcttccgca cggtcatccg ccccttctac ctgaccaact |
| 1921 | cctcaggtgt ggactagacg gcgtggccca agggtggtga gaaccggaga accccaggac |
| 1981 | gccctcactg caggctcccc tcctcggctt ccttcctctc tgcaatgacc ttcaacaacc |
| 2041 | ggccaccaga tgtcgcccta ctcacctgag cgctcagctt caagaaatta ctggaaggct |
| 2101 | tccactaggg tccaccagga gttctcccac cacctcacca gtttccaggt ggtaagcacc |
| 2161 | aggacgccct cgaggttgct ctgggatccc cccacagccc ctggtcagtc tgcccttgtc |
| 2221 | actggtctga ggtcattaaa attacattga ggttcctaca aaaaaaaaaa aaaaaaa |

TF
SEQ ID NO: 15

| | |
|---|---|
| 1 | tgtgctcgct gctcagcgcg cacccggaag atgaggctcg ccgtgggagc cctgctggtc |
| 61 | tgcgccgtcc tggggctgtg tctggctgtc cctgataaaa ctgtgagatg gtgtgcagtg |
| 121 | tcggagcatg aggccactaa gtgccagagt ttccgcgacc atatgaaaag cgtcattcca |
| 181 | tccgatggtc ccagtgttgc ttgtgtgaag aaagcctcct accttgattg catcagggcc |
| 241 | attgcggcaa acgaagcgga tgctgtgaca ctggatgcag gtttggtgta tgatgcttac |
| 301 | ttggctccca ataacctgaa gcctgtggtg gcagagttct atgggtcaaa agaggatcca |
| 361 | cagactttct attatgctgt tgctgtggtg aagaaggata gtggcttcca gatgaaccag |
| 421 | cttcgaggca agaagtcctg ccacacgggt ctaggcaggt ccgctgggtg gaacatcccc |
| 481 | ataggcttac tttactgtga cttacctgag ccacgtaaac ctcttgagaa agcagtggcc |
| 541 | aatttcttct cgggcagctg tgccccttgt gcggatggga cggacttccc ccagctgtgt |
| 601 | caactgtgtc cagggtgtgg ctgctccacc cttaaccaat acttcggcta ctcgggagcc |
| 661 | ttcaagtgtc tgaaggatgg tgctggggat gtggcctttg tcaagcactc gactatattt |
| 721 | gagaacttgg caaacaaggc tgacagggac cagtatgagc tgctttgcct agacaacacc |
| 781 | cggaagccgg tagatgaata caaggactgc cacttggccc aggtcccttc tcataccgtc |
| 841 | gtggcccgaa gtatgggcgg caaggaggac ttgatctggg agcttctcaa ccaggcccag |
| 901 | gaacattttg gcaaagacaa atcaaaagaa ttccaactat tcagctctcc tcatgggaag |
| 961 | gacctgctgt ttaaggactc tgcccacggg tttttaaaag tccccccaag gatggatgcc |
| 1021 | aagatgtacc tgggctatga gtatgtcact gccatccgga atctacggga aggcacatgc |
| 1081 | ccagaagccc caacagatga atgcaagcct gtgaagtggt gtgcgctgag ccaccacgag |
| 1141 | aggctcaagt gtgatgagtg gagtgttaac agtgtaggga aaatagagtg tgtatcagca |
| 1201 | gagaccaccg aagactgcat cgccaagatc atgaatggag aagctgatgc catgagcttg |
| 1261 | gatggagggt ttgtctacat agcgggcaag tgtggtctgg tgcctgtctt ggcagaaaac |
| 1321 | tacaataaga gcgataattg tgaggataca ccagaggcag ggtattttgc tgtagcagtg |
| 1381 | gtgaagaaat cagcttctga cctcacctgg gacaatctga aaggcaagaa gtcctgccat |
| 1441 | acggcagttg gcagaaccgc tggctggaac atccccatgg gcctgctcta caataagatc |
| 1501 | aaccactgca gatttgatga atttttcagt gaaggttgtg cccctgggtc taagaaagac |
| 1561 | tccagtctct gtaagctgtg tatgggctca ggcctaaacc tgtgtgaacc caacaacaaa |
| 1621 | gagggatact acggctacac aggcgctttc aggtgtctgg ttgagaaggg agatgtggcc |
| 1681 | tttgtgaaac accagactgt cccacagaac actgggggaa aaaaccctga tccatgggct |
| 1741 | aagaatctga atgaaaaaga ctatgagttg ctgtgccttg atggtaccag gaaacctgtg |
| 1801 | gaggagtatg cgaactgcca cctggccaga gccccgaatc acgctgtggt cacacggaaa |
| 1861 | gataaggaag cttgcgtcca caagatatta cgtcaacagc agcacctatt tggaagcaac |
| 1921 | gtaactgact gctcgggcaa cttttgtttg ttccggtcgg aaaccaagga ccttctgttc |
| 1981 | agagatgaca cagtatgttt ggccaaactt catgacagaa acacatatga aaaatactta |
| 2041 | ggagaagaat atgtcaaggc tgttggtaac ctgagaaaat gctccacctc atcactcctg |
| 2101 | gaagcctgca ctttccgtag accttaaaat ctcagaggta gggctgccac caaggtgaag |
| 2161 | atgggaacgc agatgatcca tgagtttgcc ctggtttcac tggcccaagt ggtttgtgct |
| 2221 | aaccacgtct gtcttcacag ctctgtgttg ccatgtgtgc tgaacaaaaa ataaaaatta |
| 2281 | ttattgattt tatatttc |

VEGFR
SEQ ID NO: 16

| | |
|---|---|
| 1 | atggtcagct actgggacac cggggtcctg ctgtgcgcgc tgctcagctg tctgcttctc |
| 61 | acaggatcta gttcaggttc aaaattaaaa gatcctgaac tgagtttaaa aggcacccag |
| 121 | cacatcatgc aagcaggcca gacactgcat ctccaatgca ggggggaagc agcccataaa |
| 181 | tggtctttgc ctgaaatggt gagtaaggaa agcgaaaggc tgagcataac taaatctgcc |
| 241 | tgtggaagaa atggcaaaca attctgcagt actttaacct tgaacacagc tcaagcaaac |
| 301 | cacactggct tctacagctg caaatatcta gctgtaccta cttcaaagaa gaaggaaaca |
| 361 | gaatctgcaa tctatatatt tattagtgat acaggtagac ctttcgtaga gatgtacagt |
| 421 | gaaatccccg aaattataca catgactgaa ggaagggagc tcgtcattcc ctgccgggtt |
| 481 | acgtcaccta acatcactgt tactttaaaa aagtttccac ttgacacttt gatccctgat |
| 541 | ggaaaacgca taatctggga cagtagaaag ggcttcatca tatcaaatgc aacgtacaaa |
| 601 | gaaatagggc ttctgacctg tgaagcaaca gtcaatgggc atttgtataa gacaaactat |
| 661 | ctcacacatc gacaaaccaa tacaatcata gatgtccaaa taagcacacc acgcccagtc |
| 721 | aaattactta gaggccatac tcttgtcctc aattgtactg ctaccactcc cttgaacacg |
| 781 | agagttcaaa tgacctggag ttaccctgat gaaaaaaata agagagcttc cgtaaggcga |
| 841 | cgaattgacc aaagcaattc ccatgccaac atattctaca gtgttcttac tattgacaaa |
| 901 | atgcagaaca aagacaaagg actttatact tgtcgtgtaa ggagtggacc atcattcaaa |
| 961 | tctgttaaca cctcagtgca tatatatgat aaagcattca tcactgtgaa acatcgaaaa |
| 1021 | cagcaggtgc ttgaaaccgt agctggcaag cggtcttacc ggctctctat gaaagtgaag |
| 1081 | gcatttccct cgccggaagt tgtatggtta aaagatgggt tacctgcgac tgagaaatct |
| 1141 | gctcgctatt tgactcgtgg ctactcgtta attatcaagg acgtaactga agaggatgca |
| 1201 | gggaattata caatcttgct gagcataaaa cagtcaaatg tgtttaaaaa cctcactgcc |
| 1261 | actctaattg tcaatgtgaa accccagatt tacgaaaagg ccgtgtcatc gtttccagac |
| 1321 | ccggctctct acccactggg cagcagacaa atcctgactt gtaccgcata tggtatccct |
| 1381 | caacctacaa tcaagtggtt ctggcacccc tgtaaccata atcattccga agcaaggtgt |
| 1441 | gacttttgtt ccaataatga agagtcctct atcctggatg ctgacagcaa catgggaaac |
| 1501 | agaattgaga gcatcactca gcgcatggca ataatagaag gaaagaataa gatggctagc |
| 1561 | accttggttg tggctgactc tagaatttct ggaatctaca tttgcatagc ttccaataaa |
| 1621 | gttgggactg tgggaagaaa cataagcttt tatatcacag atgtgccaaa tgggtttcat |
| 1681 | gttaacttgg aaaaaatgcc gacggaagga gaggacctga aactgtcttg cacagttaac |
| 1741 | aagttcttat acagagacgt tacttggatt ttactgcgga cagttaataa cagaacaatg |
| 1801 | cactacagta ttagcaagca aaaaatggcc atcactaagg agcactccat cactcttaat |
| 1861 | cttaccatca tgaatgtttc cctgcaagat tcaggcacct atgcctgcag agccaggaat |
| 1921 | gtatacacag gggaagaaat cctccagaag aaagaaatta caatcagaga tcaggaagca |
| 1981 | ccatacctcc tgcgaaacct cagtgatcac acagtggcca tcagcagttc caccacttta |
| 2041 | gactgtcatg ctaatggtgt ccccgagcct cagatcactt ggtttaaaaa caaccacaaa |
| 2101 | atacaacaag agcctggaat tattttagga ccaggaagca gcacgctgtt tattgaaaga |
| 2161 | gtcacagaag aggatgaagg tgtctatcac tgcaaagcca ccaaccagaa gggctctgtg |
| 2221 | gaaagttcag catacctcac tgttcaagga acctcggaca agtctaatct ggagctgatc |
| 2281 | actctaacat gcacctgtgt ggctgcgact ctcttctggc tcctattaac cctctttatc |
| 2341 | cgaaaaatga aaaggtcttc ttctgaaata aagactgact acctatcaat tataatggac |
| 2401 | ccagatgaag ttcctttgga tgagcagtgt gagcggctcc cttatgatgc cagcaagtgg |
| 2461 | gagtttgccc gggagagact taaactgggc aaatcacttg gaagaggggc ttttggaaaa |
| 2521 | gtggttcaag catcagcatt tggcattaag aaatcaccta cgtgccggac tgtggctgtg |
| 2581 | aaaatgctga aagagggggc cacggccagc gagtacaaag ctctgatgac tgagctaaaa |
| 2641 | atcttgaccc acattggcca ccatctgaac gtggttaacc tgctgggagc ctgcaccaag |
| 2701 | caaggagggc ctctgatggt gattgttgaa tactgcaaat atggaaatct ctccaactac |
| 2761 | ctcaagagca aacgtgactt attttttctc aacaaggatg cagcactaca catggagcct |
| 2821 | aagaaagaaa aaatggagcc aggcctggaa caaggcaaga aaccaagact agatagcgtc |
| 2881 | accagcagcg aaagctttgc gagctccggc tttcaggaag ataaaagtct gagtgatgtt |
| 2941 | gaggaagagg aggattctga cggtttctac aaggagccca tcactatgga agatctgatt |
| 3001 | tcttacagtt ttcaagtggc cagaggcatg gagttcctgt cttccagaaa gtgcattcat |
| 3061 | cgggacctgg cagcgagaaa cattctttta tctgagaaca acgtggtgaa gatttgtgat |
| 3121 | tttggccttg cccgggatat ttataagaac cccgattatg tgagaaaagg agatactcga |
| 3181 | cttcctctga aatggatggc tcctgaatct atctttgaca aaatctacag caccaagagc |
| 3241 | gacgtgtggt cttacggagt attgctgtgg gaaatcttct ccttaggtgg gtctccatac |
| 3301 | ccaggagtac aaatggatga ggacttttgc agtcgcctga gggaaggcat gaggatgaga |
| 3361 | gctcctgagt actctactcc tgaaatctat cagatcatgc tggactgctg gcacagagac |
| 3421 | ccaaaagaaa ggccaagatt tgcagaactt gtggaaaaac taggtgattt gcttcaagca |
| 3481 | aatgtacaac aggatggtaa agactacatc ccaatcaatg ccatactgac aggaaatagt |
| 3541 | gggtttacat actcaactcc tgccttctct gaggacttct tcaaggaaag tatttcagct |
| 3601 | ccgaagttta attcaggaag ctctgatgat gtcagatatg taaatgcttt caagttcatg |
| 3661 | agcctggaaa gaatcaaaac ctttgaagaa cttttaccga atgccacctc catgtttgat |
| 3721 | gactaccagg gcgacagcag cactctgttg gcctctccca tgctgaagcg cttcacctgg |
| 3781 | actgacagca aacccaaggc ctcgctcaag attgacttga gagtaaccag taaaagtaag |
| 3841 | gagtcggggc tgtctgatgt cagcaggccc agtttctgcc attccagctg tgggcacgtc |
| 3901 | agcgaaggca agcgcaggtt cacctacgac cacgctgagc tggaaaggaa aatcgcgtgc |
| 3961 | tgctccccgc ccccagacta caactcggtg gtcctgtact ccaccccacc catctag |

miR-132
SEQ ID NO: 17

| | |
|---|---|
| 1 | ccgcccccgc gtctccaggg caaccgtggc tttcgattgt tactgtggga actggaggta |
| 61 | acagtctaca gccatggtcg ccccgcagca cgcccacgcg c |

pCCLc-MNDU3c-MIR132-PGK-Tomato-WPRE
SEQ ID NO: 18

| | |
|---|---|
| Features | Nucleotide |
| MNDU3 promoter | 4661 .. 5204 |
| miR-132 | 5208 .. 5363 |
| PGK promoter | 5364 .. 5874 |
| td-Tomato | 5894 .. 7321 |
| WPRE | 7345 .. 7941 |

CAGGTGGCACTTTTCGGGGAAATGTGCGCGGAACCCCTATTTGTTTATTTTTCTAAATACATTCAAATATGTATC
CGCTCATGAGACAATAACCCTGATAAATGCTTCAATAATATTGAAAAAGGAAGAGTATGAGTATTCAACATTTCC
GTGTCGCCCTTATTCCCTTTTTTGCGGCATTTTGCCTTCCTGTTTTTGCTCACCCAGAAACGCTGGTGAAAGTAA
AAGATGCTGAAGATCAGTTGGGTGCACGAGTGGGTTACATCGAACTGGATCTCAACAGCGGTAAGATCCTTGAGAG
TTTTCGCCCCGAAGAACGTTTTCCAATGATGAGCACTTTTAAAGTTCTGCTATGTGGCGCGGTATTATCCCGTATTGACG
CCGGGCAAGAGCAACTCGGTCGCCGCATACACTATTCTCAGAATGACTTGGTTGAGTACTCACCAGTCACAGAAAAGCA
TCTTACGGATGGCATGACAGTAAGAGAATTATGCAGTGCTGCCATAACCATGAGTGATAACACTGCGGCCAACTTACTT
CTGACAACGATCGGAGGACCGAAGGAGCTAACCGCTTTTTTGCACAACATGGGGGATCATGTAACTCGCCTTGATCGTT
GGGAACCGGAGCTGAATGAAGCCATACCAAACGACGAGCGTGACACCACGATGCCTGTAGCAATGGCAACAACGTTGCG
CAAACTATTAACTGGCGAACTACTTACTCTAGCTTCCCGGCAACAATTAATAGACTGGATGGAGGCGGATAAAGTTGCA
GGACCACTTCTGCGCTCGGCCCTTCCGGCTGGCTGGTTTATTGCTGATAAATCTGGAGCCGGTGAGCGTGGGTCTCGCG
GTATCATTGCAGCACTGGGGCCAGATGGTAAGCCCTCCCGTATCGTAGTTATCTACACGACGGGGAGTCAGGCAACTAT
GGATGAACGAAATAGACAGATCGCTGAGATAGGTGCCTCACTGATTAAGCATTGGTAACTGTCAGACCAAGTTTACTCA
TATATACTTTAGATTGATTTAAAACTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAGATCAAAGGATCTTCTTGAGATCCTT
TTTTTCTGCGCGTAATCTGCTGCTTGCAAACAAAAAAACCACCGCTACCAGCGGTGGTTTGTTTGCCGGATCAAGAGCT
ACCAACTCTTTTTCCGAAGGTAACTGGCTTCAGCAGAGCGCAGATACCAAATACTGTCCTTCTAGTGTAGCCGTAGTTAG
GCCACCACTTCAAGAACTCTGTAGCACCGCCTACATACCTCGCTCTGCTAATCCTGTTACCAGTGGCTGCTGCCAGTGG
CGATAAGTCGTGTCTTACCGGGTTGGACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTACAGCGTGAGCTATGAGAAAGCGCCA
CGCTTCCCGAAGGGAGAAAGGCGGACAGGTATCCGGTAAGCGGCAGGGTCGGAACAGGAGAGCGCACGAGGGAGCTTCC
AGGGGGAAACGCCTGGTATCTTTATAGTCCTGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGGTTCCTGGCCTTTTGCTGGCCTTTTGCTCA
CATGTTCTTTCCTGCGTTATCCCCTGATTCTGTGGATAACCGTATTACCGCCTTTGAGTGAGCTGATACCGCTCGCCGCA
GCCGAACGACCGAGCGCAGCGAGTCAGTGAGCGAGGAAGCGGAAGAGCGCCCAATACGCAAACCGCCTCTCCCCGCGCG
TTGGCCGATTCATTAATGCAGCTGGCACGACAGGTTTCCCGACTGGAAAGCGGGCAGTGAGCGCAACGCAATTAATGTG
AGTTAGCTCACTCATTAGGCACCCCAGGCTTTACACTTTATGCTTCCGGCTCGTATGTTGTGTGGAATTGTGAGCGGATA
ACAATTTCACACAGGAAACAGCTATGACCATGATTACGCCAAGCGCGCAATTAACCCTCACTAAAGGGAACAAAAGCTG
GAGCTGCAAGCTTGGCCATTGCATACGTTGTATCCATATCATAATATGTACATTTATATTGGCTCATGTCCAACATTACC
GCCATGTTGACATTGATTATTGACTAGTTATTAATAGTAATCAATTACGGGGTCATTAGTTCATAGCCCATATATGGAGT
TCCGCGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCACTTGG
CAGTACATCAAGTGTATCATATGCCAAGTACGCCCCCTATTGACGTCAATGACGGTAAATGGCCCGCCTGGCATTATGC
CCAGTACATGACCTTATGGGACTTTCCTACTTGGCAGTACATCTACGTATTAGTCATCGCTATTACCATGGTGATGCGGT
TTTGGCAGTACATCAATGGGCGTGGATAGCGGTTTGACTCACGGGGATTTCCAAGTCTCCACCCCATTGACGTCAATGG
GAGTTTGTTTTGGCACCAAAATCAACGGGACTTTCCAAAATGTCGTAACAACTCCGCCCCATTGACGCAAATGGGCGGT
AGGCGTGTACGGTGGGAGGTCTATATAAGCAGAGCTCGTTTAGTGAACCGGGGTCTCTCTGGTTAGACCAGATCTGAGC
CTGGGAGCTCTCTGGCTAACTAGGGAACCCACTGCTTAAGCCTCAATAAAGCTTGCCTTGAGTGCTTCAAGTAGTGTGT
GCCCGTCTGTTGTGTGACTCTGGTAACTAGAGATCCCTCAGACCCTTTTAGTCAGTGTGGAAAATCTCTAGCAGTGGCGC
CCGAACAGGGACCTGAAAGCGAAAGGGAAACCAGAGGAGCTCTCTCGACGCAGGACTCGGCTTGCTGAAGCGCGCACGG
CAAGAGGCGAGGGGCGGCGACTGGTGAGTACGCCAAAAATTTTGACTAGCGGAGGCTAGAAGGAGAGAGATGGGTGCGA
GAGCGTCAGTATTAAGCGGGGGAGAATTAGATCGCGATGGGAAAAAATTCGGTTAAGGCCAGGGGGAAAGAAAAAATAT
AAATTAAAACATATAGTATGGGCAAGCAGGGAGCTAGAACGATTCGCAGTTAATCCTGGCCTGTTAGAAACATCAGAAG
GCTGTAGACAAATACTGGGACAGCTACAACCATCCCTTCAGACAGGATCAGAAGAACTTAGATCATTATATAATACAGTA
GCAACCCTCTATTGTGTGCATCAAAGGATAGAGATAAAAGACACCAAGGAAGCTTTAGACAAGATAGAGGAAGAGCAAAAC
AAAAGTAAGACCACCGCACAGCAAGCGGCCGCTGATCTTCAGACCTGGAGGAGGAGATATGAGGGACAATTGGAGAAGTGA
ATTATATAAATATAAAGTAGTAAAAATTGAACCATTAGGAGTAGCACCCACCAAGGCAAAGAGAAGAGTGGTGCAGAGAGA
AAAAAGAGCAGTGGGAATAGGAGCTTTGTTCCTTGGGTTCTTGGGAGCAGCAGGAAGCACTATGGGCGCAGCCTCAATGA
CGCTGACGGTACAGGCCAGACAATTATTGTCTGGTATAGTGCAGCAGCAGAACAATTTGCTGAGGGCTATTGAGGCGCA
ACAGCATCTGTTGCAACTCACAGTCTGGGGCATCAAGCAGCTCCAGGCAAGAATCCTGGCTGTGGAAAGATACCTAAAG
GATCAACAGCTCCTGGGGATTTGGGGTTGCTCTGGAAAACTCATTTGCACCACTGCTGTGCCTTGGAATGCTAGTTGGA
GTAATAAATCTCTGGAACAGATTGGAATCACACGACCTGGATGGAGTGGGACAGAGAAATTAACAATTACACAAGCTTA
ATACACTCCTTAATTGAAGAATCGCAAAACCAGCAAGAAAAGAATGAACAAGAATTATTGGAATTAGATAAATGGGCAA
GTTTGTGGAATTGGTTTAACATAACAAATTGGCTGTGGTATATAAAATTATTCATAATGATAGTAGGAGGCTTGGTAGGT
TTAAGAATAGTTTTTGCTGTACTTTCTATAGTGAATAGAGTTAGGCAGGGATATTCACCATTATCGTTTCAGACCCACCT
CCCAACCCCGAGGGGACCCGACAGGCCCGAAGGAATAGAAGAAGAAGGTGGAGAGAGAGACAGAGACAGATCCATTCGA
TTAGTGAACGGATCTCGACGGTATCGATAAGCTAATTCACAAATGGCAGTATTCATCCACAATTTTAAAAGAAAAGGGG
GGATTGGGGGGTACAGTGCAGGGGAAAGAATAGTAGACATAATAGCAACAGACATACAAACTAAAGAATTACAAAAACA
AATTACAAAAATTCAAAATTTTCGGGTTTATTACAGGGACAGCAGAGATCCAGTTTGGGAATTAGCTTGATCGATTAGTC
CAATTTGTTAAAGACAGGATATCAGTGGTCCAGGCTCTAGTTTTGACTCAACAATATCACCAGCTGAAGCCTATAGAGT
ACGAGCCATAGATAGAATAAAAGATTTTATTTAGTCTCCAGAAAAAGGGGGGAATGAAAGACCCCACCTGTAGGTTTGG
CAAGCTAGGATCAAGGTTAGGAACAGAGAGACAGCAGAATATGGGCCAAACAGGATATCTGTGGTAAGCAGTTCCTGCC
CCGGCTCAGGGCCAAGAACAGTTGGAACAGCAGAATATGGGCCAAACAGGATATCTGTGGTAAGCAGTTCCTGCCCCGG
CTCAGGGCCAAGAACAGATGGTCCCCAGATGCGGTCCCGCCCTCAGCAGTTTCTAGAGAACCATCAGATGTTTCCAGGG
TGCCCCAAGGACCTGAAATGACCCTGTGCCTTATTTGAACTAACCAATCAGTTCGCTTCTCGCTTCTGTTCGCGCGCTTC
TGCTCCCCGAGCTCAATAAAAGAGCCCACAACCCCTCACTCGGCGCGATCTAGATCTCGAATCGAATTCGAGCTCGGTA
CCCCCGCCCCCGCGTCTCCAGGGCAACCGTGGCTTTCGATTGTTACTGTGGGAACTGGAGGTAACAGTCTACAGCCATG
GTCGCCCCGCAGCACGCCCACGCGCGATATCGGGCCCGCGGTACCGTCGACTGCAGAATTCTACCGGGTAGGGGAGGCG
CTTTTCCCAAGGCAGTCTGGAGCATGCGCTTTAGCAGCCCCGCTGGCACTTGGCGCTACACAAGTGGCCTCTGGCCTCG
CACACATTCCACATCCACCGGTAGGCGCCAACCGGCTCCGTTCTTTGGTGGCCCCTTCGCGCCACCTTCTACTCCTCCC
CTAGTCAGGAAGTTCCCCCCCGCCCCGCAGCTCGCGTCGTGCAGGACGTGACAAATGGAAGTAGCACGTCTCACTAGTC
TCGTGCAGATGGACAGCACCGCTGAGCAATGGAAGCGGGTAGGCCTTTGGGGCAGCGGCCAATAGCAGCTTTGCTCCTT
CGCTTTCTGGGCTCAGAGGCTGGGAAGGGGTGGGTCCGGGGGCGGGCTCAGGGGCGGGCTCAGGGGCGGGGCGGGCGCC
CGAAGGTCCTCCGGAGGCCCGGCATTCTCGCACGCTTCAAAAGCGCACGTCTGCCGCGCTGTTCTCCTCTTCCTCATCT
CCGGGCCTTTCGACCATCTAGATCCACCGGTCGCCACCATGGTGAGCAAGGGCGAGGAGGTCATCAAAGAGTTCATGCG
CTTCAAGGTGCGCATGGAGGGCTCCATGAACGGCCACGAGTTCGAGATCGAGGGCGAGGGCGAGGGCCGCCCCTACGAG
GGCACCCAGACCGCCAAGCTGAAGGTGACCAAGGGCGGCCCCCTGCCCTTCGCCTGGGACATCCTGTCCCCCCAGTTCA
TGTACGGCTCCAAGGCGTACGTGAAGCACCCCGCCGACATCCCCGATTACAAGAAGCTGTCCTTCCCCGAGGGCTTCAA
GTGGGAGCGCGTGATGAACTTCGAGGACGGCGGTCTGGTGACCGTGACCCAGGACTCCTCCCTGCAGGACGGCACGCTG
ATCTACAAGGTGAAGATGCGCGGCACCAACTTCCCCCCCGACGGCCCCGTAATGCAGAAGAAGACCATGGGCTGGGAGG
CCTCCACCGAGCGCCTGTACCCCCGCGACGGCGTGCTGAAGGGCGAGATCCACCAGGCCCTGAAGCTGAAGGACGGCGG
CCACTACCTGGTGGAGTTCAAGACCATCTACATGGCCAAGAAGCCCGTGCAACTGCCCGGCTACTACTACGTGGACACC
AAGCTGGACATCACCTCCCACAACGAGGACTACACCATCGTGGAACAGTACGAGCGCTCCGAGGGCCGCCACCACCTGT
TCCTGGGGCATGGCACCGGCAGCACCGGCAGCGGCAGCTCCGGCACCGCCTCCTCCGAGGACAACAACATGGCCGTCAT
CAAAGAGTTCATGCGCTTCAAGGTGCGCATGGAGGGCTCCATGAACGGCCACGAGTTCGAGATCGAGGGCGAGGGCGAG
GGCCGCCCCTACGAGGGCACCCAGACCGCCAAGCTGAAGGTGACCAAGGGCGGCCCCCTGCCCTTCGCCTGGGACATCC
TGTCCCCCCAGTTCATGTACGGCTCCAAGGCGTACGTGAAGCACCCCGCCGACATCCCCGATTACAAGAAGCTGTCCTT
CCCCGAGGGCTTCAAGTGGGAGCGCGTGATGAACTTCGAGGACGGCGGTCTGGTGACCGTGACCCAGGACTCCTCCCTG
CAGGACGGCACGCTGATCTACAAGGTGAAGATGCGCGGCACCAACTTCCCCCCCGACGGCCCCGTAATGCAGAAGAAGA
CCATGGGCTGGGAGGCCTCCACCGAGCGCCTGTACCCCCGCGACGGCGTGCTGAAGGGCGAGATCCACCAGGCCCTGAA
GCTGAAGGACGGCGGCCACTACCTGGTGGAGTTCAAGACCATCTACATGGCCAAGAAGCCCGTGCAACTGCCCGGCTAC
TACTACGTGGACACCAAGCTGGACATCACCTCCCACAACGAGGACTACACCATCGTGGAACAGTACGAGCGCTCCGAGG
GCCGCCACCACCTGTTCCTGTACGGCATGGACGAGCTGTACAAGTAGGCGGCCGGGGTCGACTGATCCGATAATCAACC
TCTGGATTACAAAATTTGTGAAAGATTGACTGGTATTCTTAACTATGTTGCTCCTTTTACGCTATGTGGATACGCTGCTT
TAATGCCTTTGTATCATGCTATTGCTTCCCGTATGGCTTTCATTTTCTCCTCCTTGTATAAATCCTGGTTGCTGTCTCTT
TATGAGGAGTTGTGGCCCGTTGTCAGGCAACGTGGCGTGGTGTGCACTGTGTTTGCTGACGCAACCCCCACTGGTTGGG
GCATTGCCACCACCTGTCAGCTCCTTTCCGGGACTTTCGCTTTCCCCCTCCCTATTGCCACGGCGGAACTCATCGCCGC
CTGCCTTGCCCGCTGCTGGACAGGGGCTCGGCTGTTGGGCACTGACAATTCCGTGGTGTTGTCGGGGAAATCATCGTCC
TTTCCTTGGCTGCTCGCCTGTGTTGCCACCTGGATTCTGCGCGGGACGTCCTTCTGCTACGTCCCTTCGGCCCTCAATCC
AGCGGACCTTCCTTCCCGCGGCCTGCTGCCGGCTCTGCGGCCTCTTCCGCGTCTTCGCCTTCGCCCTCAGACGAGTCGG
ATCTCCCTTTGGGCCGCCTCCCCGCATCGGATCAAATTCGAGCTCGGTACCTTTAAGACCAATGACTTACAAGGCAGCT
GTAGATCTTAGCCACTTTTTAAAAGAAAAGGGGGGACTGGAAGGGCTAATTCACTCCCAACGAAGACAAGATCTGCTTT
TTGCTTGTACTGGGTCTCTCTGGTTAGACCAGATCTGAGCCTGGGAGCTCTCTGGCTAACTAGGGAACCCACTGCTTAA
GCCTCAATAAAGCTTGCCTTGAGTGCTTCAAGTAGTGTGTGCCCGTCTGTTGTGTGACTCTGGTAACTAGAGATCCCTCA
GACCCTTTTAGTCAGTGTGGAAAATCTCTAGCAGTAGTAGTTCATGTCATCTTATTATTCAGTATTTATAACTTGCAAAG
AAATGAATATCAGAGAGTGAGAGGAACTTGTTTATTGCAGCTTATAATGGTTACAAATAAAGCAATAGCATCACAAATTT
CACAAATAAAGCATTTTTTTCACTGCATTCTAGTTGTGGTTTGTCCAAACTCATCAATGTATCTTATCATGTCTGGCTCT
AGCTATCCCGCCCCTAACTCCGCCCATCCCGCCCCTAACTCCGCCCAGTTCCGCCCATTCTCCGCCCCATGGCTGACTA
ATTTTTTTTATTTATGCAGAGGCCGAGGCCGCCTCGGCCTCTGAGCTATTCCAGAAGTAGTGAGGAGGCTTTTTTGGAGG
CCTAGGCTTTTGCGTCGAGACGTACCCAATTCGCCCTATAGTGAGTCGTATTACGCGCGCTCACTGGCCGTCGTTTTAC
AACGTCGTGACTGGGAAAACCCTGGCGTTACCCAACTTAATCGCCTTGCAGCACATCCCCCTTTCGCCAGCTGGCGTAA
TAGCGAAGAGGCCCGCACCGATCGCCCTTCCCAACAGTTGCGCAGCCTGAATGGCGAATGGCGCGACGCGCCCTGTAGC
GGCGCATTAAGCGCGGCGGGTGTGGTGGTTACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAAGCTCTAAATCGGGGGCTCCCTTTAGGGTTC
CGATTTAGTGCTTTACGGCACCTCGACCCCAAAAAACTTGATTAGGGTGATGGTTCACGTAGTGGGCCATCGCCCTGAT
AGACGGTTTTTCGCCCTTTGACGTTGGAGTCCACGTTCTTTAATAGTGGACTCTTGTTCCAAACTGGAACAACACTCAAC
CCTATCTCGGTCTATTCTTTTGATTTATAAGGGATTTTGCCGATTTCGGCCTATTGGTTAAAAAATGAGCTGATTTAACA
AAAATTTAACGCGAATTTTAACAAAATATTAACGTTTACAATTTCC
Sequence ID No.: 19—165A VEGF isoform
GAATTCG
CCCTTCCTGA GATCACCGGT AGGAGGGCCA TCATGAACTT TCTGCTGTCT TGGGTGCATT
GGAGCCTTGC CTTGCTGCTC TACCTCCACC ATGCCAAGTG GTCCCAGGCT GCACCCATGG
CAGAAGGAGG AGGGCAGAAT CATCACGAAG TGGTGAAGTT CATGGATGTC TATCAGCGCA
GCTACTGCCA TCCAATCGAG ACCCTGGTGG ACATCTTCCA GGAGTACCCT GATGAGATCG
AGTACATCTT CAAGCCATCC TGTGTGCCCC TGATGCGATG CGGGGGCTGC TGCAATGACG
AGGGCCTGGA GTGTGTGCCC ACTGAGGAGT CCAACATCAC CATGCAGATT ATGCGGATCA
AACCTCACCA AGGCCAGCAC ATAGGAGAGA TGAGCTTCCT ACAGCACAAC AAATGTGAAT
GCAGACCAAA GAAAGATAGA GCAAGACAAG AAAATCCCTG TGGGCCTTGC TCAGAGCGGA
GAAAGCATTT GTTTGTACAA GATCCGCAGA CGTGTAAATG TTCCTGCAAA AACACAGACT
CGCGTTGCAA GGCGAGGCAG CTTGAGTTAA ACGAACGTAC TTGCAGATGT GACAAGCCGA
GGCGGTGAAA GGGCGAATTC

1. A highly purified population of cell-derived vesicles prepared by culturing stem cells producing the cell-derived vesicles under conditions of hypoxia and low serum conditions, optionally wherein the cell-derived vesicles comprise exosomes and/or microvesicles.

2. (canceled)

3. The purified population of claim 1, wherein the cell-derived vesicles are isolated from one or more stem cells of the group of adult stem cells, embryonic stem cells, embryonic-like stem cells, neural stem cells, mesenchymal stem cells, or induced pluripotent stem cells.

4. (canceled)

5. The purified population of claim 1, wherein the cell-derived vesicles of the population further comprise at least one exogenous nucleic acid and/or at least one exogenous protein, optionally wherein the population of cell-derived vesicles do not comprise exogenous VEGFR and/or VEGF.

6. The purified population of claim 5, wherein the exogenous nucleic acid encodes a micro RNA (miRNA), optionally wherein the miRNA is selected from the group consisting of miR-150, miR-126, miR-132, miR-296, and let-7.

7. (canceled)

8. The purified population of claim 5, wherein the exogenous protein is one or more of platelet derived growth factor receptor (PDGFR), Collagen, Type 1, Alpha 2 (COL1A2), Collagen, Type VI, Alpha 3 (COL6A3), EGF-like repeats- and discoidin i-like domains-containing protein 3 (EDIL3), epidermal growth factor receptor (EGFR), fibroblast growth factor receptor (FGFR), fibronectin (FN1), Milk fat globule-EGF factor 8 (MFGE8), lectin, galactoside-binding, soluble, 3 binding protein (LGALS3BP), nuclear factor-kappaB (NFκB), or transferrin (TF).

9. (canceled)

10. The purified population of claim 1, wherein the cell-derived vesicles of the population comprise one or more of miR-126, miR-132, miR-150, miR-210, miR-214, miR-296, and miR-424.

11-16. (canceled)

17. The purified population of claim 1, wherein the cell-derived vesicles of the population comprise one or more of 3,6-anhydro-D-galactose, 4-aminobutyric acid, 5′-deoxy-5′-methylthioadenosine, 5-methoxytryptamine, s-adenosylmethionine, s-adenosylhomocysteine, adipic acid, aminomalonate, arabinose, aspartic acid, beta-alanine, cholesterol, citric acid, creatinine, cysteine, cytidine-5-monophosphate, erythritol, fructose, fumaric acid, galacturonic acid, glucose, glucose-1-phosphate, glucose-6-phosphate, glutamine, glyceric acid, glycerol-alpha-phosphate, glycine, guanosine, hexitol, hexuronic acid, inosine, isohexonic acid, isomaltose, lactamide, lactic acid, lactose, leucine, levoglucosan, maleimide, malic acid, maltotriose, mannose, methanolphosphate, methionine, N-acetylaspartic acid, N-acetyl-D-galactosamine, nicotinamide, N-methylalanine, oxoproline, pantothenic acid, pentadecanoic acid, phenol, putrescine, pyruvic acid, ribitol, ribose, sorbitol, squalene, succinic acid, threitol, threonic acid, threonine, thymine, trans-4-hydroxyproline, trehalose, urea, uridine, valine, and xylitol.

18-26. (canceled)

27. The purified population of claim 1, wherein the cell-derived vesicles of the population comprise one or more of Ceramide (d32:1), Ceramide (d33:1), Ceramide (d34:0), Ceramide (d34:1), Ceramide (d34:2), Ceramide (d34:2), Ceramide (d36:1), Ceramide (d38:1), Ceramide (d39:1), Ceramide (d40:0), Ceramide (d40:1), Ceramide (d40:2), Ceramide (d41:1), Ceramide (d42:1), Ceramide (d42:2) B, Ceramide (d44:1), Fatty Acid (20:4), Fatty Acid (22:0), Fatty Acid (22:6), Fatty Acid (24:0), Fatty Acid (24:1), glucosylceramides (d40:1), glucosylceramides (d41:1), glucosylceramides (d42:1), glucosylceramides (d42:2), Lysophosphatidylcholines (16:0), Lysophosphatidylcholines (18:0) A, Lysophosphatidylcholines (18:1), lysophosphatidylethanolamine (20:4), Phosphatidylcholines (32:1), Phosphatidylcholines (33:1), Phosphatidylcholines (34:0), Phosphatidylcholines (34:1), Phosphatidylcholines (34:2), Phosphatidylcholines (35:2), Phosphatidylcholines (36:1), Phosphatidylcholines (36:2), Phosphatidylcholines (36:3), Phosphatidylcholines (38:2), Phosphatidylcholines (38:3), Phosphatidylcholines (38:5), Phosphatidylcholines (38:6), Phosphatidylcholines (40:5), Phosphatidylcholines (40:6), Phosphatidylcholines (40:7), Phosphatidylcholines (p-34:0), Phosphatidylcholines (o-34:1), Phosphatidylethanolamines (34:1), Phosphatidylethanolamines (34:2), Phosphatidylethanolamines (36:3), Phosphatidylethanolamines (36:4), Phosphatidylethanolamines (38:4), B Phosphatidylethanolamines (38:6), Phosphatidylethanolamines (p-34:1), Phosphatidylethanolamines (o-34:2), Phosphatidylethanolamines (p-36:1), Phosphatidylethanolamines (o-36:2), Phosphatidylethanolamines (p-36:4), Phosphatidylethanolamines (o-36:5), Phosphatidylethanolamines (p-38:4), Phosphatidylethanolamines (o-38:5), Phosphatidylethanolamines (p-38:5), Phosphatidylethanolamines (o-38:6), Phosphatidylethanolamines (p-38:6), Phosphatidylethanolamines (o-38:7), Phosphatidylethanolamines (p-40:4), Phosphatidylethanolamines (o-40:5), Phosphatidylethanolamines (p-40:5), Phosphatidylethanolamines (o-40:6), Phosphatidylethanolamines (p-40:6), Phosphatidylethanolamines (o-40:7), Phosphatidylethanolamines (p-40:7), Phosphatidylethanolamines (o-40:8), Sphingomyelin (d30:1), Sphingomyelin (d32:0), Sphingomyelin (d32:2), Sphingomyelin (d33:1), Sphingomyelin (d34:0), Sphingomyelin (d36:1), Sphingomyelin (d36:2), Sphingomyelin (d38:1), Sphingomyelin (d40:1), Sphingomyelin (d40:2), Sphingomyelin (d41:1), Sphingomyelin (d41:2), Sphingomyelin (d42:2), and B Sphingomyelin (d42:3).

28-36. (canceled)

37. The purified population of claim 1, wherein the cell-derived vesicles of the population comprise one or more of CD9, HSPA8, PDCD6IP, GAPDH, ACTB, ANXA2, CD63, SDCBP, ENO1, HSP90AA1, TSG101, PKM, LDHA, EEF1A1, YWHAZ, PGK1, EEF2, ALDOA, ANXA5, FASN, YWHAE, CLTC, CD81, ALB, VCP, TPI1, PPIA, MSN, CFL1, PRDX1, PFN1, RAP1B, ITGB1, HSPA5, SLC3A2, GNB2, ATP1A1, WHAQ, FLOT1, FLNA, CLIC1, CDC42, CCT2, A2M, YWHAG, RAC1, LGALS3BP, HSPA1A, GNAI2, ANXA1, RHOA, MFGE8, PRDX2, GDI2, EHD4, ACTN4, YWHAB, RAB7A, LDHB, GNAS, TFRC, RAB5C, ANXA6, ANXA11, KPNB1, EZR, ANXA4, ACLY, TUBA1C, RAB14, HIST2H4A, GNB1, UBA1, THBS1, RAN, RAB5A, PTGFRN, CCT5, CCT3, BSG, RAB5B, RAB1A, LAMP2, ITGA6, GSN, FN1, YWHAH, TKT, TCP1, STOM, SLC16A1, and RAB8A proteins.

38-46. (canceled)

47. The purified population of claim 1, wherein the cell-derived vesicles of the population comprise one or more of FN1, EDIL3, TF, ITGB1, VCAN, ANXA2, MFGE8, TGB1, TGFB2, TGFBR1, TGBFR2, TGFBI, TGFBRAP1, BASP1, COL1, COL6, GAPDH, ITGA3, FBN1, ITGAV, ITGB5, NOTCH2, SDCBP, HSPA2, HSPA8, NT5E, MRGPRF, RTN4, NEFM, INA, NRP1, HSPA9, FBN1, BSG, PRPH, FBLN1, PARP4, FLNA, YBX1, EVA1B, ADAM10, HSPG2, MCAM, POSTN, GNB2, GNB1, ANPEP, ADAM9, ATP1A1, CSPG4, EHD2, PXDN, SERPINE2, CAV1, PKM, GNB4, NPTN, CCT2, LGALS3BP, and MVP proteins.

48-56. (canceled)

57. The purified population of claim 1, wherein the cell-derived vesicles of the population comprise one or more of FBLN2, TIMP1, NID1, IGFBP3, LTBP1, DUSP3, ITGAV, LAMA5, COL1A1, NOTCH2, NRG1, ERBB2, COL4A2, LDLR, TSB, MMP2, TIMP2, TPI1, ACVR1B, INHBA, EGFR, APH1A, NCSTN, TGFB2, SPARC, TGFB1, F2, SERPINE1, SDC4, SDC3, ACAN, IFI16, MMP14, PLAT, COL18A1, NOTCH3, DSP, PKP4, SERPINE2, SRGN, NRP2, EPHA2, ITGA5, NRP1, PLAU, SERPINB6, CLEC3B, CD47, SDC1, PSMA7, ENG, S100A13, TIMP3, TMED10, TGFBI, CTGF, DCN, ITGB3, PDGFRA, JAG1, TGFBR2, PLAUR, PDGFRB, FYN, THY1, HSPG2, TENC1, TGFBR1, PLXNA1, LRP1, STAT1, CXCL12, VCAN, MET, FN1, CD36, STAT3, THBS1, FGFR1, GRB14, FGB, API5, HAPLN1, RECK, LAMC1, CYR61, GPC1, IGFBP4, ITGA4, MFAP2, SDC2, EFNB2, FGA, PLXND1, ADAM17, ADAM9, ANPEP, EPHB1, PPP2R5D, ANTXR2, IGFBP7, COL6A3, LAMB3, ADAMTS1, ADAM10, A2M, EFNB1, ITGA3, CLU, KHSRP, and EFEMP1 proteins associated with angiogenesis.

58-66. (canceled)

67. The purified population of claim 1, wherein the cell-derived vesicles of the population comprise one or more of TGFBI, TGFB1, TGFBR2, TGFBR1, TGFB2, TGFBRAP1, ADAM17, ARG1, CD274, EIF2A, EPHB2, HLA-DRA, ELAVL1, IRAK1, LGALS1, PSME4, STAT1, and STAT3 proteins associated with immune modulation.

68-76. (canceled)

77. The purified population of claim 1, wherein the cell-derived vesicles of the population comprise one or more of EDIL3, TF, ITGB1, ANXA2, MFGE8, TGB1, TGBFR2, BASP1, COL1, COL6, GAPDH, FBN1, ITGB5, SDCBP, HSPA2, HSPA8, NT5E, MRGPRF, RTN4, NEFM, INA, HSPA9, FBN1, BSG, PRPH, FBLN1, PARP4, FLNA, YBX1, EVA1B, MCAM, POSTN, GNB2, GNB1, ATP1A1, CSPG4, EHD2, PXDN, CAV1, PKM, GNB4, NPTN, CCT2, LGALS3BP, and MVP therapeutic proteins.

78-88. (canceled)

89. The purified population of claim 1, wherein the concentration of cell-derived vesicles in the population comprises (a) between about 0.5 micrograms and 5000 micrograms, (b) less than about 300 micrograms, or (c) less than about 200 micrograms of exosome and/or microvesicle protein collected per approximately 10⁶ cells.

90-91. (canceled)

92. The purified population of claim 1, wherein the average diameter of the cell-derived vesicles in the population is between about (a) 0.1 nm and about 1000 nm, (b) less than about 100 nm, (c) less than about 50 nm, or (d) less than about 40 nm.

93-95. (canceled)

96. The purified population of claim 1, wherein the cell-derived vesicles have been purified from by a method comprising filtration, optionally tangential flow filtration.

97. A composition comprising the purified population of cell-derived vesicles of claim 1 and a carrier, optionally wherein the carrier is a pharmaceutically acceptable carrier, and optionally an additional therapeutic agent.

98. (canceled)

99. The composition of claim 97, further comprising an isolated stem cell, optionally wherein the isolated stem cell is selected from the group of an adult stem cell, an embryonic stem cell, an induced pluripotent stem cell, an embryonic-like stem cell, a mesenchymal stem cell, or a neural stem cell.

100. (canceled)

101. A method for promoting angiogenesis, treating peripheral arterial disease or stroke, or treating a dermal wound in a subject in need thereof comprising administering to the subject the purified population claim 1, optionally wherein the subject is administered at least one dose of between approximately 0.1 mg and 200 mg of cell-derived vesicle protein.

102-122. (canceled)

123. A method for purifying a population of cell-derived vesicles, comprising:
(a) applying a tangential flow filtration to conditioned media produced by a population of isolated stem cells to isolate a cell-derived vesicles containing fraction; and
(b) concentrating the cell-derived vesicle containing fraction to provide a purified population of cell-derived vesicles,
optionally wherein after step (a) cell debris and other contaminates are removed from the cell-derived vesicle containing fraction prior to step (b),
and optionally wherein the isolated stem cells are one or more of adult stem cells, embryonic stem cells, embryonic-like stem cells, neural stem cells, mesenchymal stem cells, or induced pluripotent stem cells.

124. (canceled)

125. The method of claim 123, wherein the population of stem cells were cultured under hypoxic and low serum conditions for up to about 72 hours prior to performing step (a), optionally wherein the hypoxic conditions are between approximately 1%-15% CO₂ and between 0.05%-20% oxygen tension, and optionally wherein the low serum conditions are serum free conditions.

126-133. (canceled)

134. The method of claim 123, wherein step (b) is performed using a filtration device, optionally wherein the filtration device is a 100 or 300 kilodalton nominal molecular weight limit filtration device.

135-136. (canceled)

137. The method of claim 123, further comprising formulating the purified population of cell-derived vesicles by mixing the population with a carrier and/or a stabilizer and drying, freezing or freeze drying the purified population of cell-derived vesicles.

138-139. (canceled)

140. A dried, lyophilized or frozen population of cell-derived vesicles of the purified population of claim 1.

141. A kit comprising the population of claim 140 and instructions for use.

142. A method for large-scale purification of a population of cell-derived vesicles, comprising:
(a) applying a tangential flow filtration to conditioned media produced by a population of isolated stem cells cultured in a bioreactor to isolate a cell-derived vesicles containing fraction, optionally wherein the bioreactor is a hollow fiber bioreactor; and
(b) concentrating the cell-derived vesicle containing fraction to provide a purified population of cell-derived vesicles.

143. (canceled)


SEQUENCE LISTING (19 sequences)

SEQ ID NO: 1 - DNA, 84 bp, Homo sapiens
ctccccatggccctgtctcccaacccttgtaccagtgctgggctcagaccctggtacaggcctgggggacagggacctggggac

SEQ ID NO: 2 - DNA, 85 bp, Homo sapiens
cgctggcgacgggacattattacttttggtacgcgctgtgacacttcaaactcgtaccgtgagtaataatgcgccgtccacggca

SEQ ID NO: 3 - DNA, 80 bp, Homo sapiens
aggacccttccagagggccccccctcaatcctgttgtgcctaattcagagggttgggtggaggctctcctgaagggctct

SEQ ID NO: 4 - DNA, 80 bp, Homo sapiens
tgggatgaggtagtaggttgtatagttttagggtcacacccaccactgggagataactatacaatctactgtctttccta

SEQ ID NO: 5 - DNA, 6574 bp, Homo sapiens
aagagcaaaaagcgaaggcgcaatctggacactgggagattcggagcgcagggagtttgagagaaacttttattttgaagagaccaaggttgagggggggcttatttcctgacagctatttacttagagcaaatgattagttttagaaggatggactataacattgaatcaattacaaaacgcggtttttgagcccattactgttggagctacagggagagaaacagaggaggagactgcaagagatcattggaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggatgacatcggcatagactcttcagacctggtggaagacagcttcctgtaactggcggattcgaggggttccttccacttctggggccacctctggatcccgttcagaaaaccactttattgcaatgcagaggttgagaggaggacttggttgatgtttaaagagaagttcccagccaagggcctcggggagcgttctaaatatgaatgaatgggatattttgaaatgaactttgtcagtgttgcctcttgcaatgcctcagtagcatctcagtggtgtgtgaagtttggagatagatggataagggaataataggccacagaaggtgaactttgtgcttcaaggacattggtgagagtccaacagacacaatttatactgcgacagaacttcagcattgtaattatgtaaataactctaaccaaggctgtgtttagattgtattaactatcttctttggacttctgaagagaccactcaatccatccatgtacttccctcttgaaacctgatgtcagctgctgttgaactttttaaagaagtgcatgaaaaaccatttttgaaccttaaaaggtactggtactatagcattttgctatcttttttagtgttaaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccagagaaagcacaatttaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcaaaaagactggatttgcagaagttttttttttttttttcttcatgcctgatgaaagctttggcgaccccaatatatgtattttttgaatctatgaacctgaaaagggtcagaaggatgcccagacatcagcctccttctttcaccccttaccccaaagagaaagagtttgaaactcgagaccataaagatattctttagtggaggctggatgtgcattagcctggatcctcagttctcaaatgtgtgtggcagccaggatgactagatcctgggtttccatccttgagattctgaagtatgaagtctgagggaaaccagagtctgtatttttctaaactccctggctgttctgatcggccagttttcggaaacactgacttaggtttcaggaagttgccatgggaaacaaataatttgaactttggaacagggttggcattcaaccacgcaggaagcctactatttaaatccttggcttcaggttagtgacatttaatgccatctagctagcaattgcgaccttaatttaactttccagtcttagctgaggctgagaaagctaaagtttggttttgacaggttttccaaaagtaaagatgctacttcccactgtatgggggagattgaactttccccgtctcccgtcttctgcctcccactccataccccgccaaggaaaggcatgtacaaaaattatgcaattcagtgttccaagtctctgtgtaaccagctcagtgttttggtggaaaaaacattttaagttttactgataatttgaggttagatgggaggatgaattgtcacatctatccacactgtcaaacaggttggtgtgggttcattggcattctttgcaatactgcttaattgctgataccatatgaatgaaacatgggctgtgattactgcaatcactgtgctatcggcagatgatgctttggaagatgcagaagcaataataaagtacttgactacctactggtgtaatctcaatgcaagccccaactttcttatccaactttttcatagtaagtgcgaagactgagccagattggccaattaaaaacgaaaacctgactaggttctgtagagccaattagacttgaaatacgtttgtgtttctagaatcacagctcaagcattctgtttatcgctcactctcccttgtacagccttattttgttggtgctttgcattttgatattgctgtgagccttgcatgacatcatgaggccggatgaaacttctcagtccagcagtttccagtcctaacaaatgctcccacctgaatttgtatatgactgcatttgtgtgtgtgtgtgtgttttcagcaaattccagatttgtttccttttggcctcctgcaaagtctccagaagaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtttttgttttaaaacgtgtaaatgaagatctttatatttcaataaatgatatataatttaaagtta

SEQ ID NO: 6 - DNA, 5718 bp, Homo sapiens
ctcctgaggctgccagcagccagcagtgactgcccgccctatctgggacccaggatcgctctgtgagcaacttggagccagagaggagatcaacaaggaggaggagagagccggcccctcagccctgctgcccagcagcagcctgtgctcgccctgcccaacgcagacagccagacccagggcggcccctctggcggctctgctcctcccgaaggatgcttggggagtgaggcgaagctgggccgctcctctcccctacagcagcccccttcctccatccctctgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggacaccatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtagggggctggcccctaccctgccctgcctgaagctccccccctgccagcacccagcatctcctggcctggcctgaccgggcttcctgtcagccaggctgcccttatcagctgtccccttctggaagctttctgctcctgacgtgttgtgccccaaaccctggggctggcttaggaggcaagaaaactgcaggggccgtgaccagccctctgcctccagggaggccaactgactctgagccagggttcccccagggaactcagttttcccatatgtaagatgggaaagttaggcttgatgacccagaatctaggattctctccctggctgacaggtggggagaccgaatccctccctgggaagattcttggagttactgaggtggtaaattaacttttttctgttcagccagctacccctcaaggaatcatagctctctcctcgcacttttatccacccaggagctagggaagagaccctagcctccctggctgctggctgagctagggcctagccttgagcagtgttgcctcatccagaagaaagccagtctcctccctatgatgccagtccctgcgttccctggcccgagctggtctggggccattaggcagcctaattaatgctggaggctgagccaagtacaggacacccccagcctgcagcccttgcccagggcacttggagcacacgcagccatagcaagtgcctgtgtccctgtccttcaggcccatcagtcctggggctttttctttatcaccctcagtcttaatccatccaccagagtctagaaggccagacgggccccgcatctgtgatgagaatgtaaatgtgccagtgtggagtggccacgtgtgtgtgccagtatatggccctggctctgcattggacctgctatgaggctttggaggaatccctcaccctctctgggcctcagtttccccttcaaaaaatgaataagtcggacttattaactctgagtgccttgccagcactaacattctagagtattccaggtggttgcacatttgtccagatgaagcaaggccatataccctaaacttccatcctgggggtcagctgggctcctgggagattccagatcacacatcacactctggggactcaggaaccatgccccttccccaggcccccagcaagtctcaagaacacagctgcacaggccttgacttagagtgacagccggtgtcctggaaagcccccagcagctgccccagggacatgggaagaccacgggacctctttcactacccacgatgacctccgggggtatcctgggcaaaagggacaaagagggcaaatgagatcacctcctgcagcccaccactccagcacctgtgccgaggtctgcgtcgaagacagaatggacagtgaggacagttatgtcttgtaaaagacaagaagcttcagatgggtaccccaagaaggatgtgagaggtgggcgctttggaggtttgcccctcacccaccagctgccccatccctgaggcagcgctccatgggggtatggttttgtcactgcccagacctagcagtgacatctcattgtccccagcccagtgggcattggaggtgccaggggagtcagggttgtagccaagacgcccccgcacggggagggttgggaagggggtgcaggaagctcaacccctctgggcaccaaccctgcattgcaggttggcaccttacttccctgggatccccagagttggtccaaggagggagagtgggttctcaatacggtaccaaagatataatcacctaggtttacaaatatttttaggactcacgttaactcacatttatacagcagaaatgctattttgtatgctgttaagtttttctatctgtgtacttttttttaagggaaagattttaatattaaacctggtgcttctcactcacaaaaa

SEQ ID NO: 7 - skipped

SEQ ID NO: 8 - DNA, 10599 bp, Homo sapiens
aagccctgactggtatccctggccccagtccagtttggagctcagtcttccaccaaaggccgttcagttctcctgggctccagcctcctgcaaggactgcaagagttttcctccgcagctctgagtctccacttttttggtggagaaaggctgcaaaaagaaaaagagacgcagtgagtgggaaaagtatgcatcctattcaaacctaattgaatcgaggagcccagggacacacgccttcaggtttgctcaggggttcatatttggtgcttagacaaattcaaaatgaggaaacatcggcacttgcccttagtggccgtcttttgcctctttctctcaggctttcctacaactcatgcccagcagcagcaagcagatgtcaaaaatggtgcggctgctgatataatatttctagtggattcctcttggaccattggagaggaacatttccaacttgttcgagagtttctatatgatgttgtaaaatccttagctgtgggagaaaatgatttccattttgctctggtccagttcaacggaaacccacataccgagttcctgttaaatacgtatcgtactaaacaagaagtcctttctcatatttccaacatgtcttatattgggggaaccaatcagactggaaaaggattagaatacataatgcaaagccacctcaccaaggctgctggaagccgggccggtgacggagtccctcaggttatcgtagtgttaactgatggacactcgaaggatggccttgctctgccctcagcggaacttaagtctgctgatgttaacgtgtttgcaattggagttgaggatgcagatgaaggagcgttaaaagaaatagcaagtgaaccgctcaatatgcatatgttcaacctagagaattttacctcacttcatgacatagtaggaaacttagtgtcctgtgtgcattcatccgtgagtccagaaagggctggggacacggaaacccttaaagacatcacagcacaagactctgctgacattattttccttattgatggatcaaacaacaccggaagtgtcaatttcgcagtcattctcgacttccttgtaaatctccttgagaaactcccaattggaactcagcagatccgagtgggggtggtccagtttagcgatgagcccagaaccatgttctccttggacacctactccaccaaggcccaggttctgggtgcagtgaaagccctcgggtttgctggtggggagttggccaatatcggcctcgcccttgatttcgtggtggagaaccacttcacccgggcagggggcagccgcgtggaggaaggggttccccaggtgctggtcctcataagtgccgggccttctagtgacgagattcgctacggggtggtagcactgaagcaggctagcgtgttctcattcggccttggagcccaggccgcctccagggcagagcttcagcacatagctaccgatgacaacttggtgtttactgtcccggaattccgtagctttggggacctccaggagaaattactgccgtacattgttggcgtggcccaaaggcacattgtcttgaaaccgccaaccattgtcacacaagtcattgaagtcaacaagagagacatagtcttcctggtggatggctcatctgcactgggactggccaacttcaatgccatccgagacttcattgctaaagtcatccagaggctggaaatcggacaggatcttatccaggtggcagtggcccagtatgcagacactgtgaggcctgaattttatttcaatacccatccaacaaaaagggaagtcataaccgctgtgcggaaaatgaagcccctggacggctcggccctgtacacgggctctgctctagactttgttcgtaacaacctattcacgagttcagccggctaccgggctgccgaggggattcctaagcttttggtgctgatcacaggtggtaagtccctagatgaaatcagccagcctgcccaggagctgaagagaagcagcataatggcctttgccattgggaacaagggtgccgatcaggctgagctggaagagatcgctttcgactcctccctggtgttcatcccagctgagttccgagccgccccattgcaaggcatgctgcctggcttgctggcacctctcaggaccctctctggaacccctgaagttcactcaaacaaaagggatatcatctttcttttggatggatcagccaacgttggaaaaaccaatttcccttatgtgcgcgactttgtaatgaacctagttaacagccttgatattggaaatgacaatattcgtgttggtttagtgcaatttagtgacactcctgtaacggagttctctttaaacacataccagaccaagtcagatatccttggtcatctgaggcagctgcagctccagggaggttcgggcctgaacacaggctcagccctaagctatgtctatgccaaccacttcacggaagctggcggcagcaggatccgtgaacacgtgccgcagctcctgcttctgctcacagctgggcagtctgaggactcctatttgcaagctgccaacgccttgacacgcgcgggcatcctgactttttgtgtgggagctagccaggcgaataaggcagagcttgagcagattgcttttaacccaagcctggtgtatctcatggatgatttcagctccctgccagctttgcctcagcagctgattcagcccctaaccacatatgttagtggaggtgtggaggaagtaccactcgctcagccagagagcaagcgagacattctgttcctctttgacggctcagccaatcttgtgggccagttccctgttgtccgtgactttctctacaagattatcgatgagctcaatgtgaagccagaggggacccgaattgcggtggctcagtacagcgatgatgtcaaggtggagtcccgttttgatgagcaccagagtaagcctgagatcctgaatcttgtgaagagaatgaagatcaagacgggcaaagccctcaacctgggctacgcgctggactatgcacagaggtacatttttgtgaagtctgctggcagccggatcgaggatggagtgcttcagttcctggtgctgctggtcgcaggaaggtcatctgaccgtgtggatgggccagcaagtaacctgaagcagagtggggttgtgcctttcatcttccaagccaagaacgcagaccctgctgagttagagcagatcgtgctgtctccagcgtttatcctggctgcagagtcgcttcccaagattggagatcttcatccacagatagtgaatctcttaaaatcagtgcacaacggagcaccagcaccagtttcaggtgaaaaggacgtggtgtttctgcttgatggctctgagggcgtcaggagcggcttccctctgttgaaagagtttgtccagagagtggtggaaagcctggatgtgggccaggaccgggtccgcgtggccgtggtgcagtacagcgaccggaccaggcccgagttctacctgaattcatacatgaacaagcaggacgtcgtcaacgctgtccgccagctgaccctgctgggagggccgacccccaacaccggggccgccctggagtttgtcctgaggaacatcctggtcagctctgcgggaagcaggataacagaaggtgtgccccagctgctgatcgtcctcacggccgacaggtctggggatgatgtgcggaacccctccgtggtcgtgaagaggggtggggctgtgcccattggcattggcatcgggaacgctgacatcacagagatgcagaccatctccttcatcccggactttgccgtggccattcccacctttcgccagctggggaccgtccaacaggtcatctctgagagggtgacccagctcacccgcgaggagctgagcaggctgcagccggtgttgcagcctctaccgagcccaggtgttggtggcaagagggacgtggtctttctcatcgatgggtcccaaagtgccgggcctgagttccagtacgttcgcaccctcatagagaggctggttgactacctggacgtgggctttgacaccacccgggtggctgtcatccagttcagcgatgaccccaaggtggagttcctgctgaacgcccattccagcaaggatgaagtgcagaacgcggtgcagcggctgaggcccaagggagggcggcagatcaacgtgggcaatgccctggagtacgtgtccaggaacatcttcaagaggcccctggggagccgcattgaagagggcgtcccgcagttcctggtcctcatctcgtctggaaagtctgacgatgaggtggacgacccggcggtggagctcaagcagtttggcgtggcccctttcacgatcgccaggaacgcagaccaggaggagctggtgaagatctcgctgagccccgaatatgtgttctcggtgagcaccttccgggagctgcccagcctggagcagaaactgctgacgcccatcacgaccctgacctcagagcagatccagaagctcttagccagcactcgctatccacctccagcagttgagagtgatgctgcagacattgtctttctgatcgacagctctgagggagttaggccagatggctttgcacatattcgagattttgttagcaggattgttcgaagactcaacatcggccccagtaaagtgagagttggggtcgtgcagttcagcaatgatgtcttcccagaattctatctgaaaacctacagatcccaggccccggtgctggacgccatacggcgcctgaggctcagaggggggtccccactgaacactggcaaggctctcgaatttgtggcaagaaacctctttgttaagtctgcggggagtcgcatagaagacggggtgccccaacacctggtcctggtcctgggtggaaaatcccaggacgatgtgtccaggttcgcccaggtgatccgttcctcgggcattgtgagtttaggggtaggagaccggaacatcgacagaacagagctgcagaccatcaccaatgaccccagactggtcttcacagtgcgagagttcagagagcttcccaacatagaagaaagaatcatgaactcgtttggaccctccgcagccactcctgcacctccaggggtggacacccctcctccttcacggccagagaagaagaaagcagacattgtgttcctgttggatggttccatcaacttcaggagggacagtttccaggaagtgcttcgttttgtgtctgaaatagtggacacagtttatgaagatggcgactccatccaagtggggcttgtccagtacaactctgaccccactgacgaattcttcctgaaggacttctctaccaagaggcagattattgacgccatcaacaaagtggtctacaaagggggaagacacgccaacactaaggtgggccttgagcacctgcgggtaaaccactttgtgcctgaggcaggcagccgcctggaccagcgggtccctcagattgcctttgtgatcacgggaggaaagtcggtggaagatgcacaggatgtgagcctggccctcacccagaggggggtcaaagtgtttgctgttggagtgaggaatatcgactcggaggaggttggaaagatagcgtccaacagcgccacagcgttccgcgtgggcaacgtccaggagctgtccgaactgagcgagcaagttttggaaactttgcatgatgcgatgcatgaaaccctttgccctggtgtaactgatgctgccaaagcttgtaatctggatgtgattctggggtttgatggttctagagaccagaatgtttttgtggcccagaagggcttcgagtccaaggtggacgccatcttgaacagaatcagccagatgcacagggtcagctgcagcggtggccgctcgcccaccgtgcgtgtgtcagtggtggccaacacgccctcgggcccggtggaggcctttgactttgacgagtaccagccagagatgctcgagaagttccggaacatgcgcagccagcacccctacgtcctcacggaggacaccctgaaggtctacctgaacaagttcagacagtcctcgccggacagcgtgaaggtggtcattcattttactgatggagcagacggagatctggctgatttacacagagcatctgagaacctccgccaagaaggagtccgtgccttgatcctggtgggccttgaacgagtggtcaacttggagcggctaatgcatctggagtttgggcgagggtttatgtatgacaggcccctgaggcttaacttgctggacttggattatgaactagcggagcagcttgacaacattgccgagaaagcttgctgtggggttccctgcaagtgctctgggcagaggggagaccgcgggcccatcggcagcatcgggccaaagggtattcctggagaagacggctaccgaggctatcctggtgatgagggtggacccggtgagcgtggtccgcctggtgtgaacggcactcaaggtttccagggctgcccgggccagagaggagtaaagggctctcggggattcccaggagagaagggcgaagtaggagaaattggactggatggtctggatggtgaagatggagacaaaggattgcctggttcttctggagagaaagggaatcctggaagaaggggtgataaaggacctcgaggagagaaaggagaaagaggagatgttgggattcgaggggacccgggtaacccaggacaagacagccaggagagaggacccaaaggagaaaccggtgacctcggccccatgggtgtcccagggagagatggagtacctggaggacctggagaaactgggaagaatggtggctttggccgaaggggaccccccggagctaagggcaacaagggcggtcctggccagccgggctttgagggagagcaggggaccagaggtgcacagggcccagctggtcctgctggtcctccagggctgataggagaacaaggcatttctggacctcggggaagcggaggtgccgctggtgctcctggagaacgaggcagaaccggtccactgggaagaaagggtgagcccggagagccaggaccaaaaggaggaatcgggaaccggggccctcgtggggagacgggagatgacgggagagacggagttggcagtgaaggacgcagaggcaaaaaaggagaaagaggattccctggatacccaggaccaaagggtaacccaggtgaacctgggctaaatggaacaacaggacccaaaggcatcagaggccgaaggggaaattcgggacctccagggatagttggacagaagggagaccctggctacccaggaccagctggtcccaagggcaacaggggcgactccatcgatcaatgtgccctcatccaaagcatcaaagataaatgcccttgctgttacgggcccctggagtgccccgtcttcccaacagaactagcctttgctttagacacctctgagggagtcaaccaagacactttcggccggatgcgagatgtggtcttgagtattgtgaatgacctgaccattgctgagagcaactgcccacggggggcccgggtggctgtggtcacctacaacaacgaggtgaccacggagatccggtttgctgactccaagaggaagtcggtcctcctggacaagattaagaaccttcaggtggctctgacatccaaacagcagagtctggagactgccatgtcgtttgtggccaggaacacatttaagcgtgtgaggaacggattcctaatgaggaaagtggctgttttcttcagcaacacacccacaagagcatccccacagctcagagaggctgtgctcaagctctcagatgcggggatcacccccttgttccttacaaggcaggaagaccggcagctcatcaacgctttgcagatcaataacacagcagtggggcatgcgcttgtcctgcctgcagggagagacctcacagacttcctggagaatgtcctcacgtgtcatgtttgcttggacatctgcaacatcgacccatcctgtggatttggcagttggaggccttccttcagggacaggagagcggcagggagcgatgtggacatcgacatggctttcatcttagacagcgctgagaccaccaccctgttccagttcaatgagatgaagaagtacatagcgtacctggtcagacaactggacatgagcccagatcccaaggcctcccagcacttcgccagagtggcagttgtgcagcacgcgccctctgagtccgtggacaatgccagcatgccacctgtgaaggtggaattctccctgactgactatggctccaaggagaagctggtggacttcctcagcaggggaatgacacagttgcagggaaccagggccttaggcagtgccattgaatacaccatagagaatgtctttgaaagtgccccaaacccacgggacctgaaaattgtggtcctgatgctgacgggcgaggtgccggagcagcagctggaggaggcccagagagtcatcctgcaggccaaatgcaagggctacttcttcgtggtcctgggcattggcaggaaggtgaacatcaaggaggtatacaccttcgccagtgagccaaacgacgtcttcttcaaattagtggacaagtccaccgagctcaacgaggagcctttgatgcgcttcgggaggctgttgccatccttcgtcagcagtgaaaatgctttttacttgtccccagatatcaggaaacagtgtgattggttccaaggggaccaacccacaaagaaccttgtgaagtttggtcacaaacaagtaaatgttccgaataacgttacttcaagtcctacatccaacccagtgacgacaacgaagccggtgactacgacgaagccggtgaccaccacaacaaagcctgtaaccaccacaacaaagcctgtgactattataaatcagccatctgtgaagccagccgctgcaaagccggcccctgcgaaacctgtggctgccaagcctgtggccacaaagatggccactgttagacccccagtggcggtgaagccagcaacggcagcgaagcctgtagcagcaaagccagcagctgtaagaccccccgctgctgctgctgcaaaaccagtggcgaccaagcctgaggtccctaggccacaggcagccaaaccagctgccaccaagccagccaccactaagcccatggttaagatgtcccgtgaagtccaggtgtttgagataacagagaacagcgccaaactccactgggagagggctgagccccccggtccttatttttatgacctcaccgtcacctcagcccatgatcagtccctggttctgaagcagaacctcacggtcacggaccgcgtcattggaggcctgctcgctgggcagacataccatgtggctgtggtctgctacctgaggtctcaggtcagagccacctaccacggaagtttcagtacaaagaaatctcagcccccacctccacagccagcaaggtcagcttctagttcaaccatcaatctaatggtgagcacagaaccattggctctcactgaaacagatatatgcaagttgccgaaagacgaaggaacttgcagggatttcatattaaaatggtactatgatccaaacaccaaaagctgtgcaagattctggtatggaggttgtggtggaaacgaaaacaaatttggatcacagaaagaatgtgaaaaggtttgcgctcctgtgctcgccaaacccggagtcatcagtgtgatgggaacctaagcgtgggtggccaacatcatatacctcttgaagaagaaggagtcagccatcgccaacttgtctctgtagaagctccgggtgtagattcccttgcactgtatcatttcatgctttgatttacactcgaactcgggagggaacatcctgctgcatgacctatcagtatggtgctaatgtgtctgtggaccctcgctctctgtctccaggcagttctctcgaatactttgaatgttgtgtaacagttagccactgctggtgtttatgtgaacattcctatcaatccaaattccctctggagtttcatgttatgcctgttgcaggcaaatgtaaagtctagaaaataatgcaaatgtcacggctactctatatacttttgcttggttcattttttttcccttttagttaagcatgactttagatgggaagcctgtgtatcgtggagaaacaagagaccaactttttcattccctgcccccaatttcccagactagatttcaagctaattttctttttctgaagcctctaacaaatgatctagttcagaaggaagcaaaatcccttaatctatgtgcaccgttgggaccaatgccttaattaaagaatttaaaaaagttgtaatagagaatatttttggcattcctctaatgttgtgtgttttttttttgtgtgtgctggagggaggggatttaattttaattttaaaatgtttaggaaatttatacaaagaaactttttaataaagtatattgaaagtttcctgggaaaaaaaaaaaaaaaaaa

SEQ ID NO: 9 - DNA, 4771 bp, Homo sapiens
ctctgtttgtacacagtgcgctcccggcggcccgctcgctcccctccagctcacgcttcattgttctccaagtcagaagccccgcagccgccgcgcggagaacagcgacagccgagcgcccggtccgcctgtctgccggtgggtctgcctgcccgcgcagcagacccggggcggccgcgggagcccgcgccccgcccgccgcgcctctgccgggacccacccgcagcggagggctgagcccgccggcggctccccggagctcacccacctccgcgcgccggagcgcaggcaaaaggggaggaaaggctcctctctttagtcaccactctcgccctctccaagaatttgtttaacaaagcgctgaggaaagagaacgtcttcttgaattctttagtaggggcggagtctgctgctgccctgcgctgccacctcggctacactgccctccgcgacgacccctgaccagccggggtcacgtccgggagacgggatcatgaagcgctcggtagccgtctggctcttggtcgggctcagcctcggtgtcccccagttcggcaaaggtgatatttgtgatcccaatccatgtgaaaatggaggtatctgtttgccaggattggctgatggttccttttcctgtgagtgtccagatggcttcacagaccccaactgttctagtgttgtggaggttgcatcagatgaagaagaaccaacttcagcaggtccctgcactcctaatccatgccataatggaggaacctgtgaaataagtgaagcataccgaggggatacattcataggctatgtttgtaaatgtccccgaggatttaatgggattcactgtcagcacaacataaatgaatgcgaagttgagccttgcaaaaatggtggaatatgtacagatcttgttgctaactattcctgtgagtgcccaggcgaatttatgggaagaaattgtcaatacaaatgctcaggcccactgggaattgaaggtggaattatatcaaaccagcaaatcacagcttcctctactcaccgagctctttttggactccaaaaatggtatccctactatgcacgtcttaataagaaggggcttataaatgcgtggacagctgcagaaaatgacagatggccgtggattcagataaatttgcaaaggaaaatgagagttactggtgtgattacccaaggagccaagaggattggaagcccagagtatataaaatcctacaaaattgcctacagtaatgatggaaagacttgggcaatgtacaaagtgaaaggcaccaatgaagacatggtgtttcgtggaaacattgataacaacactccatatgctaactctttcacaccccccataaaagctcagtatgtaagactctatccccaagtttgtcgaagacattgcactttgcgaatggaacttcttggctgtgaactgtcgggttgttctgagcctctgggtatgaaatcaggacatatacaagactatcagatcactgcctccagcatcttcagaacgctcaacatggacatgttcacttgggaaccaaggaaagctcggctggacaagcaaggcaaagtgaatgcctggacctctggccacaatgaccagtcacaatggttacaggtggatcttcttgttccaaccaaagtgactggcatcattacacaaggagctaaagattttggtcatgtacagtttgttggctcctacaaactggcttacagcaatgatggagaacactggactgtataccaggatgaaaagcaaagaaaagataaggttttccagggaaattttgacaatgacactcacagaaaaaatgtcatcgaccctcccatctatgcacgacacataagaatccttccttggtcctggtacgggaggatcacattgcggtcagagctgctgggctgcacagaggaggaatgaggggaggctacatttcacaaccctcttccctatttccctaaaagtatctccatggaatgaactgtgcaaaatctgtaggaaactgaatggtttttttttttttttcatgaaaaagtgctcaaattatggtaggcaactaacggtgtttttaagggggtctaagcctgccttttcaatgatttaatttgattttattttatccgtcaaatctcttaagtaacaacacattaagtgtgaattacttttctctcattgtttcctgaattattcgcattggtagaaatatattagggaaagaaagtagccttctttttatagcaagagtaaaaaagtctcaaagtcatcaaataagagcaagagttgatagagcttttacaatcaatactcacctaattctgataaaaggaatactgcaatgttagcaataagtttttttcttctgtaatgactctacgttatcctgtttccctgtgcctaccaaacactgtcaatgtttattacaaaattttaaagaagaatatgtaacatgcagtactgatattataattctcattttactttcattatttctaataagagattatgtgacttctttttcttttagttctattctacattcttaatattgtatattacctgaataattcaatttttttctaattgaatttcctattagttgactaaaagaagtgtcatgtttactcatatatgtagaacatgactgcctatcagtagattgatctgtatttaatattcgttaattaaatctgcagttttatttttgaaggaagccataactatttaatttccaaataattgcttcataaagaatcccatactctcagtttgcacaaaagaacaaaaaatatatatgtctctttaaatttaaatcttcatttagatggtaattacatatccttatatttactttaaaaaatcggcttatttgtttattttataaaaaatttagcaaagaaatattaatatagtgctgcatagtttggccaagcatactcatcatttctttgttcagctccacatttcctgtgaaactaacatcttattgagatttgaaactggtggtagtttcccaggaaggcacaggtggagttatttgtgagaagcaaagtgtttactaatgacaaagtagtaaaccattttcaagatgaaaactgatttctatttattttgcttcaaaggtcctgaaaaaataagcaattatcataacaatttgttattgatactggaggtttcattgacatgtctctcaaattaaagctcacactgcctccataaaagtcttcaacatctaatttataagctttacaagtatttattttataaggcttagacagaattattggagttttaaattaagtgtattggaaaagaaaggatggtatgtgtatgaaatgttaagatcctacgcaacactgctatttttttcctttaatatttgtgctgcataacaaaagccactagactgttactgtcttgtctgtccatgtgttaacagcatttcttaatgatgtatatatggagtggtcttcaatcatagtgaagaatttaaagagaaagtcaattgtattggcatttttaataagaacaaaattagttcgtctaaggggactggctggccacatatttgttccttgcccatatgctttctacttcttgttcttattatgaaattatgaatttgaagcctctgaaatggtgatcagttttcaacatctttcaaaaacaaaattactatttcctccatattgccttttttagataactttaaagttaggattttaaaatatttgtaactggctaaattttaaagtcgtgacaaataattacttaggttcagaaatatacacacacttactctttagccagtttctttcaaggtttactgtcccatcagatatctagccattttcctttgcaaattacataccttcttaagagtgtatttttaagattattacttacgctttatgatgatatagtttttcaaaattatttatagcttcatatgatgttttgtaattttttctattgatacctgttttaaaaatattttccaaggaagttgattaaaattatatttgttaccttttagaaaaagcattgaaatgagtttctcttgctttttcattttccctctgctttatatgctcttcgcaatacatcatgtccaacgggatacctattgttctcatgacacccaaaattgatgagagcaaaggggtcgcaccatatggaaatgttgaaaactattgtaaagtagtattatgaagtagcttttgtgtcattcatgtcgatgacatgaaagtgaagtaaatttattctatgtaaattcacactaaaaccagtacagtaccataagtagaatacatgtaagaatcacctagtcttcactatattgagtaaatataacatgctaattttacaattaatgaaactaaacttttaaacatctccattatatctacatccttttgaaggtatttatcatagttgccaattttaattttaggattgactttctctttctgaatgacttcataaagtttggtgtgaattttgaagacttgggttactaatgattgtatctttgctagtcaacaacttatgaaatatactcaatgcgtctgatgtgtcattaagtgcagaaataactaagacacaaataacctttgcaaaccttcaagctgtgtaatattccaatgttgtttttttctttgtatatatacttatatcacgtaggatgtaaaaccagtatgaccttgtctagtctccaaacttaaaataaacttttgaaaagctgggaaaaaaaaaaaa

SEQ ID NO: 10 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 11 - DNA, 3901 bp, Homo sapiens
gccacaggcgcggcgtcctcggcggcgggcggcagctagcgggagccgggacgccggtgcagccgcagcgcgcggaggaacccgggtgtgccgggagctgggcggccacgtccggtcgggaccgagacccctcgtagcgcattgcggcgacctcgccttccccggccgcgagcgcgccgctgcttgaaaagccgcggaacccaaggacttttctccggtccgagctcggggcgccccgcaggcgcacggtacccgtgctgcagctgggcacgccgcggcgccggggcctccgcaggcgccggcctgcgttctggaggaggggggcacaaggtctggagaccccgggtggcggacgggagccctccccccgccccgcctccgcgaccagctccgctccattgttcccgcccggctggaggcgccgagcaccgagcgcgccgggagtcgagcgccggccgcgagctcttgcgaccccgccagacccgaacagagcccgggggccggcgcggagccgggacgcgggcacacggcctcgcacaagccacgggcactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctccggaagtgacaccacctacttctccgtcaatgtttcagatgctctcccctcctcggaggatgatgatgatgatgatgactcctcttcagaggagaaagaaacagataacaccaaaccaaaccccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccgccccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgcctgggcccaccacctgtccgtccctgtcccctttcctgctggcaggagccggctgcctacaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctgagggcagggagtgggagccaatgaacaggcatgcaagtgagagcttcctgagctttctcctgtcggtttggtctgttttgccttcacccataagcccctcgcactctggtggcaggtgcttgtcctcagggctacagcagtagggaggtcagtgcttcgagccacgattgaaggtgacctctgccccagataggtggtgccagtggcttattaattccgatactagtttgctttgctgaccaaatgcctggtaccagaggatggtgaggcgaaggcaggttgggggcagtgttgtggcctggggccagccaacactggggctctgtatatagctatgaagaaaacacaaagttgataaatctgagtatatatttacatgtctttttaaaagggtcgttaccagagatttacccatcggtaagatgctcctggtggctgggaggcatcagttgctatatattaaaaacaaaaaaaaaa

SEQ ID NO: 12 - DNA, 2384 bp, Homo sapiens
atcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggtgttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggtgaggaaatccaaattggtcacattcccagggaagatgtagactatcacctgtacccacacggtccggggctcaatccaaatgcctctacaggacaagaagctctctctcagacaaccatctcatgggccccattccaggacacttctgagtacatcatttcatgtcatcctgttggcactgatgaagaacccttacagttcagggttcctggaacttctaccagtgcgactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttacgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaa

SEQ ID NO: 13 - DNA, 2042 bp, Homo sapiens
agtccgcctctggccagcttgggcggagcgcacggccagtgggaggtgctgagccgcctgatttattccggtcccagaggagaaggcgccagaaccccgcggggtctgagcagcccagcgtgcccattccagcgcccgcgtccccgcagcatgccgcgcccccgcctgctggccgcgctgtgcggcgcgctgctctgcgcccccagcctcctcgtcgccctggatatctgttccaaaaacccctgccacaacggtggtttatgcgaggagatttcccaagaagtgcgaggagatgtcttcccctcgtacacctgcacgtgccttaagggctacgcgggcaaccactgtgagacgaaatgtgtcgagccactgggcctggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccacctgccacccccaggtcttcctgctttccatgggcccgctgcctcttggcttctcagcccctttaaatcaccatagggctggggactggggaaggggagggtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagggtgggcgtttggggccctctatgctggcttttcaccccagaggacacaggcagcttccaaaatatatttatcttcttcacgggaaaaaaaaaaaaaaaaaa

SEQ ID NO: 14 - DNA, 2277 bp, Homo sapiens
aatcgaaagtagactcttttctgaagcatttcctgggatcagcctgaccacgctccatactgggagaggcttctgggtcaaaggaccagtctgcagagggatcctgtggctggaagcgaggaggctccacacggccgttgcagctaccgcagccaggatctgggcatccaggcacggccatgacccctccgaggctcttctgggtgtggctgctggttgcaggaacccaaggcgtgaacgatggtgacatgcggctggccgatgggggcgccaccaaccagggccgcgtggagatcttctacagaggccagtggggcactgtgtgtgacaacctgtgggacctgactgatgccagcgtcgtctgccgggccctgggcttcgagaacgccacccaggctctgggcagagctgccttcgggcaaggatcaggccccatcatgctggatgaggtccagtgcacgggaaccgaggcctcactggccgactgcaagtccctgggctggctgaagagcaactgcaggcacgagagagacgctggtgtggtctgcaccaatgaaaccaggagcacccacaccctggacctctccagggagctctcggaggcccttggccagatctttgacagccagcggggctgcgacctgtccatcagcgtgaatgtgcagggcgaggacgccctgggcttctgtggccacacggtcatcctgactgccaacctggaggcccaggccctgtggaaggagccgggcagcaatgtcaccatgagtgtggatgctgagtgtgtgcccatggtcagggaccttctcaggtacttctactcccgaaggattgacatcaccctgtcgtcagtcaagtgcttccacaagctggcctctgcctatggggccaggcagctgcagggctactgcgcaagcctctttgccatcctcctcccccaggacccctcgttccagatgcccctggacctgtatgcctatgcagtggccacaggggacgccctgctggagaagctctgcctacagttcctggcctggaacttcgaggccttgacgcaggccgaggcctggcccagtgtccccacagacctgctccaactgctgctgcccaggagcgacctggcggtgcccagcgagctggccctactgaaggccgtggacacctggagctggggggagcgtgcctcccatgaggaggtggagggcttggtggagaagatccgcttccccatgatgctccctgaggagctctttgagctgcagttcaacctgtccctgtactggagccacgaggccctgttccagaagaagactctgcaggccctggaattccacactgtgcccttccagttgctggcccggtacaaaggcctgaacctcaccgaggatacctacaagccccggatttacacctcgcccacctggagtgcctttgtgacagacagttcctggagtgcacggaagtcacaactggtctatcagtccagacgggggcctttggtcaaatattcttctgattacttccaagccccctctgactacagatactacccctaccagtccttccagactccacaacaccccagcttcctcttccaggacaagagggtgtcctggtccctggtctacctccccaccatccagagctgctggaactacggcttctcctgctcctcggacgagctccctgtcctgggcctcaccaagtctggcggctcagatcgcaccattgcctacgaaaacaaagccctgatgctctgcgaagggctcttcgtggcagacgtcaccgatttcgagggctggaaggctgcgattcccagtgccctggacaccaacagctcgaagagcacctcctccttcccctgcccggcagggcacttcaacggcttccgcacggtcatccgccccttctacctgaccaactcctcaggtgtggactagacggcgtggcccaagggtggtgagaaccggagaaccccaggacgccctcactgcaggctcccctcctcggcttccttcctctctgcaatgaccttcaacaaccggccaccagatgtcgccctactcacctgagcgctcagcttcaagaaattactggaaggcttccactagggtccaccaggagttctcccaccacctcaccagtttccaggtggtaagcaccaggacgccctcgaggttgctctgggatccccccacagcccctggtcagtctgcccttgtcactggtctgaggtcattaaaattacattgaggttcctacaaaaaaaaaaaaaaaaaa

SEQ ID NO: 15 - DNA, 2298 bp, Homo sapiens
tgtgctcgctgctcagcgcgcacccggaagatgaggctcgccgtgggagccctgctggtctgcgccgtcctggggctgtgtctggctgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaaaatctcagaggtagggctgccaccaaggtgaagatgggaacgcagatgatccatgagtttgccctggtttcactggcccaagtggtttgtgctaaccacgtctgtcttcacagctctgtgttgccatgtgtgctgaacaaaaaataaaaattattattgattttatatttc

SEQ ID NO: 16 - DNA, 4017 bp, Homo sapiens
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctctatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctctttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcctgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctag

SEQ ID NO: 17 - DNA, 101 bp, Homo sapiens
ccgcccccgcgtctccagggcaaccgtggctttcgattgttactgtgggaactggaggtaacagtctacagccatggtcgccccgcagcacgcccacgcgc

SEQ ID NO: 18 - DNA, 9309 bp, Artificial Sequence
  Description of Artificial Sequence: Synthetic polynucleotide
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattcgagctcggtacccccgcccccgcgtctccagggcaaccgtggctttcgattgttactgtgggaactggaggtaacagtctacagccatggtcgccccgcagcacgcccacgcgcgatatcgggcccgcggtaccgtcgactgcagaattctaccgggtaggggaggcgcttttcccaaggcagtctggagcatgcgctttagcagccccgctggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctcgcacgcttcaaaagcgcacgtctgccgcgctgttctcctcttcctcatctccgggcctttcgaccatctagatccaccggtcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaggcggccggggtcgactgatccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggatcaaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 19 - DNA, 627 bp, Homo sapiens
gaattcgcccttcctgagatcaccggtaggagggccatcatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgaaagggcgaattc